(19) United States
(12) Patent Application Publication
(10) Pub. No.: US 2021/0188927 A1
(43) Pub. Date: Jun. 24, 2021

(54) COMPOSITIONS AND METHODS FOR TREATING AGE-RELATED MACULAR DEGENERATION

(71) Applicant: Gemini Therapeutics, Inc., Cambridge, MA (US)

(72) Inventors: James McLaughlin, Cambridge, MA (US); Adarsha Koirala, Cambridge, MA (US)

(21) Appl. No.: 16/757,268

(22) PCT Filed: Oct. 19, 2018
(86) PCT No.: PCT/US2018/056709

§ 371 Date: Apr. 17, 2020

Related U.S. Application Data
(60) Provisional application No. 62/574,814, filed on Oct. 20, 2017.

(51) Int. Cl. C07K 14/47; C12N 15/86

(52) U.S. Cl. CPC C07K 14/472; C12N 15/86; C12N 2840/007; C12N 2800/60; C12N 2750/14141

(57) ABSTRACT

The present disclosure provides compositions and methods for treating, preventing, or inhibiting diseases of the eye. In one aspect, the disclosure provides recombinant CFH FHL-1 adeno-associated virus (rAAV) vectors comprising a complement system gene.

### CROSS-REFERENCE TO RELATED APPLICATION

This application claims the benefit of priority to U.S. Provisional Application Ser. No. 62/574,814, filed Oct. 20, 2017. The disclosure of the foregoing application is hereby incorporated by reference in its entirety.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits. The size and number of drusen deposits typically correlates with AMD severity. AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision.

### SUMMARY OF THE DISCLOSURE

In some embodiments, the disclosure provides for an adeno-associated viral (AAV) vector encoding a Complement Factor H (CFH) or human Factor H Like 1 (FHL1) protein or biologically active fragment thereof, wherein the vector comprises a nucleotide sequence that is at least 80% identical to the nucleotide sequence of SEQ ID NO: 1-3 or 5, or a fragment thereof. In some embodiments, the nucleotide sequence is at least 90% identical to the nucleotide sequence of SEQ ID NO: 1-3 or 5, or codon-optimized variant and/or a fragment thereof. In some embodiments, the nucleotide sequence is at least 95% identical to the nucleotide sequence of SEQ ID NO: 1-3 or 5, or codon-optimized variant and/or a fragment thereof. In some embodiments, the nucleotide sequence is the sequence of SEQ ID NO: 1-3 or 5, or codon-optimized variant and/or a fragment thereof. In some embodiments, the vector encodes a CFH protein or biologically active fragment thereof comprising at least four CCP domains. In some embodiments, the vector encodes a CFH protein or biologically active fragment thereof comprising at least five CCP domains. In some embodiments, the vector encodes a CFH protein or biologically active fragment thereof comprising at least six CCP domains. In some embodiments, the vector encodes a CFH protein or biologically active fragment thereof comprising at least seven CCP domains. In some embodiments, the vector encodes a CFH protein or biologically active fragment thereof comprising at least three CCP domains. In some embodiments, the vector encodes a CFH protein or biologically active fragment thereof comprising the H402 polymorphism. In some embodiments, the vector encodes a CFH protein or biologically active fragment thereof comprising the V62 polymorphism. In some embodiments, the CFH protein or biologically active fragment thereof comprises the amino acid sequence of SEQ ID NO: 4. In some embodiments, the amino acid sequence of SEQ ID NO: 4 is the C-terminal sequence of the CFH protein. In some embodiments, the CFH protein or biologically active fragment thereof is capable of diffusing across the Bruch's membrane. In some embodiments, the CFH protein or biologically active fragment thereof is capable of binding C3b. In some embodiments, the CFH protein or biologically active fragment thereof is capable of facilitating the breakdown of C3b. In some embodiments, the vector comprises a promoter that is less than 1000 nucleotides in length. In some embodiments, the vector comprises a promoter that is less than 500 nucleotides in length. In some embodiments, the vector comprises a promoter that is less than 400 nucleotides in length. In some embodiments, the promoter comprises a promoter having the nucleotide sequence of SEQ ID NO: 6, or a fragment thereof. In some embodiments, the promoter comprises a promoter having the nucleotide sequence of SEQ ID NO: 8, or a fragment thereof. In some embodiments, the promoter comprises a promoter having the nucleotide sequence of SEQ ID NO: 12, or a fragment thereof. In some embodiments, the promoter comprises a promoter having the nucleotide sequence of SEQ ID NO: 14, or a fragment thereof. In some embodiments, the promoter comprises a promoter having the nucleotide sequence of SEQ ID NO: 16, or a fragment thereof. In some embodiments, the promoter comprises a promoter having the nucleotide sequence of SEQ ID NO: 18, or a fragment thereof. In some embodiments, the promoter comprises a promoter having the nucleotide sequence of SEQ ID NO: 20, or a fragment thereof. In some embodiments, the promoter comprises a promoter having the nucleotide sequence of SEQ ID NO: 31, or a fragment thereof. In some embodiments, the promoter comprises a promoter having the nucleotide sequence of SEQ ID NO: 32, or a fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 6, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 8, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 12, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 14, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 16, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 18, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 20, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 31, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 32, or a biologically active fragment thereof. In some embodiments, the promoter comprises a promoter having a nucleotide sequence that is at least 65%, 70%, 75%, 80%, 85%, 90%, 92%, 93%, 95%, 98%, or 99% identical to the nucleotide sequence of SEQ ID NO: 32, or a biologically active fragment thereof. In some embodiments, the promoter comprises an additional viral intron. In some embodiments, the additional viral intron comprises the nucleotide sequence of SEQ ID NO: 10, or a fragment thereof. In some embodiments, the vector is an AAV2 vector. In some embodiments, the vector comprises a CMV promoter. In some embodiments, the vector comprises a Kozak sequence. In some embodiments, the vector comprises one or more ITR sequence flanking the vector portion encoding CFH. In some embodiments, the vector comprises a polyadenylation sequence. In some embodiments, the vector comprises a selective marker. In some embodiments, the selective marker is an antibiotic-resistance gene. In some embodiments, the antibiotic-resistance gene is an ampicillin-resistance gene.

In some embodiments, the disclosure provides a composition comprising any of the vectors disclosed herein and a pharmaceutically acceptable carrier.

In some embodiments, the disclosure provides for a method of treating a subject having a disorder associated with undesired activity of the alternative complement pathway, comprising the step of administering to the subject any of the vectors disclosed herein or any of the compositions disclosed herein.

In some embodiments, the disclosure the provides for a method of treating a subject having age-related macular degeneration (AMD), comprising the step of administering to the subject any of the vectors disclosed herein or any of the compositions disclosed herein. In some embodiments, the the vector or composition is administered intravitreally. In some embodiments, the subject is not administered a protease or a polynucleotide encoding a protease. In some embodiments, the subject is not administered a furin protease or a polynucleotide encoding a furin protease. In some embodiments, the subject is a human. In some embodiments, the human is at least 40 years of age. In some embodiments, the human is at least 50 years of age. In some embodiments, the human is at least 65 years of age. In some embodiments, the vector or composition is administered locally. In some embodiments, the vector or composition is administered systemically. In some embodiments, the vector or composition comprises a promoter that is associated with strong expression in the liver. In some embodiments, the promoter comprises a nucleotide sequence that is at least 90%, 95% or 100% identical to the nucleotide sequence of any one of SEQ ID NOs: 16, 18, or 20. In some embodiments, the vector or composition comprises a promoter that is associated with strong expression in the eye. In some embodiments, the promoter comprises a nucleotide sequence that is at least 90%, 95%, or 100% identical to the nucleotide sequence of any one of SEQ ID NOs: 6 or 32. In some embodiments, the subject has a loss-of-function mutation in the subject's CFI gene. In some embodiments, the subject has one or more CFI mutations selected from the group consisting of: G119R, L131R, V152M, G162D, R187Y, R187T, T2031, A240G, A258T, G287R, A300T, R317W, R339Q, V412M, and P553S. In some embodiments, the subject has a loss-of-function mutation in the subject's CFH gene. In some embodiments, the subject has one or more CFH mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. In some embodiments, the subject has atypical hemolytic uremic syndrome (aHUS). In some embodiments, the subject is suffering from a renal disease or complication. In some embodiments, any of the vectors disclosed herein or any of the compositions disclosed herein is capable of inducing at least 20%, 50%, 100%, 150%, 200%, 250%, 300%, 400%, 500%, 700%, 900%, 1000%, 1100%, 1500%, or 2000% higher expression of CFH or FHL in a target cell (e.g., an RPE or liver cell) as compared to the endogenous expression of CFH or FHL in the target cell. In some embodiments, the expression of any of the vectors disclosed herein or any of the compositions disclosed herein (e.g., an RPE or liver cell) results in at least 20%, 50%, 100%, 150%, 200%, 250%, 300%, 400%, 500%, 700%, 900%, 1000%, 1100%, 1500%, or 2000% higher levels of CFH or FHL activity in the target cell as compared to endogenous levels of CFH or FHL activity in the target cell. In some embodiments, any of the vectors disclosed herein or any of the compositions disclosed herein induces CFH expression in a target cell of the eye. In some embodiments, any of the vectors or compositions disclosed herein induces CFH expression in a target cell of the retina or macula. In some embodiments, target cell of the retina is selected from the group of layers consisting of: inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE). In some embodiments, the target cell is in the choroid plexus. In some embodiments, the target cell is in the macula. In some embodiments, any of the vectors or compositions disclosed herein induces CFH expression in a cell of the GCL and/or RPE. In some embodiments, the vector or composition is administered to the retina at a dose in the range of 1×10¹⁰ vg/eye to 1×10¹³ vg/eye. In some embodiments, the vector or composition is administered to the retina at a dose of about 1.4×10¹² vg/eye. In some embodiments, the promoter comprises a promoter having the nucleotide sequence of SEQ ID NO: 36, or a fragment thereof.

### BRIEF DESCRIPTION OF THE DRAWINGS

FIG. 1 shows a vector map of a full vector genome construct for expression of CFH. “ITR” corresponds to inverted terminal repeats; “CRALBP promoter” corresponds to the cellular retinaldehyde-binding protein promoter; “CFH” corresponds to the gene encoding Complement Factor H; “polyA” corresponds to the polyadenylation sequence; “Amp R” corresponds to the ampicillin resistance cassette. The nucleotide sequence corresponding to the vector illustrated in FIG. 1 is SEQ ID NO: 7.

FIG. 2 shows a vector map of a full vector genome construct for expression of CFH. “ITR” corresponds to inverted terminal repeats; “EF1a promoter” corresponds to the elongation factor-1 alpha promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 2 is SEQ ID NO: 9.

FIG. 3 shows a vector map of a full vector genome construct for expression of CFH. “ITR” corresponds to inverted terminal repeats; “EF1a.SV40i” corresponds to the elongation factor-1 alpha promoter including the simian virus 40 intron; “CFH” corresponds to the gene encoding Complement Factor H; “polyA” corresponds to the polyadenylation sequence; “AmpicillinR” corresponds to the ampicillin resistance cassette. The nucleotide sequence corresponding to the vector illustrated in FIG. 3 is SEQ ID NO: 11.

FIG. 4 shows a vector map of a full vector genome construct for expression of CFH. “ITR” corresponds to inverted terminal repeats; “HSP70 promoter” corresponds to the heat shock protein 70 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 4 is SEQ ID NO: 13.

FIG. 5 shows a vector map of a full vector genome construct for expression of CFH. “ITR” corresponds to inverted terminal repeats; “sCBA promoter” corresponds to the chicken 13 actin promoter; “CFH” corresponds to the gene encoding Complement Factor H; “polyA” corresponds to the polyadenylation sequence; “AmpR” corresponds to the ampicillin resistance cassette. This vector also included the SV40i intron. The nucleotide sequence corresponding to the vector illustrated in FIG. 5 is SEQ ID NO: 15.

FIG. 6 shows a vector map of a full vector genome construct for expression of CFH. “ITR” corresponds to inverted terminal repeats; “AAT1” corresponds to the alpha-1 antitrypsin 1 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 6 is SEQ ID NO: 17.

FIG. 7 shows a vector map of a full vector genome construct for expression of CFH. “ITR” corresponds to inverted terminal repeats; “ALB” corresponds to a synthetic promoter based on the human albumin promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 7 is SEQ ID NO: 19.

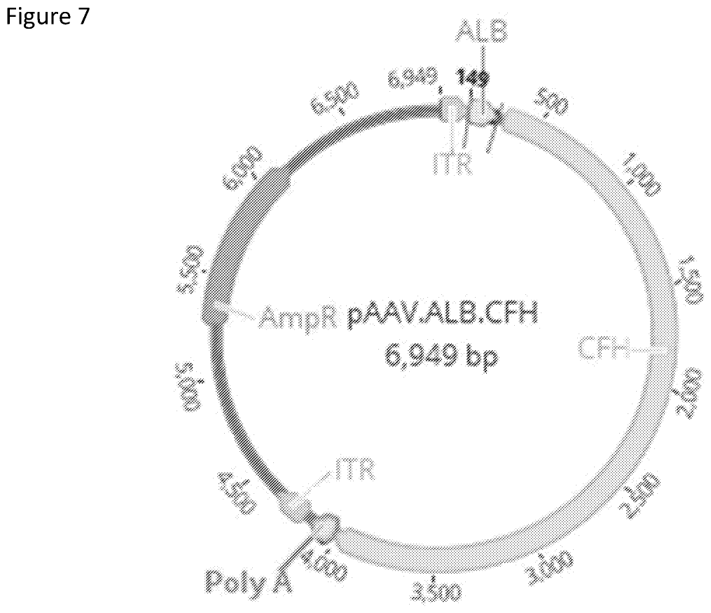

FIG. 8 shows a vector map of a full vector genome construct for expression of CFH. “ITR” corresponds to inverted terminal repeats; “PCK1” corresponds to the phosphoenolpyruvate carboxykinase 1 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 8 is SEQ ID NO: 21.

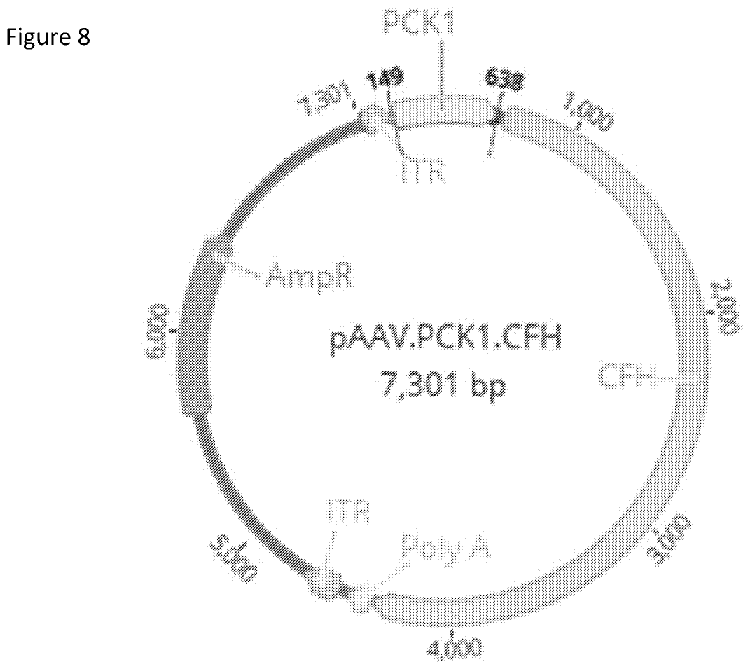

FIG. 9 shows a vector map of a full vector genome construct for expression of FHL-1. “ITR” corresponds to inverted terminal repeats; “EF1a” corresponds to the elongation factor-1 alpha promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 9 is SEQ ID NO: 22.

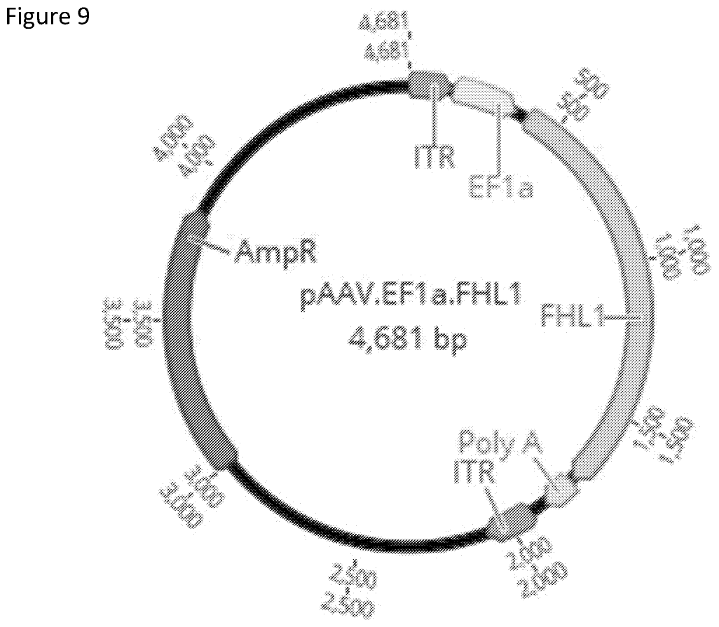

FIG. 10 shows a vector map of a full vector genome construct for expression of FHL-1. “ITR” corresponds to inverted terminal repeats; “ALB” corresponds to a synthetic promoter based on the human albumin promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 10 is SEQ ID NO: 23.

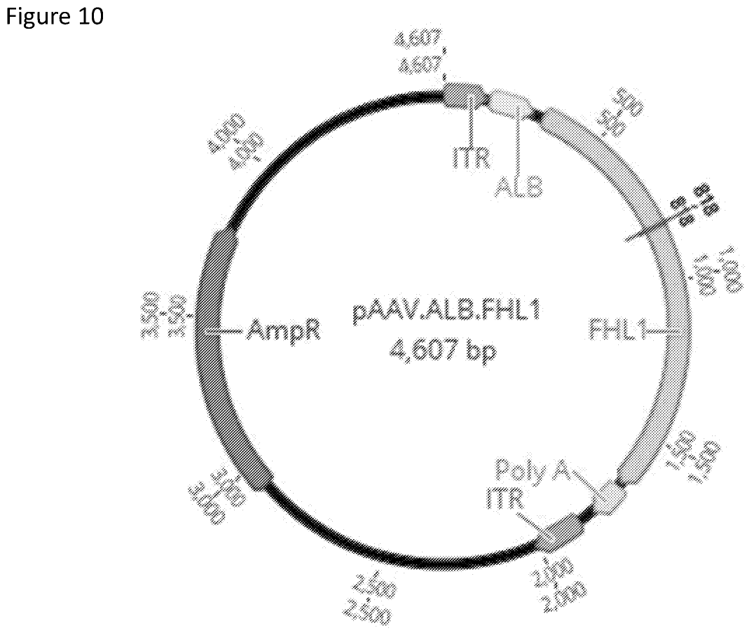

FIG. 11 shows a vector map of a full vector genome construct for expression of FHL-1. “ITR” corresponds to inverted terminal repeats; “AAT1” corresponds to the alpha-1 antitrypsin 1 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 11 is SEQ ID NO: 24.

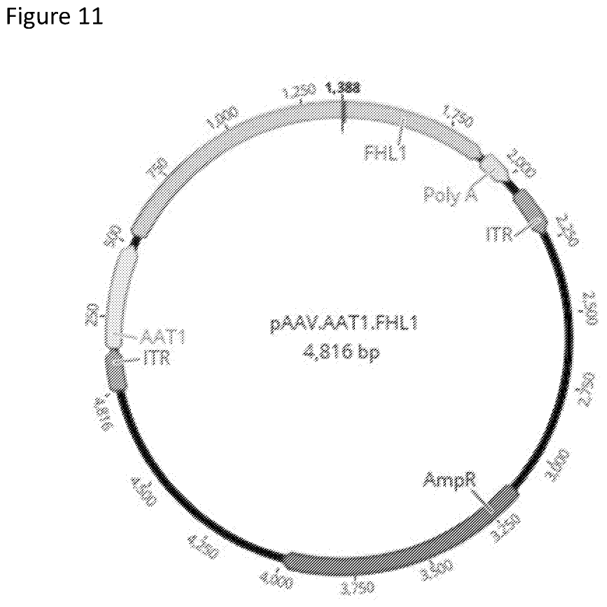

FIG. 12 shows a vector map of a full vector genome construct for expression of FHL-1. “ITR” corresponds to inverted terminal repeats; “EF1a.SV40i” corresponds to the elongation factor-1 alpha promoter including the simian virus 40 intron; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 12 is SEQ ID NO: 25.

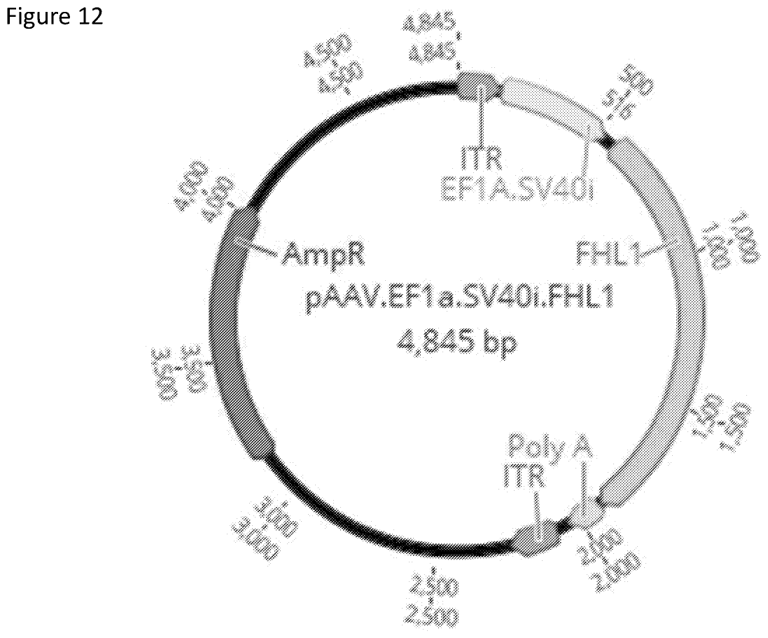

FIG. 13 shows a vector map of a full vector genome construct for expression of FHL-1. “ITR” corresponds to inverted terminal repeats; “CAG” corresponds to a synthetic promoter that includes the cytomegalovirus (CMV) early enhancer element, the promoter/first exon/first intron of chicken beta-actin gene, and the splice acceptor of the rabbit beta-globin gene; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 13 is SEQ ID NO: 26.

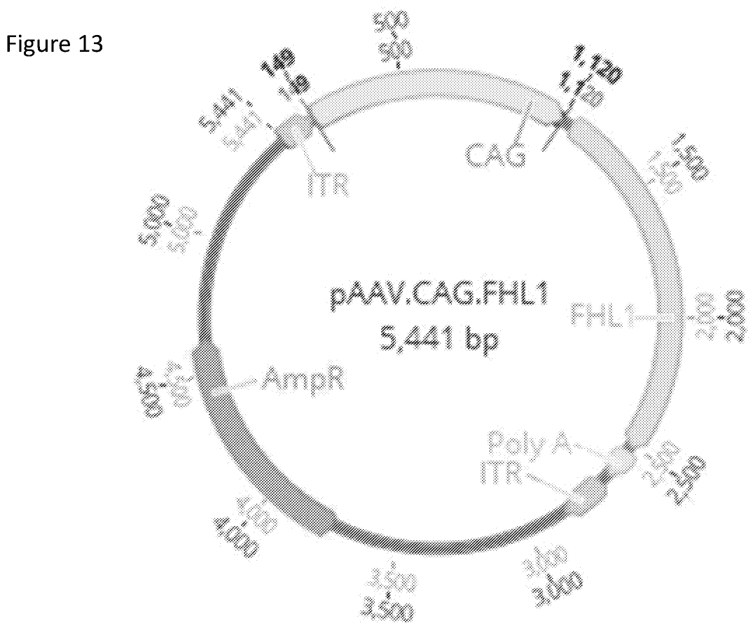

FIG. 14 shows a vector map of a full vector genome construct for expression of FHL-1. “ITR” corresponds to inverted terminal repeats; “CRALBP” corresponds to the cellular retinaldehyde-binding protein promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 14 is SEQ ID NO: 27.

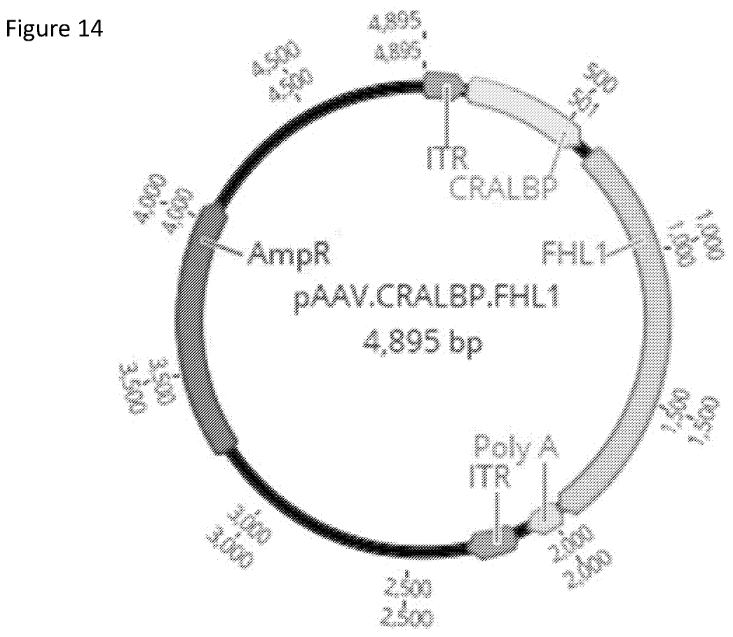

FIG. 15 shows a vector map of a full vector genome construct for expression of FHL-1. “ITR” corresponds to inverted terminal repeats; “hRPE65” corresponds to the retinal pigment epithelial 65 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 15 is SEQ ID NO: 28.

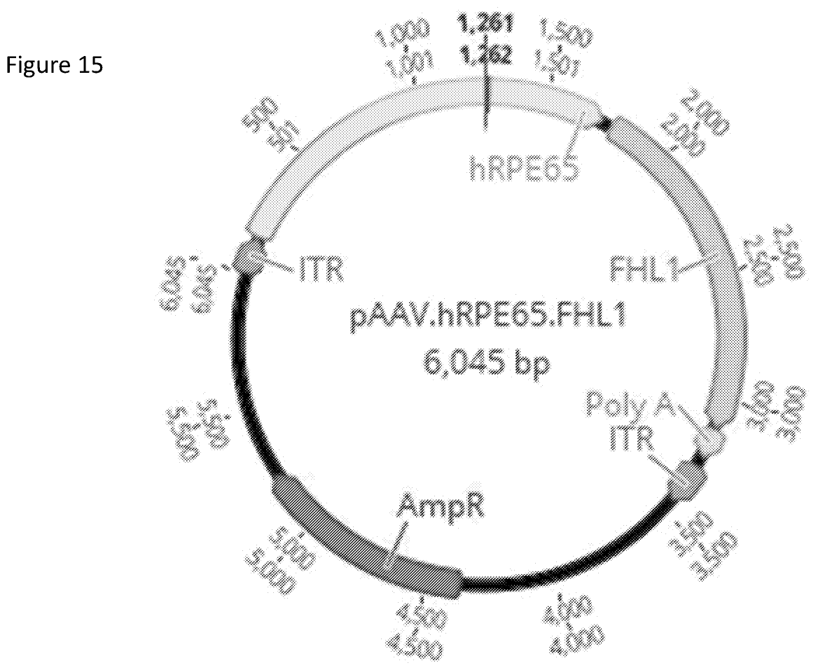

FIG. 16 shows a vector map of a full vector genome construct for expression of FHL-1. “ITR” corresponds to inverted terminal repeats; “HSP70” corresponds to the heat shock protein 70 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 16 is SEQ ID NO: 29.

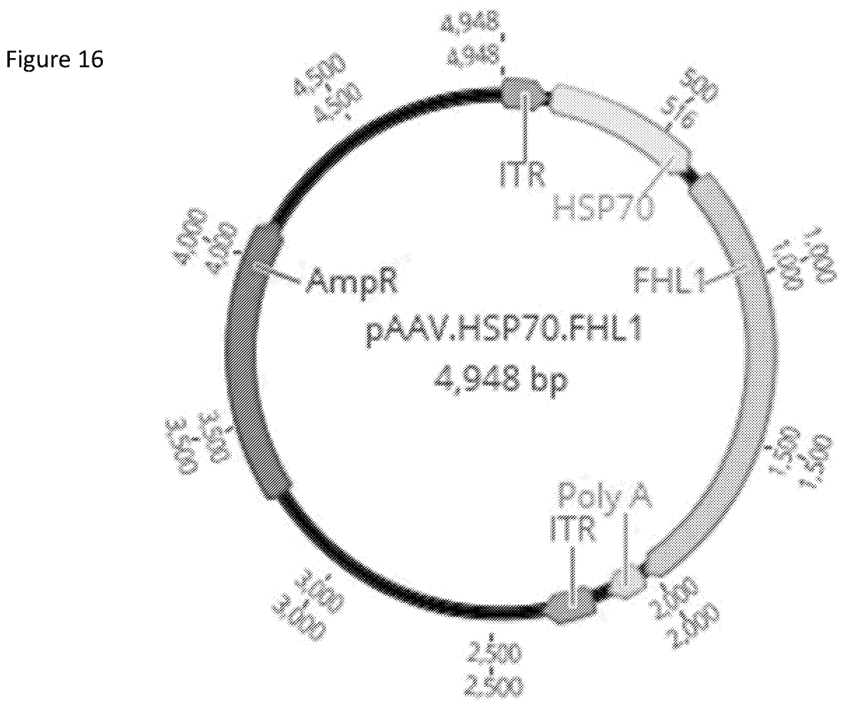

FIG. 17 shows a vector map of a full vector genome construct for expression of FHL-1. “ITR” corresponds to inverted terminal repeats; “PCK1” corresponds to the phosphoenolpyruvate carboxykinase 1 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 17 is SEQ ID NO: 30.

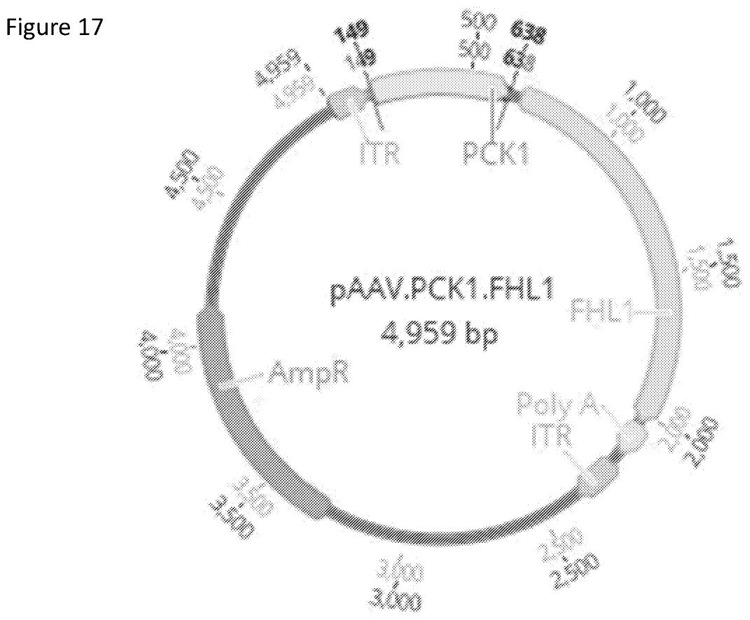

FIG. 18 shows a Western Blot from an experiment in which the levels of CFH (or the loading control GAPDH) were detected in HEK cells transfected with various CFH or control plasmids.

FIG. 19 shows a bar graph comparing the levels of CFH protein levels from the Western analysis of FIG. 18 relative to GAPDH protein levels.

FIG. 20 shows a Western Blot from an experiment in which the levels of CFH or GFP (or the loading control GAPDH) were detected in HEK cells transfected with various CFH or control AAV vectors.

### DETAILED DESCRIPTION OF THE DISCLOSURE

The disclosure provides compositions and methods for treating, preventing, or inhibiting diseases of the eye. In one aspect, the disclosure provides recombinant adeno-associated virus (rAAV) vectors comprising a complement system gene (such as, but not limited to genes encoding complement factor H (CFH) or factor-H-like protein 1 (FHL1)). In another aspect, the disclosure provides methods of treating, preventing, or inhibiting diseases of the eye by intraocularly (e.g., intravitreally) administering an effective amount of an rAAV vector of the disclosure to deliver and drive the expression of a complement factor gene.

A wide variety of diseases of the eye may be treated or prevented using the viral vectors and methods provided herein. Diseases of the eye that may be treated or prevented using the vectors and methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

### GENERAL TECHNIQUES

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the embodimented disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. The term host cell may refer to the packaging cell line in which the rAAV is produced from the plasmid. In the alternative, the term “host cell” may refer to the target cell in which expression of the transgene is desired.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector based on an adeno-associated virus comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. An rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR.

A “helper virus” for AAV refers to a virus that allows AAV (which is a defective parvovirus) to be replicated and packaged by a host cell. A number of such helper viruses are known in the art.

As used herein, “expression control sequence” means a nucleic acid sequence that directs transcription of a nucleic acid. An expression control sequence can be a promoter, such as a constitutive promoter, or an enhancer. The expression control sequence is operably linked to the nucleic acid sequence to be transcribed.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye.

In some embodiments, the subject has, or is at risk of developing a renal disease or complication. In some embodiments, the renal disease or complication is associated with AMD or aHUS.

In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

Each embodiment described herein may be used individually or in combination with any other embodiment described herein.

Construction of rAAV Vectors

The disclosure provides recombinant AAV (rAAV) vectors comprising a complement system gene (e.g. CFH, FHL1, FHR1, FHR2, FHR3, FHR4, or FHR5), a splice variant (e.g. FHL1), or a fragment thereof, under the control of a suitable promoter to direct the expression of the complement system gene, splice variant, or fragment thereof in the eye. The disclosure further provides a therapeutic composition comprising an rAAV vector comprising a complement system gene, a splice variant, or a fragment thereof (e.g. CFH, HILL FHR1, FHR2, FHR3, FHR4, or FHR5) under the control of a suitable promoter. A variety of rAAV vectors may be used to deliver the desired complement system gene to the eye and to direct its expression. More than 30 naturally occurring serotypes of AAV from humans and non-human primates are known. Many natural variants of the AAV capsid exist, and an rAAV vector of the disclosure may be designed based on an AAV with properties specifically suited for ocular cells. In certain embodiments, the complement system gene is a splice variant (e.g. FHL1, which is a truncated splice variant of CFH).

In general, an rAAV vector is comprised of, in order, a 5′ adeno-associated virus inverted terminal repeat, a transgene or gene of interest encoding a complement system polypeptide (e.g. CFH, FHL1, FHR1, FHR2, FHR3, FHR4, or FHR5) or a biologically active fragment thereof operably linked to a sequence which regulates its expression in a target cell, and a 3′ adeno-associated virus inverted terminal repeat. In addition, the rAAV vector may preferably have a polyadenylation sequence. Generally, rAAV vectors should have one copy of the AAV ITR at each end of the transgene or gene of interest, in order to allow replication, packaging, and efficient integration into cell chromosomes. Within preferred embodiments of the disclosure, the transgene sequence encoding a complement system polypeptide (e.g. CFH, FHL1, FHR1, FHR2, FHR3, FHR4, or FHR5) or a biologically active fragment thereof will be of about 2 to 5 kb in length (or alternatively, the transgene may additionally contain a “stuffer” or “filler” sequence to bring the total size of the nucleic acid sequence between the two ITRs to between 2 and 5 kb). Alternatively, the transgene encoding a complement system polypeptide (e.g. CFH, FHL1, FHR1, FHR2, FHR3, FHR4, or FHR5) or a biologically active fragment thereof may be composed of the same heterologous sequence several times (e.g., two nucleic acid molecules of a complement system gene separated by a ribosomal readthrough stop codon, or alternatively, by an Internal Ribosome Entry Site or “IRES”), or several different heterologous sequences (e.g., different complement system members such as FHL1, separated by a ribosomal readthrough stop codon or an IRES).

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV1 through AAV12. The tropism of the vector may be altered by packaging the recombinant genome of one serotype into capsids derived from another AAV serotype. In some embodiments, the ITRs of the rAAV virus may be based on the ITRs of any one of AAV1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In certain embodiments, the AAV capsid serotype is AAV2.

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein.

Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the transgene encoding the complement system polypeptide (e.g. CFH, FHL1, FHR1, FHR2, FHR3, FHR4, or FHR5) or biologically active fragment thereof to the nucleus. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). In some embodiments, vectors suitable for use with the present disclosure may be pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

An rAAV vector of the disclosure is generated by introducing a nucleic acid sequence encoding an AAV capsid protein, or fragment thereof, a functional rep gene or a fragment thereof; a minigene composed of, at a minimum, AAV inverted terminal repeats (ITRs) and a transgene encoding a complement system polypeptide (e.g. CFH, FHL1, FHR1, FHR2, FHR3, FHR4, or FHR5) or a biologically active fragment thereof; and sufficient helper functions to permit packaging of the minigene into the AAV capsid, into a host cell. The components required for packaging an AAV minigene into an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In some embodiments, such a stable host cell will contain the required component(s) under the control of an inducible promoter. Alternatively, the required component(s) may be under the control of a constitutive promoter. Examples of suitable inducible and constitutive promoters are provided herein, in the discussion below of regulator elements suitable for use with the transgene, i.e., a nucleic acid encoding a complement system polypeptide (e.g. CFH, FHL1, FHR1, FHR2, FHR3, FHR4, or FHR5) or biologically active fragment thereof. In still another alternative, a selected stable host cell may contain selected components under the control of a constitutive promoter and other selected components under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the disclosure may be delivered to the packaging host cell in the form of any genetic element which transfers the sequences. The selected genetic element may be delivered by any suitable method known in the art. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, 1993 J. Virol, 70:520-532 and U.S. Pat. No. 5,478,745, among others. These publications are incorporated by reference herein.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV10, AAV11, AAV12, AAV-DJ, AAV-DJ8, AAV-DJ9, AAVrh8, AAVrh8R or AAVrh10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene is composed of, at a minimum, a transgene encoding a complement system polypeptide (e.g. CFH, FHL1, FHR1, FHR2, FHR3, FHR4, or FHR5) or a biologically active fragment thereof, as described above, and its regulatory sequences, and 5′ and 3′ AAV inverted terminal repeats (ITRs). In one desirable embodiment, the ITRs of AAV serotype 2 are used. However, ITRs from other suitable serotypes may be selected. The minigene is packaged into a capsid protein and delivered to a selected host cell.

In some embodiments, regulatory sequences are operably linked to the transgene encoding a complement system polypeptide (e.g. CFH, FHL1, FHR1, FHR2, FHR3, FHR4, or FHR5) or a biologically active fragment thereof. The regulatory sequences may include conventional control elements which are operably linked to the complement system gene, splice variant, or a fragment thereof in a manner which permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Numerous expression control sequences, including promoters, are known in the art and may be utilized.

The regulatory sequences useful in the constructs of the present disclosure may also contain an intron, desirably located between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the method of the disclosure is an internal ribosome entry site (IRES). An IRES sequence, or other suitable systems, may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In some embodiments, expression of the transgene encoding a complement system polypeptide (e.g. CFH, FHL1, FHR1, FHR2, FHR3, FHR4, or FHR5) or a biologically active fragment thereof is driven by a separate promoter (e.g., a viral promoter). In certain embodiments, any promoters suitable for use in AAV vectors may be used with the vectors of the disclosure. The selection of the transgene promoter to be employed in the rAAV may be made from among a wide number of constitutive or inducible promoters that can express the selected transgene in the desired ocular cell. Examples of suitable promoters are described below.

Other regulatory sequences useful in the disclosure include enhancer sequences Enhancer sequences useful in the disclosure include the 1RBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are well-known and many such sequences are available. See, e.g., Sambrook et al, and references cited therein at, for example, pages 3.18-3.26 and 16, 17-16.27 and Ausubel et al., Current Protocols in Molecular Biology, John Wiley & Sons, New York, 1989).

The rAAV vector may also contain additional sequences, for example from an adenovirus, which assist in effecting a desired function for the vector. Such sequences include, for example, those which assist in packaging the rAAV vector in adenovirus-associated virus particles.

The rAAV vector may also contain a reporter sequence for co-expression, such as but not limited to lacZ, GFP, CFP, YFP, RFP, mCherry, tdTomato, etc. In some embodiments, the rAAV vector may comprise a selectable marker. In some embodiments, the selectable marker is an antibiotic-resistance gene. In some embodiments, the antibiotic-resistance gene is an ampicillin-resistance gene. In some embodiments, the ampicillin-resistance gene is beta-lactamase.

In some embodiments, the rAAV particle is an ssAAV. In some embodiments, the rAAV particle is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to −55 kd) and any currently available RNA-based therapy.

The single-stranded nature of the AAV genome may impact the expression of rAAV vectors more than any other biological feature. Rather than rely on potentially variable cellular mechanisms to provide a complementary-strand for rAAV vectors, it has now been found that this problem may be circumvented by packaging both strands as a single DNA molecule. In the studies described herein, an increased efficiency of transduction from duplexed vectors over conventional rAAV was observed in HeLa cells (5-140 fold). More importantly, unlike conventional single-stranded AAV vectors, inhibitors of DNA replication did not affect transduction from the duplexed vectors of the invention. In addition, the inventive duplexed parvovirus vectors displayed a more rapid onset and a higher level of transgene expression than did rAAV vectors in mouse hepatocytes in vivo. All of these biological attributes support the generation and characterization of a new class of parvovirus vectors (delivering duplex DNA) that significantly contribute to the ongoing development of parvovirus-based gene delivery systems.

Overall, a novel type of parvovirus vector that carries a duplexed genome, which results in co-packaging strands of plus and minus polarity tethered together in a single molecule, has been constructed and characterized by the investigations described herein. Accordingly, the present invention provides a parvovirus particle comprising a parvovirus capsid (e.g., an AAV capsid) and a vector genome encoding a heterologous nucleotide sequence, where the vector genome is self-complementary, i.e., the vector genome is a dimeric inverted repeat. The vector genome is preferably approximately the size of the wild-type parvovirus genome (e.g., the AAV genome) corresponding to the parvovirus capsid into which it will be packaged and comprises an appropriate packaging signal. The present invention further provides the vector genome described above and templates that encode the same.

rAAV vectors useful in the methods of the disclosure are further described in PCT publication No. WO2015168666 and PCT publication no. WO2014011210, the contents of which are incorporated by reference herein.

In some embodiments, any of the vectors disclosed herein is capable of inducing at least 20%, 50%, 100%, 150%, 200%, 250%, 300%, 400%, 500%, 700%, 900%, 1000%, 1100%, 1500%, or 2000% higher expression of CFH or FHL in a target cell (e.g., an RPE or liver cell) as compared to the endogenous expression of CFH or FHL in the target cell. In some embodiments, expression of any of the vectors disclosed herein in a target cell (e.g., an RPE or liver cell) results in at least 20%, 50%, 100%, 150%, 200%, 250%, 300%, 400%, 500%, 700%, 900%, 1000%, 1100%, 1500%, or 2000% higher levels of CFH or FHL activity in the target cell as compared to endogenous levels of CFH or FHL activity in the target cell.

Complement System Genes

In the search for causative factors associated with age related macular degeneration, epidemiological and genetic studies have identified numerous common and rare alleles for AMD at or near several complement genes (CFH, C2/CFB, C3, CFI, and C9). Genome-wide association studies (GWAS) identified that a single nucleotide polymorphism (SNP), Y402H, on a gene encoding CFH. The Y402H SNP confers a two to sevenfold increased risk for AMD development (Klein R J et al. Science. 2005; 308:385-9; Edwards A O et al. Science. 2005; 308:421-4; Hageman G S et al. PNAS. 2005; 102:7227-32; Haines J L et al. Science. 2005; 308:419-21). Additional GWAS led to the identification of other variants on the CFH locus which are associated with advanced AMD (Raychaudhuri S et al. Nat Genet. 2011; 43: 1232-6). Overall, these studies have identified that variants near six complement genes (CFH, C2/CFB, C3, CFI, and C9) together accounts for nearly 60% of the AMD genetic risk (Fritsche L G et al. Annu Rev Genomics Hum Genet. 2014; 15:151-71).

Complement system genes (e.g. CFH, FHL-1, FHR1, FHR2, FHR3, FHR4, or FHR5), splice variants (e.g. FHL1), or fragments thereof are provided as transgenes in the recombinant AAV (rAAV) vectors of the disclosure. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell (e.g. an ocular cell). The heterologous nucleic acid sequence (transgene) can be derived from any organism. In certain embodiments, the transgene is derived from a human. In certain embodiments, the transgene encodes a mature form of a complement protein. In some embodiments, the transgene encodes a polypeptide comprising an amino acid sequence that is at least 80%, 85%, 90%, 92%, 95%, or 97% identical to the amino acid sequence of SEQ ID NO: 33, or a biologically active fragment thereof. In some embodiments, the transgene encodes a polypeptide comprising an amino acid sequence that is at least 80%, 85%, 90%, 92%, 95%, or 97% identical to the amino acid sequence of SEQ ID NO: 34, or a biologically active fragment thereof. In certain embodiments, the rAAV vector may comprise one or more transgenes.

In some embodiments, the transgene comprises more than one complement system gene, splice variant, or fragments derived from more than one complement system gene. This may be accomplished using a single vector carrying two or more heterologous sequences, or using two or more rAAV vectors each carrying one or more heterologous sequences. In some embodiments, in addition to a complement system gene, splice variant, or fragment thereof, the rAAV vector may also encode additional proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated subject. The additional proteins, peptides, RNA, enzymes, or catalytic RNAs and the complement factor may be encoded by a single vector carrying two or more heterologous sequences, or using two or more rAAV vectors each carrying one or more heterologous sequences.

In certain aspects, the disclosure provides a recombinant adeno-associated viral (rAAV) vector encoding a human Complement Factor H or Factor H Like 1 (FHL1) protein or biologically active fragment thereof. In certain embodiments, the vector comprises a nucleotide sequence that is at least 80%, 85%, 90%, 92%, 94%, 95%, 97%, 99% or 100% identical to any of the sequences disclosed herein encoding a CFH or CFHL protein, or biologically active fragments thereof. In certain embodiments, the vector comprises a nucleotide sequence that is at least 80%, 85%, 90%, 92%, 94%, 95%, 97%, 99% or 100% identical to any of SEQ ID Nos: 1-3 or 5, or biologically active fragments thereof. In some embodiments, the vector comprises a nucleotide sequence that is at least 70%, 75%, 80%, 85%, 90%, 92%, 94%, 95%, 97%, 99% or 100% identical to SEQ ID NO: 1, or biologically active fragments thereof. In some embodiments, the vector comprises a nucleotide sequence that is at least 70%, 75%, 80%, 85%, 90%, 92%, 94%, 95%, 97%, 99% or 100% identical to SEQ ID NO: 2, or biologically active fragments thereof. In some embodiments, the vector comprises a nucleotide sequence that is at least 70%, 75%, 80%, 85%, 90%, 92%, 94%, 95%, 97%, 99% or 100% identical to SEQ ID NO: 3, or biologically active fragments thereof. In some embodiments, the vector comprises a nucleotide sequence that is at least 70%, 75%, 80%, 85%, 90%, 92%, 94%, 95%, 97%, 99% or 100% identical to SEQ ID NO: 5, or biologically active fragments thereof. In certain embodiments the vector comprises a nucleotide sequence that is at least 80% identical to the nucleotide sequence of SEQ ID NO: 1-3 or 5, or a fragment thereof. In certain embodiments, the nucleotide sequence is at least 90% identical to the nucleotide sequence of SEQ ID NO: 1-3 or 5, or a fragment thereof. In certain embodiments, the nucleotide sequence is at least 95% identical to the nucleotide sequence of SEQ ID NO: 1-3 or 5, or a fragment thereof. In certain embodiments, the nucleotide sequence is the sequence of SEQ ID NO: 1-3 or 5, or a fragment thereof. In certain embodiments, the vector encodes a CFH or an FHL1 protein or biologically active fragment thereof comprising at least four CCP domains. In certain embodiments, the vector encodes CFH or an FHL1 protein or biologically active fragment thereof comprising at least five CCP domains. In certain embodiments, the vector encodes a CFH or an FHL1 protein or biologically active fragment thereof comprising at least six CCP domains. In certain embodiments, the vector encodes a CFH or an FHL1 protein or biologically active fragment thereof comprising at least seven CCP domains. In certain embodiments, the vector encodes an FHL1 protein or biologically active fragment thereof comprising at least three CCP domains. In certain embodiments, the vector encodes a CFH or FHL1 protein or biologically active fragment thereof that comprises at least CCPs 1-2 of CFH. In certain embodiments, the vector encodes a biologically active fragment of CFH that comprises at least CCPs 1-4 of CFH. In certain embodiments, the vector encodes a CFH or FHL1 protein or biologically active fragment thereof that comprises at least CCPs 19-20 of CFH. Schmidt C O, Herbert A P, Kavanagh D, Gandy C, Fenton C J, Blaum B S, Lyon M, Uhrin D, Barlow P N. J Immunol, 2008, 181:2610-9. In certain embodiments, the vector encodes a CFH or an FHL1 protein or biologically active fragment thereof comprising the H402 polymorphism. In certain embodiments, the vector encodes a CFH or an FHL1 protein or biologically active fragment thereof comprising the V62 polymorphism. In certain embodiments, the CFH or FHL1 protein or biologically active fragment thereof comprises the amino acid sequence of SEQ ID NO: 4. In certain embodiments, the amino acid sequence of SEQ ID NO: 4 is the C-terminal sequence of the CFH or FHL1 protein. In certain embodiments, the CFH or FHL1 protein or biologically active fragment thereof is capable of diffusing across the Bruch's membrane. In certain embodiments, the CFH or FHL1 protein or biologically active fragment thereof is capable of binding C3b. In certain embodiments, the CFH or FHL1 protein or biologically active fragment thereof is capable of facilitating the breakdown of C3b.

In certain embodiments, the vector comprises a nucleotide sequence that is at least 80%, 85%, 90%, 92%, 94%, 95%, 97%, 99% or 100% identical to any of SEQ ID Nos: 7, 9, 11, 13, 15, 17, 19, or 21-30, or biologically active fragments thereof.

Exemplary sequences of transgenes are set forth in SEQ ID NOs: 1-3 or 5. In some embodiments, a transgene of the disclosure comprises the nucleic acid sequence set forth in SEQ ID NO: 1. In some embodiments, a transgene of the disclosure comprises the nucleic acid sequence set forth in SEQ ID NO: 2. In some embodiments, a transgene of the disclosure comprises the nucleic acid sequence set forth in SEQ ID NO: 3. In some embodiments, a transgene of the disclosure comprises the nucleic acid sequence set forth in SEQ ID NO: 5. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NOs: 1-3 or 5. One of ordinary skill in the art will appreciate that nucleic acid sequences complementary to the nucleic acids, and variants of the nucleic acids are also within the scope of this disclosure. In further embodiments, the nucleic acid sequences of the disclosure can be isolated, recombinant, and/or fused with a heterologous nucleotide sequence. In some embodiments, any of the nucleotides disclosed herein (e.g., SEQ ID Nos: 1-3 or 5) is codon-optimized (e.g., codon-optimized for human expression)

In one aspect, a transgene encodes a complement system polypeptide with 1, 2, 3, 4, 5, 6, 7, 8, 9, 10, 11, 12, 13, 14, or 15 amino acid substitutions, deletions, and/or additions relative to the wild-type polypeptide. In some embodiments, a transgene encodes a complement system polypeptide with 1, 2, 3, 4, or 5 amino acid deletions relative to the wild-type polypeptide. In some embodiments, a transgene encodes a polypeptide with 1, 2, 3, 4, or 5 amino acid substitutions relative to the wild-type polypeptide. In some embodiments, a transgene encodes a polypeptide with 1, 2, 3, 4, or 5 amino acid insertions relative to the wild-type polypeptide. Polynucleotides complementary to any of the polynucleotide sequences disclosed herein are also encompassed by the present disclosure. Polynucleotides may be single-stranded (coding or antisense) or double-stranded, and may be DNA (genomic or synthetic), cDNA, or RNA molecules. RNA molecules include mRNA molecules. Additional coding or non-coding sequences may, but need not, be present within a polynucleotide of the present disclosure, and a polynucleotide may, but need not, be linked to other molecules and/or support materials.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. The transgenes or variants may also, or alternatively, be substantially homologous to a native gene, or a portion or complement thereof. Such polynucleotide variants are capable of hybridizing under moderately stringent conditions to a naturally occurring DNA sequence encoding a complement factor (or a complementary sequence). Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS. As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in SEQ ID NOs: 1, 2, 3 and 5, or sequences complementary thereto. One of ordinary skill in the art will readily understand that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

The present disclosure further provides oligonucleotides that hybridize to a polynucleotide having the nucleotide sequence set forth in SEQ ID NOs: 1, 2, 3 and 5, or to a polynucleotide molecule having a nucleotide sequence which is the complement of a sequence listed above. Such oligonucleotides are at least about 10 nucleotides in length, and preferably from about 15 to about 30 nucleotides in length, and hybridize to one of the aforementioned polynucleotide molecules under highly stringent conditions, i.e., washing in 6×SSC/0.5% sodium pyrophosphate at about 37° C. for about 14-base oligos, at about 48° C. for about 17-base oligos, at about 55° C. for about 20-base oligos, and at about 60° C. for about 23-base oligos. In a preferred embodiment, the oligonucleotides are complementary to a portion of one of the aforementioned polynucleotide molecules. These oligonucleotides are useful for a variety of purposes including encoding or acting as antisense molecules useful in gene regulation, or as primers in amplification of complement system-encoding polynucleotide molecules.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

The complement system gene or fragment thereof (e.g. a gene encoding CFH, FHL1, FHR1, FHR2, FHR3, FHR4, or FHR5) may be used to correct or ameliorate gene deficiencies, which may include deficiencies in which normal complement system genes are expressed at less than normal levels or deficiencies in which the functional complement system gene product is not expressed. In some embodiments, the transgene sequence encodes a single complement system protein or biologically active fragment thereof. The disclosure further includes using multiple transgenes, e.g., transgenes encoding two or more complement system polypeptides or biologically active fragments thereof. In certain situations, a different transgene may be used to encode different complement proteins or biologically active fragments thereof (e.g. CFH, FHL1, FHR1, FHR2, FHR3, FHR4, or FHR5). Alternatively, different complement proteins (e.g. CFH, FHL1, FHR1, FHR2, FHR3, FHR4, or FHR5) or biologically active fragments thereof may be encoded by the same transgene. In this case, a single transgene includes the DNA encoding each of the complement proteins (e.g. CFH, FHL1, FHR1, FHR2, FHR3, FHR4, or FHR5) or biologically active fragments thereof, with the DNA for each protein or functional fragment thereof separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., MX. Donnelly, et al, J. Gen. Virol, 78(Pt 1): 13-21 (January 1997); Furler, S., et al, Gene Ther., 8(11):864-873 (June 2001); Klump H., et al, Gene Ther., 8(10):811-817 (May 2001). This 2A peptide is significantly smaller than an IRES, making it well suited for use when space is a limiting factor.

The regulatory sequences include conventional control elements which are operably linked to the transgene encoding a complement system polypeptide (e.g. CFH, FHL1, FHR1, FHR2, FHR3, FHR4, or FHR5) or biologically active fragment thereof in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. In some embodiments, the intron comprises the nucleotide sequence of SEQ ID NO: 10, or a codon-optimized or fragment thereof. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In one embodiment, the AAV comprises a promoter (or a functional fragment of a promoter). The selection of the promoter to be employed in the rAAV may be made from among a wide number of promoters that can express the selected transgene in the desired target cell. In one embodiment, the target cell is an ocular cell. In some embodiments, the target cell is a neuronal cell (i.e., the vector targets neuronal cells). However, in particular embodiments, the target cell is a non-neuronal cell (i.e., the vector does not target neuronal cells). In some embodiments, the target cell is a glial cell, Muller cell, and/or retinal pigment epithelial (RPE) cell. The promoter may be derived from any species, including human. In one embodiment, the promoter is “cell specific”. The term “cell-specific” means that the particular promoter selected for the recombinant vector can direct expression of the selected transgene in a particular cell or ocular cell type. In one embodiment, the promoter is specific for expression of the transgene in photoreceptor cells. In another embodiment, the promoter is specific for expression in the rods and/or cones. In another embodiment, the promoter is specific for expression of the transgene in RPE cells. In another embodiment, the promoter is specific for expression of the transgene in ganglion cells. In another embodiment, the promoter is specific for expression of the transgene in Muller cells. In another embodiment, the promoter is specific for expression of the transgene in bipolar cells. In another embodiment, the promoter is specific for expression of the transgene in ON-bipolar cells. In one embodiment, the promoter is metabotropic glutamate receptor 6 (mGluR6) promoter (see, Vardi et al, mGluR6 Transcripts in Non-neuronal Tissues, J Histochem Cytochem. 2011 December; 59(12): 1076-1086, which is incorporated herein by reference). In another embodiment, the promoter is an enhancer-linked mGluR6 promoter. In another embodiment, the promoter is specific for expression of the transgene in OFF-bipolar cells. In another embodiment, the promoter is specific for expression of the transgene in horizontal cells. In another embodiment, the promoter is specific for expression of the transgene in amacrine cells. In another embodiment, the transgene is expressed in any of the above noted ocular cells. In another embodiment, the promoter is the human G-protein-coupled receptor protein kinase 1 (GRK1) promoter (Genbank Accession number AY327580), In another embodiment, the promoter is the human interphotoreceptor retinoid-binding protein proximal (IRBP) promoter.

In some embodiments, the promoter is of a small size, e.g., under 1000 bp, due to the size limitations of the AAV vector. In some embodiments, the promoter is less than 1000, 900, 800, 700, 600, 500, 400 or 300 bp in size. In particular embodiments, the promoter is under 400 bp. In some embodiments, the promoter is a promoter selected from the CRALBP (RLBP), EF1a, HSP70, AAT1, ALB, PCK1, CAG, RPE65, MECP, or sCBA promoter. In some embodiments, the promoter comprises a nucleotide sequence having at least 85%, 90%, 95%, 96%, 97%, 98%, 99%, or 100% identity of any one of SEQ ID NOs: 6, 8, 12, 14, 16, 18, 20, 31, 32, or 36 or codon-optimized and/or fragment thereof. In some embodiments, the promoter comprises the nucleotide sequence of any one of SEQ ID NOs: 6, 8, 12, 14, 16, 18, 20, 31, 32, or 36 or codon-optimized and/or fragment thereof. In some embodiments, the promoter is associated with strong expression in the eye. In some embodiments, the promoter is a CRALBP or RPE65 promoter (e.g., a promoter having the nucleotide sequence of SEQ ID NO: 6 or 32). In some embodiments, the promoter is associated with strong expression in the liver. In some embodiments, the promoter is an AAT1, ALB or PCK1 promoter (e.g., a promoter having the nucleotide sequence of SEQ ID NO: 16, 18, or 20. In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter is less than 1000, 900, 800, 700, 600, 500, 400 or 300 bp in size. In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter is a promoter selected from the CRALBP, EF1a, HSP70 or sCBA promoter. In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter comprises the nucleotide sequence of any one of SEQ ID NOs: 6, 8, 12, 14, 16, 18, 20, 31, 32, or 36 or codon-optimized and/or fragments thereof. In some embodiments, any of the promoters disclosed herein is coupled with a viral intron (e.g., an SV40i intron).

In another embodiment, the promoter is the native promoter for the gene to be expressed. Useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicoud et al, J. Gene Med, December 2007, 9(12): 1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2): 186-94); and the VMD2 promoter (Kachi et al, Human Gene Therapy, 2009 (20:31-9)). Each of these documents is incorporated by reference herein. In one embodiment, the promoter is of a small size, under 1000 bp, due to the size limitations of the AAV vector. In another embodiment, the promoter is under 400 bp.

In certain embodiments, any promoters suitable for use in AAV vectors may be used with the vectors of the disclosure. Examples of suitable promoters include constitutive promoters such as a CMV promoter (optionally with the CMV enhancer), RSV promoter (optionally with the RSV enhancer), SV40 promoter, MoMLV promoter, CB promoter, the dihydrofolate reductase promoter, the chicken β-actin (CBA) promoter, CBA/CAG promoter, and the immediate early CMV enhancer coupled with the CBA promoter, or a EF1a promoter, etc. In some embodiments a cell- or tissue-specific promoter is utilized (e.g., a rod, cone, or ganglia derived promoter). In certain embodiments, the promoter is small enough to be compatible with the disclosed constructs, e.g., the CB promoter. Preferably, the promoter is a constitutive promoter. In another embodiment, the promoter is cell-specific. The term “cell-specific” means that the particular promoter selected for the recombinant vector can direct expression of the selected transgene in a particular ocular cell type. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In another embodiment, the promoter is specific for expression of the transgene in RPE cells. In another embodiment, the transgene is expressed in any of the above noted ocular cells.

Other useful promoters include transcription factor promoters including, without limitation, promoters for the neural retina leucine zipper (Nrl), photoreceptor-specific nuclear receptor Nr2e3, and basic-leucine zipper (bZIP). In one embodiment, the promoter is of a small size, under 1000 bp, due to the size limitations of the AAV vector. In another embodiment, the promoter is under 400 bp.

Other regulatory sequences useful herein include enhancer sequences Enhancer sequences useful herein include the IRBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). It is understood that not all vectors and expression control sequences will function equally well to express all of the transgenes as described herein. However, one of skill in the art may make a selection among these, and other, expression control sequences to generate the rAAV vectors of the disclosure.

Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997). Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a transgene encoding a complement system polypeptide (e.g. CFH, FHL1, FHR1, FHR2, FHR3, FHR4, or FHR5) or a biologically active fragment thereof) flanked by at least one AAV ITR sequence; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The rAAV particles can be produced using methods known in the art. See, e.g., U.S. Pat. Nos. 6,566,118; 6,989,264; and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Recombinant AAV particles are generated by transfecting producer cells with a plasmid (cis-plasmid) containing a rAAV genome comprising a transgene flanked by the 145 nucleotide-long AAV ITRs and a separate construct expressing the AAV rep and CAP genes in trans. In addition, adenovirus helper factors such as E1A, E1B, E2A, E4ORF6 and VA RNAs, etc. may be provided by either adenovirus infection or by transfecting a third plasmid providing adenovirus helper genes into the producer cells. Producer cells may be HEK293 cells. Packaging cell lines suitable for producing adeno-associated viral vectors may be readily accomplished given readily available techniques (see e.g., U.S. Pat. No. 5,872,005). The helper factors provided will vary depending on the producer cells used and whether the producer cells already carry some of these helper factors.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified.

In some embodiments, rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a therapeutic polypeptide and/or nucleic acid as described herein flanked by at least one AAV ITR, and (iii) an AAV helper function; and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV 12, AAV2R471A, AAV DJ, a goat AAV, bovine AAV, or mouse AAV or the like. In some embodiments, the encapsidation protein is an AAV2 encapsidation protein.

Suitable rAAV production culture media of the present disclosure may be supplemented with serum or serum-derived recombinant proteins at a level of 0.5-20 (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2μη Filter Opticap XL 10 Millipore Express SHC Hydrophilic Membrane filter. Clarification can also be achieved by a variety of other standard techniques known in the art, such as, centrifugation or filtration through any cellulose acetate filter of 0.2μη or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

Pharmaceutical Compositions

Also provided herein are pharmaceutical compositions comprising an rAAV particle comprising a transgene encoding a complement system polypeptide (e.g. CFH, FHL1, FHR1, FHR2, FHR3, FHR4, or FHR5) or a biologically active fragment thereof and/or therapeutic nucleic acid, and a pharmaceutically acceptable carrier. The pharmaceutical compositions may be suitable for any mode of administration described herein; for example, by intravitreal administration.

In some embodiments, the composition comprises a polypeptide (or a nucleic acid encoding a polypeptide) that processes (e.g., cleaves) the complement system polypeptide encoded by the transgene in the rAAV. However, in particular embodiments, the composition does not comprise a polypeptide (or a nucleic acid encoding a polypeptide) that processes (e.g., cleaves) the complement system polypeptide encoded by the transgene in the rAAV.

Gene therapy protocols for retinal diseases, such as LCA, retinitis pigmentosa, and age-related macular degeneration require the localized delivery of the vector to the cells in the retina. The cells that will be the treatment target in these diseases are either the photoreceptor cells in the retina or the cells of the RPE underlying the neurosensory retina. Delivering gene therapy vectors to these cells requires injection into the subretinal space between the retina and the RPE. In some embodiments, the disclosure provides methods to deliver rAAV gene therapy vectors encoding a complement system polypeptide (e.g. CFH, FHL1, FHR1, FHR2, FHR3, FHR4, or FHR5) or a biologically active fragment thereof to cells of the retina.

In some embodiments, the pharmaceutical compositions comprising a rAAV described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising a rAAV described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the recombinant AAV containing the desired transgene encoding a complement system polypeptide (e.g. CFH, FHL1, FHR1, FHR2, FHR3, FHR4, or FHR5) or a biologically active fragment thereof and constitutive or tissue or cell-specific promoter for use in the target ocular cells as detailed above is formulated into a pharmaceutical composition intended for subretinal or intravitreal injection. In some embodiments, the compositions disclosed herein targets cells of any one or more regions of the macula including, for example, the umbo, the foveolar, the foveal avascular zone, the fovea, the parafovea, or the perifovea. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired. In certain embodiments, the pharmaceutical compositions of the disclosure are administered after administration of an initial loading dose of the complement system protein.

In some embodiments, any of the vectors/pharmaceutical compositions disclosed herein are administered to a patient such that they target glial cells, Muller cells, and/or retinal pigment epithelial cells. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the vector/composition is to be administered to an elderly adult (e.g., at least 60 years of age). In particular embodiments, any of the vectors/pharmaceutical compositions disclosed herein are administered to a subject intravitreally. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues {e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in International Patent Application No. PCT/US2013/022628. In view of the imaging and functional studies, in some embodiments, one or more injections are performed in the same eye in order to target different areas of retained bipolar cells. The volume and viral titer of each injection is determined individually, as further described below, and may be the same or different from other injections performed in the same, or contralateral, eye. In another embodiment, a single, larger volume injection is made in order to treat the entire eye. In one embodiment, the volume and concentration of the rAAV composition is selected so that only a specific region of ocular cells is impacted. In another embodiment, the volume and/or concentration of the rAAV composition is a greater amount, in order reach larger portions of the eye, including non-damaged ocular cells.

The composition may be delivered in a volume of from about 0.1 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. An effective concentration of a recombinant adeno-associated virus carrying a nucleic acid sequence encoding the desired transgene under the control of the cell-specific promoter sequence desirably ranges from about 10⁷ and 10¹³ vector genomes per milliliter (vg/mL) (also called genome copies/mL (GC/mL)). The rAAV infectious units are measured as described in S. K. McLaughlin et al, 1988 J. Virol., 62: 1963, which is incorporated herein by reference. Preferably, the concentration in the retina is from about 1.5×10⁹ vg/mL to about 1.5×10¹² vg/mL, and more preferably from about 1.5×10⁹ vg/mL to about 1.5×10¹¹ vg/mL. In certain preferred embodiments, the effective concentration is about 2.5×10¹⁰ vg to about 1.4×10¹¹. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. In one embodiment, the effective dosage (total genome copies delivered) is from about 10⁷ to 10¹³ vector genomes. It is desirable that the lowest effective concentration of virus be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina. Intravenous delivery, for example may require doses on the order of 1.5×10¹³ vg/kg.

Pharmaceutical compositions useful in the methods of the disclosure are further described in PCT publication No. WO2015168666 and PCT publication no. WO2014011210, the contents of which are incorporated by reference herein.

Methods of Treatment/Prophylaxis

Described herein are various methods of preventing, treating, arresting progression of or ameliorating the ocular disorders and retinal changes associated therewith. Generally, the methods include administering to a mammalian subject in need thereof, an effective amount of a composition comprising a recombinant adeno-associated virus (AAV) described above, carrying a transgene encoding a complement system polypeptide (e.g. CFH, FHL1, FHR1, FHR2, FHR3, FHR4, or FHR5) or a biologically active fragment thereof under the control of regulatory sequences which express the product of the gene in the subject's ocular cells, and a pharmaceutically acceptable carrier. Any of the AAV described herein are useful in the methods described below.

Gene therapy protocols for retinal diseases, such as LCA, retinitis pigmentosa, and age-related macular degeneration require the localized delivery of the vector to the cells in the retina. The cells that will be the treatment target in these diseases are either the photoreceptor cells in the retina or the cells of the RPE underlying the neurosensory retina. Delivering gene therapy vectors to these cells requires injection into the subretinal space between the retina and the RPE. In some embodiments, the disclosure provides methods to deliver rAAV gene therapy vectors comprising a complement system gene or a fragment thereof to cells of the retina.

In a certain aspect, the disclosure provides a method of treating a subject having age-related macular degeneration (AMD), comprising the step of administering to the subject any of the vectors of the disclosure. In certain embodiments, the vectors are administered at a dose between 2.5×10¹⁰ vg and 1.4×10¹¹ vg/per eye in about 50 μl to about 100 μl. In certain embodiments, the vectors are administered at a dose between 1.0×10¹¹ vg and 1.5×10¹³ vg/per eye in about 50 μl to about 100 In certain embodiments, the vectors are administered at a dose between 1.0×10¹¹ vg and 1.5×10¹² vg/per eye in about 50 μl to about 100 In certain embodiments, the vectors are administered at a dose of about 1.4×10¹² vg/per eye in about 50 μl to about 100 In certain embodiments, the vectors are administered at a dose of 1.4×10¹² vg/per eye in about 50 μl to about 100 In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the vectors are administered by intravitreal injection in a solution of PBS with additional NaCl and pluronic.

In some embodiments, any of the vectors of the present disclosure used according to the methods disclosed herein is capable of inducing at least 5%, 10%, 20%, 50%, 100%, 150%, 200%, 250%, 300%, 400%, 500%, 700%, 900%, 1000%, 1100%, 1500%, or 2000% higher expression of CFH and/or FHL1 in a target cell disclosed herein (e.g., an RPE or liver cell) as compared to the endogenous expression of CFH and/or FHL1 in the target cell. In some embodiments, expression of any of the vectors disclosed herein in a target cell disclosed herein (e.g., an RPE or liver cell) results in at least 5%, 10%, 20%, 50%, 100%, 150%, 200%, 250%, 300%, 400%, 500%, 700%, 900%, 1000%, 1100%, 1500%, or 2000% higher levels of CFH and/or FHL1 activity in the target cell as compared to endogenous levels of CFH and/or FHL1 activity in the target cell.

In some embodiments, any of the vectors disclosed herein is administered to cell(s) or tissue(s) in a test subject. In some embodiments, the cell(s) or tissue(s) in the test subject express less CFH and/or FHL1, or less functional CFH and/or FHL1, than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye and/or a specific cell type of the eye (e.g., cells in the foveal region) in the test subject express at least 95%, 90%, 80%, 70%, 60%, 50%, 40%, 30%, 20%, 10%, 5%, or 1% less CFH and/or FHL1 or functional CFH and/or FHL1 as compared to the levels in the reference control subject or population of reference control subjects. In some embodiments, a the eye or a specific cell type of the eye (e.g., cells in the foveal region) in the test subject express CFH and/or FHL1 protein having any of the CFH and/or FHL1 mutations disclosed herein. In some embodiments, the eye or a specific cell type of the eye (e.g., cells in the foveal region) in the reference control subject do not express a CFH and/or FHL1 protein having any of the CFH and/or FHL1 mutations disclosed herein. In some embodiments, expression of any of the vectors disclosed herein in the cell(s) or tissue(s) of the test subject results in an increase in levels of CFH and/or FHL1 protein or functional CFH and/or FHL1 protein. In some embodiments, expression of any of the vectors disclosed herein in the cell(s) or tissue(s) of the test subject results in an increase in levels of CFH and/or FHL1 protein or functional CFH and/or FHL1 protein such that the increased levels are within 90%, 80%, 70%, 60%, 50%, 40%, 30%, 20%, 10%, 5%, or 1% of, or are the same as, the levels of CFH and/or FHL1 protein or functional CFH and/or FHL1 protein expressed by the same cell type or tissue type in the reference control subject or population of reference control subjects. In some embodiments, expression of any of the vectors disclosed herein in the cell(s) or tissue(s) of the test subject results in an increase in levels of CFH and/or FHL1 protein or functional CFH and/or FHL1 protein, but the increased levels of CFH and/or FHL1 protein or functional CFH and/or FHL1 protein do not exceed the levels of CFH and/or FHL1 protein or functional CFH and/or FHL1 protein expressed by the same cell type or tissue type in the reference control subject or population of reference control subjects. In some embodiments, expression of any of the vectors disclosed herein in the cell(s) or tissue(s) of the test subject results in an increase in levels of CFH and/or FHL1 protein or functional CFH and/or FHL1 protein, but the increased levels of CFH and/or FHL1 protein or functional CFH and/or FHL1 protein exceed the levels of CFH and/or FHL1 protein or functional CFH and/or FHL1 protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects. In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more mutations that causes amacular degeneration (AMD) or that increases the likelihood that a patient develops AMD. In some embodiments, any of the treatment and/or prophylactic methods disclosed herein is for use in treatment of a patient having one or more mutations that causes atypical hemolytic uremic syndrome (aHUS) or that increases the likelihood that a patient develops aHUS. In some embodiments, the one or more mutations are in the patient's CFI gene. In some embodiments, the one or more mutations are in the patient's CFH gene. In some embodiments, the one or more mutations are in both the patient's CFH and CFI genes. In some embodiments, the subject has a loss-of-function mutation in the subject's CFH gene. In some embodiments, the subject has a loss-of-function mutation in the subject's CFI gene.

In some embodiments, any of the treatment and/or prophylactic methods disclosed herein is for use in treatment of a patient having one or more mutations in the patient's CFI gene. In some embodiments, the patient has a mutation in one or more of the FIMAC, CD5, L1, L1-Ca binding, L1-disulfid bond, L2, L2-Ca binding, serine protease, or serine protease active site domains. In some embodiments, the patient has one or more mutations in the disulphide bond sites in the CFI protein. In some embodiments, the mutation is one or more of the mutations selected from the group consisting of: E548Q, V412M, A431T, A431S, K441R, P553S, A240G, A258T, G119R, G261D, R2021, T300A, T2031, V152M, R317W, G287R, E554V, 1340T, G162D, P50A, Y206N, D310E, H418L, p.(Tyr411Stop), p.(Arg187Stop), R474Q, Y459S, R187Q, R339Q, G263V, p.(Arg339Stop), D477H, p.(Ile357Met), P64L, E109A, G125R, N1771, F198L, S221Y, D224N, C229R, V230M, G248E, G280D, A356P, V201, Y369S, W374C, R389H, W399R, C467R, G487C, I492L, G500R, R502C, W541*, V543A, Q580*, V355M, I578T, R474*, R406H, D44N, p.(Arg406Cys), D403N, 1416L, G328R, G512S, p.(Gly542Ser), p.(Cys106Arg), V127A, p.(Ile55Phe), H40R, C54R, C54*, V184M, G362A, Q462H, N536K, R317Q, p.(His 183Arg), p.(Ile306Val), p.(Gly342Glu), p.(Asp429Glu), R448H, D519N, S493R, R448C, K338Q, G104R, C259R, G372S, A360V, E290A, V213F, F13V, Y514Ter, V396A, E303Q, H401Q, 1306T, E479G, c.772+1G>T, F498L, Y411H, S24T, C255Y, R168S, Q228R, V4691, Q250K, Y241C, G232V, G248R, G110R, E109K, N422D, C550R, G242AfsTer9, R345G, N428MfsTer5, C550WfsTer17, V341E, N428S, H334P, W51R, A452S, T72S, T72S, V5581, E445G, C444Y, L3511, G261S, M1381, A563S, G263AfsTer37, K142E, c.658+2T>C, G205D, T197A, G188V, A378V, L376P, C365Y, M147V, Q161Ter, G439R, G269S, R201S, P576S, Y65H, c.907+1G>Aâ€, Y22C, 1407T, M204V, A384T, G516V, R336G, F139V, L4H, K117E, V4891, P402L, G547R, A346T, S326P, I126T, D283G, S298F, loss of Metl, Ter584QextTer24, C521Y, R168G, S457P, A423E, L34V, A452T, K442E, N245K, D173N, K267E, S146R, E302K, G295V, V299L, K111N, S113N, F17V, Q391E, H14L, T3941, c.659-2A>G, A511V, E303K, D398G, Ter584KextTer24, V583A, A163T, H118Q, A309S, T231, G473R, V5301, E26Ter, K497N, S496C, S496T, L491R, V412E, F417S, S570G, D465G, E124K, D567V, G557D, E548G, W546G, V5431, N464K, P463A, N564S, K561E, E445D, C444G, D443H, E434KfsTer2, 1430T, I244S, I244V, c328+1G>A, R345Q, S175F, N331KfsTer46, C327R, K1301, Q260E, P96S, 1140T, T1371, D135G, K69E, G57D, G371V, G367A, N279S, Y276C, G269C, E190D, T300A, G261D, N151S, R406H, V152M, G362A, E554V, S570T, 1340T, K441R, T2031, Y206N, G328R, T107A, P553S, G287R, N70T, P50A, R406C, R187Q, G119R, 0.1429+1G>C, D477H, N1771, V129A, I55V, W399R, G500R, I492L, R339Ter, I357M, R474Q, D44N, D403N, R474Ter, R317W, G512S, R339Q, A356P, R187Ter, 1416L, R317L, R389H, 1306V, D224Y, R317Q, A258T, Q580Tet, H418L, I578T, G542S, P64L, C106R, Y369S, Q462H, A240G, H183R, R502G, H40R or G162D. In particular embodiments, the mutation is any one of the mutations selected from the group consisting of: G119R, L131R, V152M, G162D, R187Y, R187T, T2031, A240G, A258T, G287R, A300T, R317W, R339Q, V412M, and P553S. In some embodiments, any of the CFI mutant amino acid positions described herein correspond to the wildtype amino acid CFI sequence of SEQ ID NO: 35.

In some embodiments, any of the treatment and/or prophylactic methods disclosed herein is for use in treatment of a patient having one or more mutations in the patient's CFH gene. In some embodiments, the patient has a mutation in one or more of the pre-SCR1 or any of the SCR1-SCR20 domains. In some embodiments, the patient has a mutation in one or more of the transition regions between SCRs. In some embodiments, the mutation is one or more of the mutations selected from the group consisting of: H402Y, G69E, D194N, W314C, A806T, Q950H, p. I1e184fsX, p.Lys204fsX, c.1697-17_-8del, A161S, A173G, R175Q, V62I, V1007L, S890I, S193L, I216T, A301Nfs*25, W379R, Q400K, Q950H, T956M, R1210C, N1050Y, E936D, Q408X, R1078S, c.350+6T->G, R567G, R53C, R53H, R2T, A892V, R567G, I221V, S159N, P562H, F960S, R303W, R303Q, K666N, G1194D, P258L, G650V, D130N, S58A, R166W, R232Q, R127H, K1202N, G397Stop, Stop450R, R830W, I622L, T732M, S884Y, L24V, Y235H, K527N, R582H, C973Y, V1089M, E123G, T291S, R567K, E625Stop, N802S, N1056K, R1203W, Q1076E, P26S, T46A, T91S, C129Y, R166Q, E167Q, R175P, C192F, W198*, V206M, G218*, M239T, Y277*, C325Y, R341H, R364L, P384R, C431S, D454A, A473V, P503A, N516K, I551T, H699R, F717L, W978R, P981S, A1010V, W1037*, P1051L, I1059T, Q1143E, R1206H, T12271, L24V, H169R, R257H, K410E, V6091, D619N, A892V, G1002R, G278S, T30*, I32Stop, R78G, Q81P, V111E, W134R, P139S, M162V, E189Stop, K224Del, K224Del, A307A, H332Y, S411T, C448Y, L479Stop, R518T, T519A, C536R, C564P, C569Stop, L578Stop, P621T, C623S, C630W, E635D, K670T, Q672Q, C673Y, C673S, S714Stop, S722*, C733Y, V737V, E762Stop, N774Stop, R7801, G786*, M823T, V835L, E847V, E850K, C853R, C853T, C864S, C870R, H878H, I881L, E889Stop, H893R, Y899Stop, Y899D, C915S, C915Stop, W920R, Q925Stop, C926F, Y951H, C959Y, P968*, 1970V, T987A, N997T, G1011*, T10171, Y1021F, C1043R, T1046T, V1054I, V1060A, V1060L, C1077W, T1097W, T1097T, D1119G, D1119N, P1130L, V1134G, E1135R, E1137L, E1139Stop, Y1142D, Y1142C, C1152S, W1157R, P1161T, C1163T, P1166L, V1168E, V1168Stop, I1169L, E1172Stop, Y1177C, R1182S, W1183L, W1183R, W1183L, W1183Stop, W1183C, T1184R, T1184A, K1186H, K1188Del, L1189R, L1189F, S1191L, S1191W, E1195Stop, V1197A, E1198A, E1198Stop, F1199S, V1200L, G1204E, L1207R, S1211P, R1215Q, R1215G, T1216Del, C1218R, Y1225*, P1226S, L3V, H821Y, E954del, G255E, T1038R, V383A, V641A, P213A, I221V, E229K, R2T, R1072G, G967E, N819S, V579F, G19K, A18S, K834E, 1504M, R6621, P668L, G133R, I184T, L697F, H1165Y, G1110A, pI1e808_Gln809del, I760L, T447R, I808M, I868M, L765F, N767S, R567G, K768N, S209L, Q628K, D214Y, N401D, I216K, Q464R, I777V, E229D, M8231, R232Ter, S266L, P260S, E23G, C80Y, R781, R582H, N638D, N638S, P258L, L3F, R257H, G240R, G69R, D855N, M111, K472N, Q840H, E850K, Y899H, T645M, M805V, K919T, E201G, V407A, 1907L, T914K, H332R, V144M, S652G, D195N, C146S, P661R, E677Q, V482I, I34R, A421T, R281G, C509Y, K666N, P440S, C442G, N607D, A425V, G667E, P440L, I49V, R387G, E625K, E625Ter, T135S, P43S, K283E, I124V, T36V, 15631, G350E, D619G, T321I, T286A, P384L, T739N, M515L, V158A, G727R, T724K, F717L, M162V, C178R, G700R, A161T, F176L, R295S, F298Y, G297S, P300L, R1040K, V552L, T310I, T531A, G928D, Ter386RextTer 69â€, Q1143K, Y534C, P981L, K308N, D538E, R1215Ter, E105V, T10171, N10501, P935S, Y951H, T1097M, D947H, E961D, G962S, G964E, 1970V, R1072T, P1114L, S1122T, F960C, R1074C, R1182T, R1074L, S884Y, S890T, V8371, V941F, V1581, D748V, I216T, H371N, L750F, P418T, M432V, D693N, A746E, V111E, c.2237-2A>G, P982S, V579A, E591D, V5791, V651, P418S, Y1067C, D772N, V72L, E189K, A1027P, D798N, N61D, P384S, N521S, P1068S, E395K, N774S, H577R, E833K, K6E, H337R, R444C, L741F, Y42F, D288E, S705F, R1040G, D214H, N757D, I861M, G848E, P923S, E201K, E902A, R303Q, G366E, D538H, K82R, E721K, Y1008H, R1074P, A806S, Q807R, C389Y, H764Y, K867N, P392T, L394M, E456K, F459L, Y398C, E570K, D214N, I574V, I574T, G631C, T8801, V865F, V576A, N776S, P633S, N22D, P634A, N8221, R885S, R232L, E635D, R778K, L827V, C267R, Y779C, R582C, L77S, R257C, Y327H, N75K, L74F, S836T, Y243H, c.1519+5_1519+8delGT . . . , K507Q, A892S, I15T, P924L, A14V, N842K, G894R, G894E, Y271C, C9W, T504R, V683M, L385Pheâ€, S898R, Q408H, G409S, T34K, E648G, I412V, E338D, P799S, G480E, D798E, D195Y, R341C, D485H, D485G, K598Q, Y420H, P599T, N434H, R441T, C431G, V149A, V3491, T679A, P43T, G45D, R662G, T5191, L121P, P364L, P621A, H373Y, D538MfsTer14, H371P, T544A, T131A, R166G, V1771, V177A, R729S, F717V, N718S, S991G, L981, Y1016Ter, T1217del, M1001T, K1004E, A1010T, G1011D, T1017A, T1031A, L1125F, R1203G, L1214M, W1096DfsTer20, H939N F960L, D966H, M10641, E1071K, N1095K, T1106A, G1107E, C1109W, P1111S, V11971, Y1075F, S1079N, P1080S, E1082G, or Sto1232. In particular embodiments, the mutation is one or more of the mutations selected from the group consisting of: R2T, L3V, R53C, R53H, S58A, G69E, D90G, R175Q, S193L, I216T, 1221V, R303W, H402Y, Q408X, P503A, G650V, R1078S, and R1210C. In some embodiments, any of the CFH mutant amino acid positions described herein correspond to the wildtype amino acid CFH sequence of SEQ ID NO: 33.

In some embodiments, any of the vectors disclosed herein are for use in treating a renal disease or complication. In some embodiments, the renal disease or complication is associated with AMD in the patient. In some embodiments, the renal disease or complication is associated with aHUS in the patient. In some embodiments, the vector administered for treating a renal disease or complication comprises a promoter that is associated with strong expression in the liver. In some embodiments, the promoter is an AAT1, PCK1, or ALB1 promoter (e.g., a promoter comprising the nucleotide sequence of any one of SEQ ID Nos: 16, 18 or 20).

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of the rAAV described above.

In some embodiments, any of the vectors or compositions disclosed herein is administered to a subject in combination with any of the other vectors or compositions disclosed herein. In some embodiments, any of the vectors or compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

In some embodiments, any of the vectors disclosed herein is administered to a subject in combination with an additional agent needed for processing and/or improving the function of the protein encoded by the vector/composition. For example, if the vector comprises a CFH gene, the vector may be administered to a patient in combination with an antibody (or a vector encoding that antibody) that potentiates the activity of the expressed CFH protein. Examples of such antibodies are found in WO2016/028150, which is incorporated herein in its entirety. In some embodiments, the vector is administered in combination with an additional polypeptide (or a vector encoding that additional polypeptide), wherein the additional polypeptide is capable of processing the protein encoded by the vector, e.g., processing an encoded precursor protein into its mature form. In some embodiments, the processing protein is a protease (e.g., a furin protease).

Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1: Construction of AAV Vectors

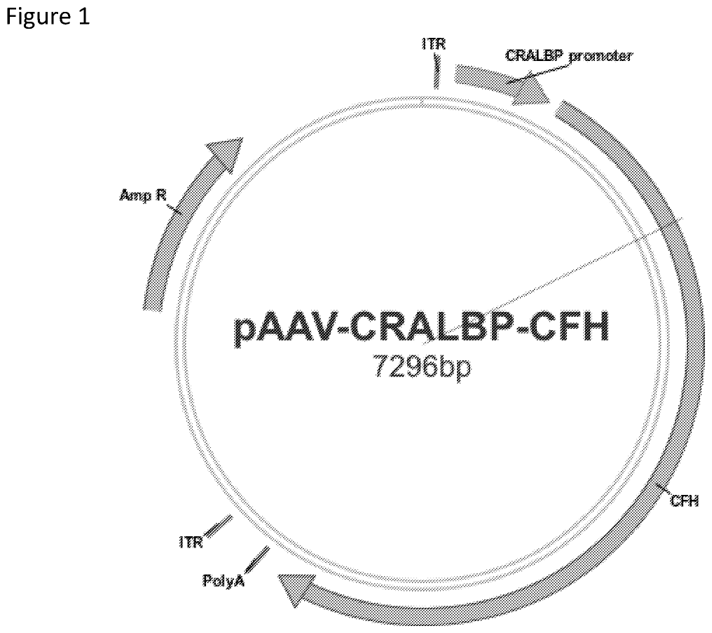

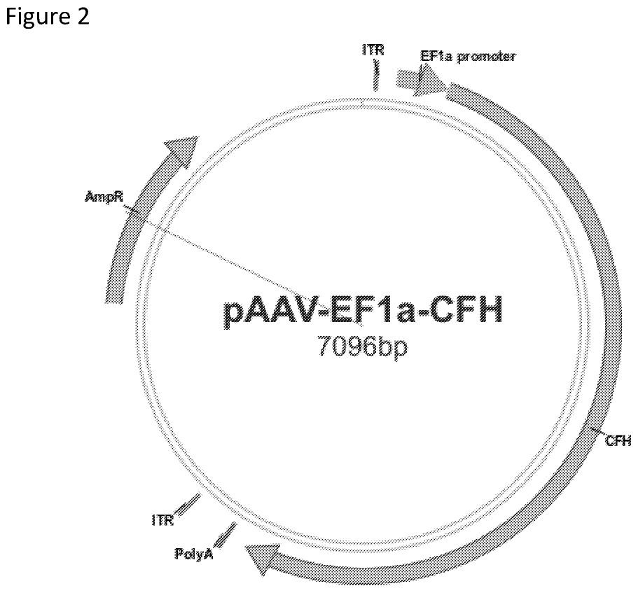

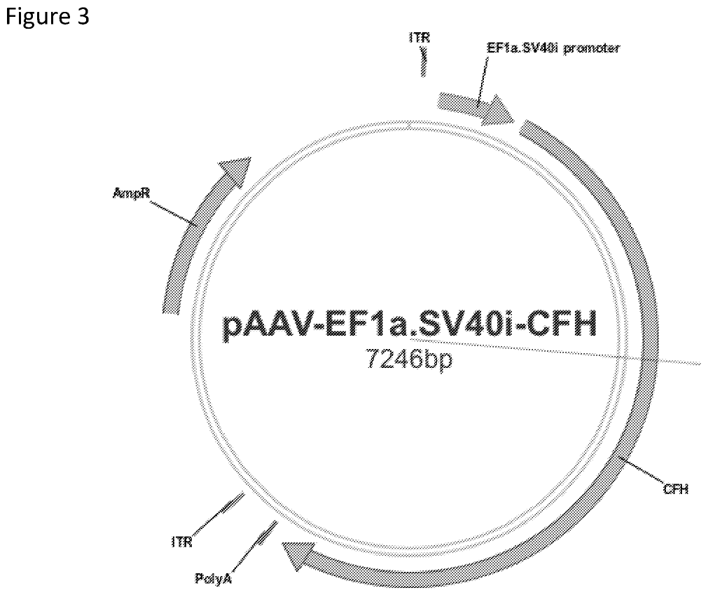

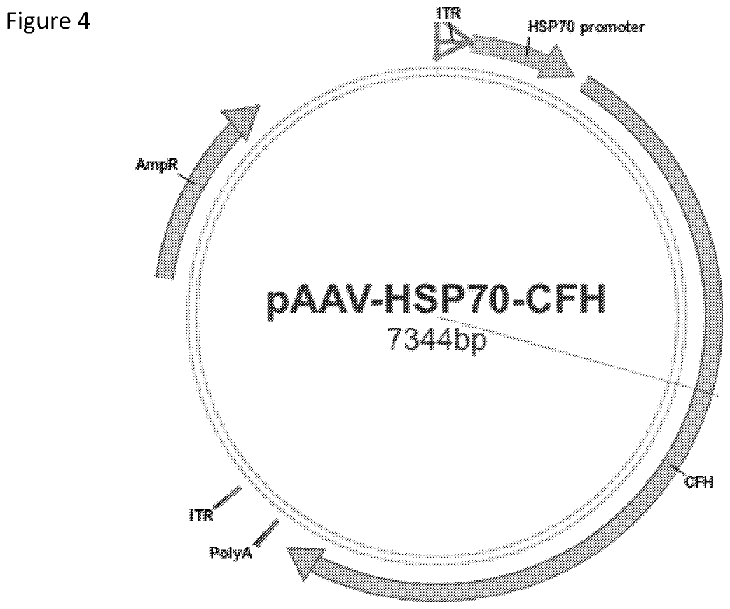

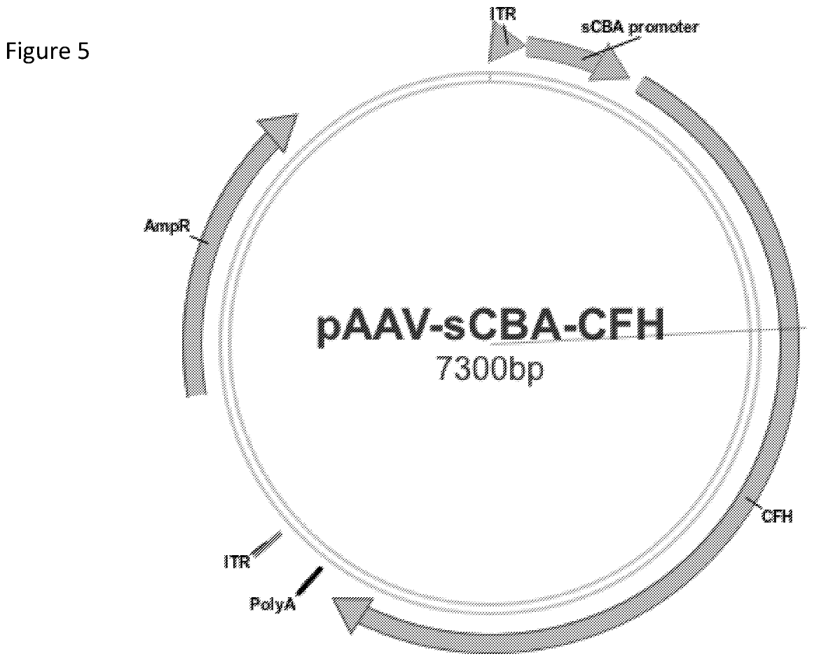

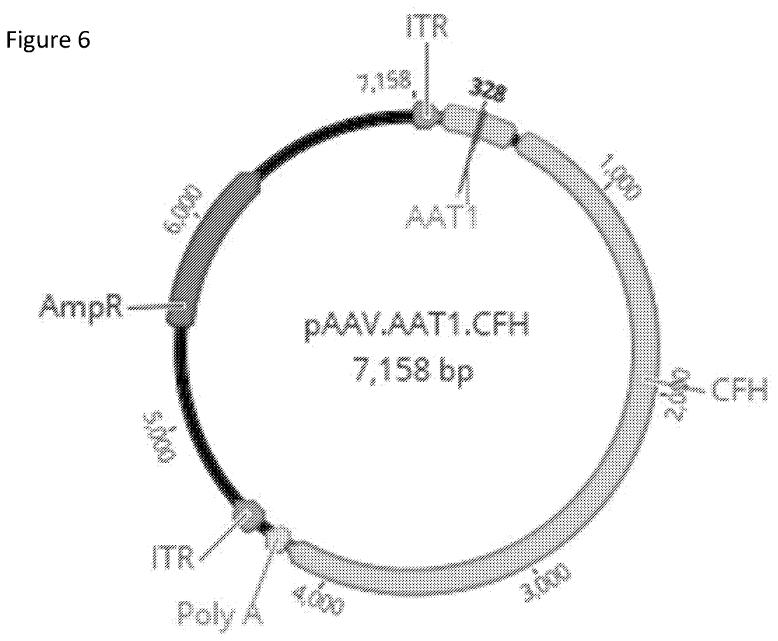

AAV2 vectors were designed comprising either codon-optimized or non-codon-optimized CFH and/or CFHL sequences in combination with a variety of different promoters and, in some cases, SV40 introns. FIGS. 1-6 show vector maps of the different vectors generated. A table is provided below outlining the gene included in the cassette, the promoter included, the Figure laying out the construct map, and the sequence associated with the vector.

| | | | | Construct |
|---|---|---|---|---|
| Construct Name | Transgene | Promoter | Figure | Sequence |
| pAAV-CRALBP- | CFH | CRALBP | 1 | 7 |
| CFH | | | | |
| pAAV-EF1a-CFH | CFH | EF1a | 2 | 9 |
| pAAV-EF1a- | CFH | EF1a | 3 | 11 |
| SV40i-CFH | | | | |
| pAAV-HSP70- | CFH | HSP70 | 4 | 13 |
| CFH | | | | |
| pAAV-sCBA-CFH | CFH | CBA | 5 | 15 |
| pAAV-AAT1-CFH | CFH | AAT1 | 6 | 17 |
| pAAV-ALB-CFH | CFH | ALB | 7 | 19 |
| pAAV-PCK1-CFH | CFH | PCK1 | 8 | 21 |
| pAAV-EF1a- | CFHL | EF1a | 9 | 22 |
| CFHL | | | | |
| pAAV-ALB-CFHL | CFHL | ALB | 10 | 23 |
| pAAV-AAT1- | CFHL | AAT1 | 11 | 24 |
| CFHL | | | | |
| pAAV-EF1a- | CFHL | EF1a | 12 | 25 |
| SV40i-CFHL | | | | |
| pAAV-CAG- | CFHL | CAG | 13 | 26 |
| CFHL | | | | |
| pAAV-CRALBP- | CFHL | CRALBP | 14 | 27 |
| CFHL | | | | |
| pAAV-hRPE65- | CFHL | hRPE65 | 15 | 28 |
| CFHL | | | | |
| pAAV-HSP70- | CFHL | HSP70 | 16 | 29 |
| CFHL | | | | |
| pAAV-PCK1- | CFHL | PCK1 | 17 | 30 |
| CFHL | | | | |

Ability of AAV.CFH and AAV.FHL1 Vectors to Transduce Cells and Regulate Complement Activity:

The CFH vectors indicated above each will be first tested in vitro in HEK293 and ARPE19 cells via transfection and evaluated for expression of the human CFH and FHL1 protein in both cell pellets and in the supernatant. Techniques like Western blot will be used for protein detection and quantification. Quantitative Real time PCR will be used for determining mRNA expression levels. Regulation of complement activity will be tested in a cell culture model of blue light irradiation of A2E-laden retinal pigment epithelial cells as described in van der Burght et al, Acta Ophthalmol, 2013. Briefly, ARPE-19 cell line is grown to confluence and cultured in standard media plus or minus 10 uM A2E for 4 weeks. RPE are irradiated with blue light. Media is replaced with PBS plus calcium, magnesium and 5.5 mM glucose and cells are irradiated with blue light (430+/−30 nm) for 0, 5 or 10 minutes. RPE cells are incubated with appropriately-complement depleted human serum+/−and transfected with the AAV.CFH and AAV.FHL1 vectors. Immunoreactivity of RPE with cell surface markers, CD46, CD55 and CD59 and C3 and MAC deposition will be assessed by fluorescent microscopy or western blot. Levels of iC3b will be measured by Western Blot.

After evaluation in ARPE19 cells, the AAV.CFH and AAV.FHL1 vectors will be tested in mouse models of light-induced retinal degeneration and laser induced choroidal neovascularization via intravitreal injections. Amount of protein produced and its biodistribution in the retina will be tested via Western blot and immunohistochemistry. Rescue of photoreceptor thinning and RPE cell death will be assessed via optical coherence tomography, fundus photography and histological analyses. Immunoreactivity of RPE with cell surface markers, CD46, CD55 and CD59 and C3 and MAC deposition will be assessed by fluorescent microscopy or western blot. Levels of iC3b (cleavage product of C3) will be measured by Western Blot.

Appropriate dose for non-human primates will be determined based on mouse studies. Non-human primate studies will be conducted in cynomolgus monkeys via intravitreal injections. Therapeutic benefits will be evaluated based on levels of CFH and FHL1 proteins produced and secreted by the RPE. Amount of secreted CFH and FHL1 protein will be measured in the retina and the choroid compared to uninjected or sham injected cohorts. Increased levels of CFH and FHL1 in the retina and choroid is expected to provide therapeutic benefits in the AMD population with rare mutations that lead to the loss or decreased amount of these protein.

### Example 2: Transfection of HEK-293T Cells with CFH Plasmids

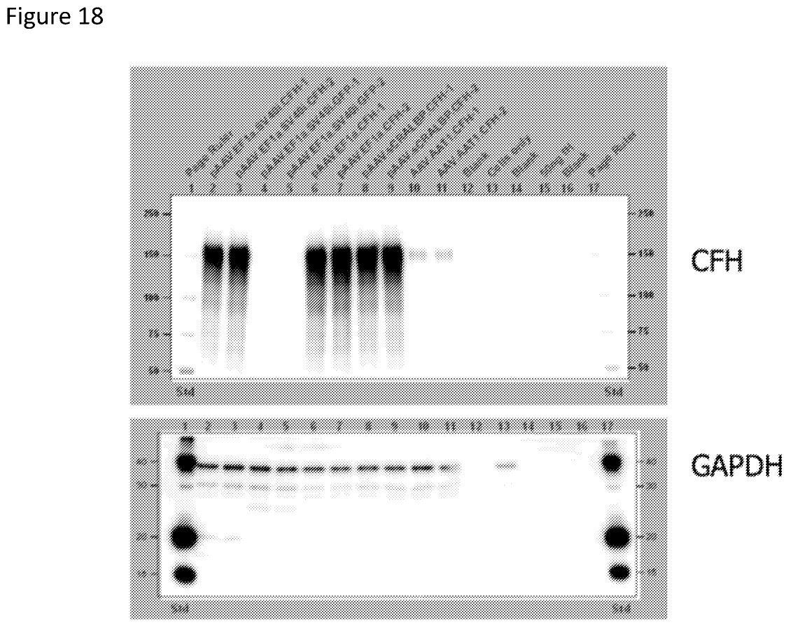

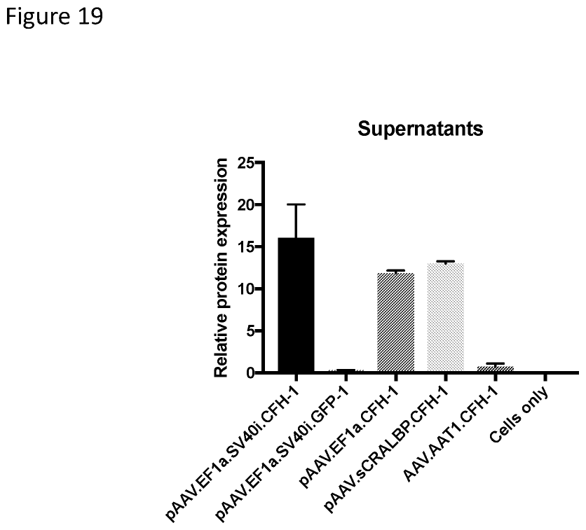

Plasmids capable of expressing CFH or GFP under the control of one of several specific promoters (EF1a.SV40i; EF1a; CRALBP; or AAT1) were transfected into HEK-293T cells. Cells were transfected using 1 mg/L plasmid DNA. Cells were transfected with PEI at a 1:1 DNA:PEI ratio. Cells were cultured for 120 hr and sampled for analysis. Cells were lysed and supernatants were harvested and run on reducing PAGE gel and transferred to membranes for Western blot. Primary antibody for detection of CFH is Quidel goat antiserum to human CFH at 1:1000 at 4° C. with rotation 0/N. Secondary antibody was rabbit anti-goat at 1:5000 for 1 hour at room temperature. Rabbit anti-GAPDH polyclonal antibody is included for loading control (1:1000 dilution) and the secondary antibody was rabbit anti-goat (1:5000) for 1 hour at room temperature. FIG. 18 depicts the results from the Western blot analysis. Robust CFH expression was observed in cell samples transfected with the CFH plasmid under the control of the EF1a.SV40i; EF1a; or CRALBP promoters, while lower expression was observed in the samples transfected with the CFH plasmid under the control of the AAT1 promoter. No CFH was detected in the negative control samples. The data from the Western Blot was quantified by densitometry and the ratio between the level of CFH expression and the level of GAPDH expression for each sample was calculated (FIG. 19).

### Example 3: Transfection of HEK Cells with CFH-AAV Vectors

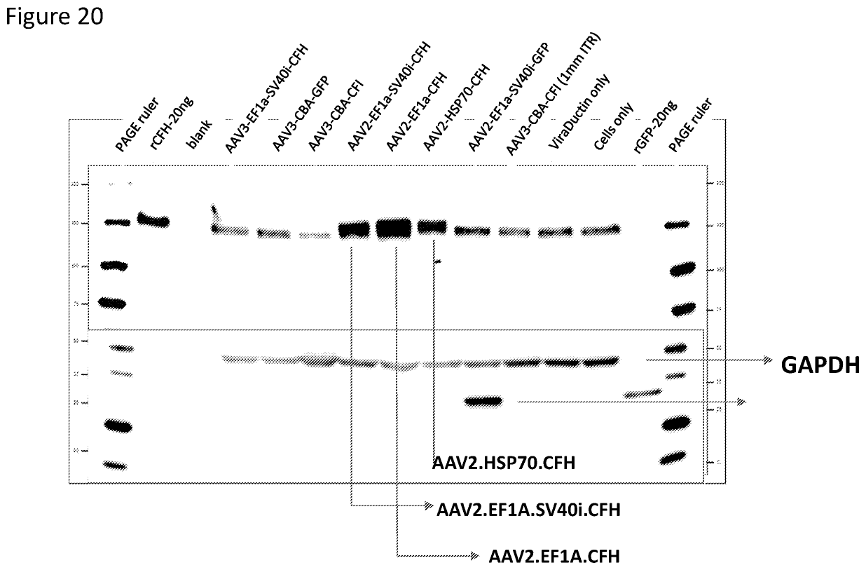

HEK-293 cells were transduced for three days with various CFH-AAV2 constructs and supernatant samples were harvested and run on a reducing PAGE gel with various controls such as recombinant CFH, recombinant GFP, untrasfected cell lysate, or cells transfected with recombinant GFP rather than CFH. Quidel goat anti-human CFH (A312) was utilized to detect CFH and the blot was incubated at a 1:1,000 dilution overnight and after washing with rabbit Anti-Goat HRP Secondary (Jackson Immunoresearch) at 1:5000 dilution for 1 hour at room temperature with rotation. The blot was separately incubated with mouse anti-eGFP antibody (Thermo Fisher, Mass. 1-952) at a 1:1,000 dilution overnight and after washing with rabbit anti-goat HRP secondary (Jackson Immunoresearch) at 1:5000 dilution for 1 hour at room temperature with rotation. The results from the Western Blot are depicted in FIG. 20. Greater expression of CFH was detected in the supernatant from cells transfected with the AAV-CFH constructs than in non-transfected or mock-transfected cells.

### Example 4: Intravitreal Treatment of Mice with AAV2-CFH Vectors

Mice were intravitreally injected with AAV2-CFH vectors under the control of the EF1a.SV40i or EF1a promoters. Eyes were collected 21 days after injection and immunohistochemistry was performed for detection of CFH protein. Eyes were embedded and section and put on slides by standard methods. Slides were washed for 3×5 minutes in 1×PBS. Sections were blocked with blocking buffer (5% BSA, 10% Donkey serum, 0.5% Triton X-100) at room temperature for 1 hour in dark humidity chamber. Samples were stained with CFH antibody (Novus cat. AF4779-SP) at a concentration of 1:20 overnight at 4° C. in dark humidity chamber. Antibody solution was prepared in blocking buffer. Slides were then washed for 3×5 minutes in 1×PBS. Samples were stained with donkey anti-goat secondary antibody (Thermofisher cat. A11056) at a concentration of 1:1000 at room temperature for 1 hour in a dark humidity chamber. The antibody solution was prepared in blocking buffer. Slides were washed for 3×5 minutes in 1×PBS. Samples were mounted with Hoechst solution and sealed and imaged. CFH was detected using the 555 (Texas Red) channel. Modest CFH protein expression was seen in the ganglion cell layer and weak expression of CFH protein was seen in the inner nuclear layer.

### Example 5: Treatment of Patients with AMD with AAV Vectors

This study will evaluate the efficacy of the vectors of Example 1 for treating patients with AMD. Patients with AMD will be treated with any of the CFH AAV2 vectors, or a control. The vectors will be administered at varying doses between 2.5×10⁸ vg to 1.4×10¹¹ vg/per eye in about 100 μl. The vectors will be administered by intravitreal injection in a solution of PBS with additional NaCl and pluronic. Patients will be monitored for improvements in AMD symptoms.

It is expected that the CFH and/or FHL1 AAV2 vector treatments will improve the AMD symptoms.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

SEQUENCE LISTING
SEQ ID NO: 1-Codon Optimized Human Factor H Like 1 (FHL1)
GCGGCCGCCACCATGAGACTTCTAGCAAAGATTATTTGCCTTATGTTATGGGCTA
TTTGTGTAGCAGAAGATTGCAATGAACTTCCTCCAAGAAGAAATACAGAAATTCT
GACAGGTTCCTGGTCTGACCAAACATATCCAGAAGGCACCCAGGCTATCTATAA
ATGCCGCCCTGGATATAGATCTCTTGGAAATGTAATAATGGTATGCAGGAAGGG
AGAATGGGTTGCTCTTAATCCATTAAGGAAATGTCAGAAAAGGCCCTGTGGACA
TCCTGGAGATACTCCTTTTGGTACTTTTACCCTTACAGGAGGAAATGTGTTTGAAT
ATGGTGTAAAAGCTGTGTATACATGTAATGAGGGGTATCAATTGCTAGGTGAGAT
TAATTACCGTGAATGTGACACAGATGGATGGACCAATGATATTCCTATATGTGAA
GTTGTGAAGTGTTTACCAGTGACAGCACCAGAGAATGGAAAAATTGTCAGTAGT
GCAATGGAACCAGATCGGGAATACCATTTTGGACAAGCAGTACGGTTTGTATGT
AACTCAGGCTACAAGATTGAAGGAGATGAAGAAATGCATTGTTCAGACGATGGT
TTTTGGAGTAAAGAGAAACCAAAGTGTGTGGAAATTTCATGCAAATCCCCAGAT
GTTATAAATGGATCTCCTATATCTCAGAAGATTATTTATAAGGAGAATGAACGAT
TTCAATATAAATGTAACATGGGTTATGAATACAGTGAAAGAGGAGATGCTGTAT
GCACTGAATCTGGATGGCGTCCGTTGCCTTCATGTGAAGAAAAATCATGTGATAA
TCCTTATATTCCAAATGGTGACTACTCACCTTTAAGGATTAAACACAGAACTGGA
GATGAAATCACGTACCAGTGTAGAAATGGTTTTTATCCTGCAACCCGGGGAAATA
CAGCAAAATGCACAAGTACTGGCTGGATACCTGCTCCGAGATGTACCTTGAAAC
CTTGTGATTATCCAGACATTAAACATGGAGGTCTATATCATGAGAATATGCGTAG
ACCATACTTTCCAGTAGCTGTAGGAAAATATTACTCCTATTACTGTGATGAACAT
TTTGAGACTCCGTCAGGAAGTTACTGGGATCACATTCATTGCACACAAGATGGAT
GGTCGCCAGCAGTACCATGCCTCAGAAAATGTTATTTTCCTTATTTGGAAAATGG
ATATAATCAAAATTATGGAAGAAAGTTTGTACAGGGTAAATCTATAGACGTTGCC
TGCCATCCTGGCTACGCTCTTCCAAAAGCGCAGACCACAGTTACATGTATGGAGA
ATGGCTGGTCTCCTACTCCCAGATGCATCCGTGTCAGCTTTACCCTCTAATAAGCT
TGGATCCAGATCT,
SEQ ID NO: 2-Codon Optimized Human Factor H Like 1 (FHL1)
GCGGCCGCCACCATGAGACTGCTGGCTAAAATTATCTGCCTGATGCTGTGGGCTA
TCTGCGTCGCTGAGGATTGTAACGAGCTGCCCCCCCGGAGAAATACAGAGATCCT
GACCGGCTCTTGGAGCGACCAGACATATCCCGAGGGCACCCAGGCCATCTACAA
GTGCAGGCCTGGCTATCGCTCTCTGGGCAACGTGATCATGGTGTGCAGGAAGGG
AGAGTGGGTGGCCCTGAATCCCCTGAGGAAGTGCCAGAAGCGCCCTTGTGGACA
CCCAGGCGACACACCCTTCGGCACCTTTACACTGACCGGCGGCAACGTGTTCGAG
TACGGCGTGAAGGCCGTGTATACCTGCAACGAGGGCTACCAGCTGCTGGGCGAG
ATCAATTACAGAGAGTGTGACACAGATGGCTGGACCAACGATATCCCTATCTGC
GAGGTGGTGAAGTGTCTGCCTGTGACCGCCCCAGAGAATGGCAAGATCGTGAGC
TCCGCCATGGAGCCAGACAGGGAGTATCACTTCGGCCAGGCCGTGCGCTTCGTGT
GCAACTCCGGCTACAAGATCGAGGGCGATGAGGAGATGCACTGTAGCGACGATG
GCTTCTGGTCCAAGGAGAAGCCCAAGTGCGTGGAGATCAGCTGTAAGTCCCCTG
ACGTGATCAATGGCTCTCCAATCAGCCAGAAGATCATCTATAAGGAGAACGAGA
GGTTTCAGTACAAGTGCAATATGGGCTACGAGTATTCTGAGAGGGGCGATGCCG
TGTGCACAGAGAGCGGATGGCGGCCCCTGCCTTCCTGCGAGGAGAAGTCTTGTG
ACAACCCTTATATCCCAAATGGCGATTACAGCCCACTGCGGATCAAGCACAGAA
CAGGCGATGAGATCACCTATCAGTGCCGGAACGGCTTTTACCCCGCCACAAGAG
GCAATACCGCCAAGTGTACATCCACCGGATGGATCCCAGCACCAAGATGCACCC
TGAAGCCCTGTGACTATCCTGATATCAAGCACGGCGGCCTGTATCACGAGAACAT
GAGACGGCCCTACTTCCCTGTGGCCGTGGGCAAGTACTATTCCTACTATTGCGAC
GAGCACTTTGAGACACCCTCCGGCTCTTACTGGGACCACATCCACTGTACCCAGG
ATGGATGGAGCCCCGCAGTGCCATGCCTGAGGAAGTGTTACTTCCCTTATCTGGA
GAATGGCTACAACCAGAATTATGGCCGCAAGTTTGTGCAGGGCAAGAGCATCGA
TGTGGCATGCCACCCAGGATACGCACTGCCAAAGGCACAGACCACAGTGACCTG
TATGGAAAACGGCTGGTCCCCTACCCCTCGCTGTATCAGAGTGTCATTCACCCTG
TAATAAGCTTGGATCCAGATCT
SEQ ID NO: 3-Non-Codon Optimized Human Factor H Like 1 (FHL1)
GCGGCCGCCACCATGAGACTTCTAGCAAAGATTATTTGCCTTATGTTATGGGCTA
TTTGTGTAGCAGAAGATTGCAATGAACTTCCTCCAAGAAGAAATACAGAAATTCT
GACAGGTTCCTGGTCTGACCAAACATATCCAGAAGGCACCCAGGCTATCTATAA
ATGCCGCCCTGGATATCGAAGTCTTGGAAATGTAATAATGGTATGCAGGAAGGG
AGAATGGGTTGCTCTTAATCCATTAAGGAAATGTCAGAAAAGGCCCTGTGGACA
TCCTGGAGATACTCCTTTTGGTACTTTTACCCTTACAGGAGGAAATGTGTTTGAAT
ATGGTGTAAAAGCTGTGTATACATGTAATGAGGGGTATCAATTGCTAGGTGAGAT
TAATTACCGTGAATGTGACACAGATGGATGGACCAATGATATTCCTATATGTGAA
GTTGTGAAGTGTTTACCAGTGACAGCACCAGAGAATGGAAAAATTGTCAGTAGT
GCAATGGAACCAGATCGGGAATACCATTTTGGACAAGCAGTACGGTTTGTATGT
AACTCAGGCTACAAGATTGAAGGAGATGAAGAAATGCATTGTTCAGACGATGGT
TTTTGGAGTAAAGAGAAACCAAAGTGTGTGGAAATTTCATGCAAATCCCCAGAT
GTTATAAATGGATCTCCTATATCTCAGAAGATTATTTATAAGGAGAATGAACGAT
TTCAATATAAATGTAACATGGGTTATGAATACAGTGAAAGAGGAGATGCTGTAT
GCACTGAATCTGGATGGCGTCCGTTGCCTTCATGTGAAGAAAAATCATGTGATAA
TCCTTATATTCCAAATGGTGACTACTCACCTTTAAGGATTAAACACAGAACTGGA
GATGAAATCACGTACCAGTGTAGAAATGGTTTTTATCCTGCAACCCGGGGAAATA
CAGCAAAATGCACAAGTACTGGCTGGATACCTGCTCCGAGATGTACCTTGAAAC
CTTGTGATTATCCAGACATTAAACATGGAGGTCTATATCATGAGAATATGCGTAG
ACCATACTTTCCAGTAGCTGTAGGAAAATATTACTCCTATTACTGTGATGAACAT
TTTGAGACTCCGTCAGGAAGTTACTGGGATCACATTCATTGCACACAAGATGGAT
GGTCGCCAGCAGTACCATGCCTCAGAAAATGTTATTTTCCTTATTTGGAAAATGG
ATATAATCAAAATTATGGAAGAAAGTTTGTACAGGGTAAATCTATAGACGTTGCC
TGCCATCCTGGCTACGCTCTTCCAAAAGCGCAGACCACAGTTACATGTATGGAGA
ATGGCTGGTCTCCTACTCCCAGATGCATCCGTGTCAGCTTTACCCTCTAATAAGCT
TGGATCCAGATCT
SEQ ID NO: 4: SFTL Sequence
SFTL
SEQ ID NO: 5-CFH Nucleotide Sequence
ATGAGACTTCTAGCAAAGATTATTTGCCTTATGTTATGGGCTATTTGTGTAGCAG
AAGATTGCAATGAACTTCCTCCAAGAAGAAATACAGAAATTCTGACAGGTTCCT
GGTCTGACCAAACATATCCAGAAGGCACCCAGGCTATCTATAAATGCCGCCCTG
GATATAGATCTCTTGGAAATGTAATAATGGTATGCAGGAAGGGAGAATGGGTTG
CTCTTAATCCATTAAGGAAATGTCAGAAAAGGCCCTGTGGACATCCTGGAGATAC
TCCTTTTGGTACTTTTACCCTTACAGGAGGAAATGTGTTTGAATATGGTGTAAAA
GCTGTGTATACATGTAATGAGGGGTATCAATTGCTAGGTGAGATTAATTACCGTG
AATGTGACACAGATGGATGGACCAATGATATTCCTATATGTGAAGTTGTGAAGTG
TTTACCAGTGACAGCACCAGAGAATGGAAAAATTGTCAGTAGTGCAATGGAACC
AGATCGGGAATACCATTTTGGACAAGCAGTACGGTTTGTATGTAACTCAGGCTAC
AAGATTGAAGGAGATGAAGAAATGCATTGTTCAGACGATGGTTTTTGGAGTAAA
GAGAAACCAAAGTGTGTGGAAATTTCATGCAAATCCCCAGATGTTATAAATGGA
TCTCCTATATCTCAGAAGATTATTTATAAGGAGAATGAACGATTTCAATATAAAT
GTAACATGGGTTATGAATACAGTGAAAGAGGAGATGCTGTATGCACTGAATCTG
GATGGCGTCCGTTGCCTTCATGTGAAGAAAAATCATGTGATAATCCTTATATTCC
AAATGGTGACTACTCACCTTTAAGGATTAAACACAGAACTGGAGATGAAATCAC
GTACCAGTGTAGAAATGGTTTTTATCCTGCAACCCGGGGAAATACAGCAAAATG
CACAAGTACTGGCTGGATACCTGCTCCGAGATGTACCTTGAAACCTTGTGATTAT
CCAGACATTAAACATGGAGGTCTATATCATGAGAATATGCGTAGACCATACTTTC
CAGTAGCTGTAGGAAAATATTACTCCTATTACTGTGATGAACATTTTGAGACTCC
GTCAGGAAGTTACTGGGATCACATTCATTGCACACAAGATGGATGGTCGCCAGC
AGTACCATGCCTCAGAAAATGTTATTTTCCTTATTTGGAAAATGGATATAATCAA
AATTATGGAAGAAAGTTTGTACAGGGTAAATCTATAGACGTTGCCTGCCATCCTG
GCTACGCTCTTCCAAAAGCGCAGACCACAGTTACATGTATGGAGAATGGCTGGTC
TCCTACTCCCAGATGCATCCGTGTCAAAACATGTTCCAAATCAAGTATAGATATT
GAGAATGGGTTTATTTCTGAATCTCAGTATACATATGCCTTAAAAGAAAAAGCGA
AATATCAATGCAAACTAGGATATGTAACAGCAGATGGTGAAACATCAGGATCAA
TTACATGTGGGAAAGATGGATGGTCAGCTCAACCCACGTGCATTAAATCTTGTGA
TATCCCAGTATTTATGAATGCCAGAACTAAAAATGACTTCACATGGTTTAAGCTG
AATGACACATTGGACTATGAATGCCATGATGGTTATGAAAGCAATACTGGAAGC
ACCACTGGTTCCATAGTGTGTGGTTACAATGGTTGGTCTGATTTACCCATATGTTA
TGAAAGAGAATGCGAACTTCCTAAAATAGATGTACACTTAGTTCCTGATCGCAAG
AAAGACCAGTATAAAGTTGGAGAGGTGTTGAAATTCTCCTGCAAACCAGGATTT
ACAATAGTTGGACCTAATTCCGTTCAGTGCTACCACTTTGGATTGTCTCCTGACCT
CCCAATATGTAAAGAGCAAGTACAATCATGTGGTCCACCTCCTGAACTCCTCAAT
GGGAATGTTAAGGAAAAAACGAAAGAAGAATATGGACACAGTGAAGTGGTGGA
ATATTATTGCAATCCTAGATTTCTAATGAAGGGACCTAATAAAATTCAATGTGTT
GATGGAGAGTGGACAACTTTACCAGTGTGTATTGTGGAGGAGAGTACCTGTGGA
GATATACCTGAACTTGAACATGGCTGGGCCCAGCTTTCTTCCCCTCCTTATTACTA
TGGAGATTCAGTGGAATTCAATTGCTCAGAATCATTTACAATGATTGGACACAGA
TCAATTACGTGTATTCATGGAGTATGGACCCAACTTCCCCAGTGTGTGGCAATAG
ATAAACTTAAGAAGTGCAAATCATCAAATTTAATTATACTTGAGGAACATTTAAA
AAACAAGAAGGAATTCGATCATAATTCTAACATAAGGTACAGATGTAGAGGAAA
AGAAGGATGGATACACACAGTCTGCATAAATGGAAGATGGGATCCAGAAGTGAA
CTGCTCAATGGCACAAATACAATTATGCCCACCTCCACCTCAGATTCCCAATTCT
CACAATATGACAACCACACTGAATTATCGGGATGGAGAAAAAGTATCTGTTCTTT
GCCAAGAAAATTATCTAATTCAGGAAGGAGAAGAAATTACATGCAAAGATGGAA
GATGGCAGTCAATACCACTCTGTGTTGAAAAAATTCCATGTTCACAACCACCTCA
GATAGAACACGGAACCATTAATTCATCCAGGTCTTCACAAGAAAGTTATGCACAT
GGGACTAAATTGAGTTATACTTGTGAGGGTGGTTTCAGGATATCTGAAGAAAATG
AAACAACATGCTACATGGGAAAATGGAGTTCTCCACCTCAGTGTGAAGGCCTTCC
TTGTAAATCTCCACCTGAGATTTCTCATGGTGTTGTAGCTCACATGTCAGACAGTT
ATCAGTATGGAGAAGAAGTTACGTACAAATGTTTTGAAGGTTTTGGAATTGATGG
GCCTGCAATTGCAAAATGCTTAGGAGAAAAATGGTCTCACCCTCCATCATGCATA
AAAACAGATTGTCTCAGTTTACCTAGCTTTGAAAATGCCATACCCATGGGAGAGA
AGAAGGATGTGTATAAGGCGGGTGAGCAAGTGACTTACACTTGTGCAACATATT
ACAAAATGGATGGAGCCAGTAATGTAACATGCATTAATAGCAGATGGACAGGAA
GGCCAACATGCAGAGACACCTCCTGTGTGAATCCGCCCACAGTACAAAATGCTT
ATATAGTGTCGAGACAGATGAGTAAATATCCATCTGGTGAGAGAGTACGTTATC
AATGTAGGAGCCCTTATGAAATGTTTGGGGATGAAGAAGTGATGTGTTTAAATG
GAAACTGGACGGAACCACCTCAATGCAAAGATTCTACAGGAAAATGTGGGCCCC
CTCCACCTATTGACAATGGGGACATTACTTCATTCCCGTTGTCAGTATATGCTCCA
GCTTCATCAGTTGAGTACCAATGCCAGAACTTGTATCAACTTGAGGGTAACAAGC
GAATAACATGTAGAAATGGACAATGGTCAGAACCACCAAAATGCTTACATCCGT
GTGTAATATCCCGAGAAATTATGGAAAATTATAACATAGCATTAAGGTGGACAG
CCAAACAGAAGCTTTATTCGAGAACAGGTGAATCAGTTGAATTTGTGTGTAAACG
GGGATATCGTCTTTCATCACGTTCTCACACATTGCGAACAACATGTTGGGATGGG
AAACTGGAGTATCCAACTTGTGCAAAAAGATAG
SEQ ID NO: 6-CRALBP Promoter
ACGCGTTAACTAGTACCCTGGTGGTGGTGGTGGGGGGGGGGGGGTGCTCTCTCA
GCAACCCCACCCCGGGATCTTGAGGAGAAAGAGGGCAGAGAAAAGAGGGAATG
GGACTGGCCCAGATCCCAGCCCCACAGCCGGGCTTCCACATGGCCGAGCAGGAA
CTCCAGAGCAGGAGCACACAAAGGAGGGCTTTGATGCGCCTCCAGCCAGGCCCA
GGCCTCTCCCCTCTCCCCTTTCTCTCTGGGTCTTCCTTTGCCCCACTGAGGGCCTC
CTGTGAGCCCGATTTAACGGAAACTGTGGGCGGTGAGAAGTTCCTTATGACACAC
TAATCCCAACCTGCTGACCGGACCACGCCTCCAGCGGAGGGAACCTCTAGAGCT
CCAGGACATTCAGGTACCAGGTAGCCCCAAGGAGGAGCTGCCGACCATCGAT
SEQ ID NO: 7-Rrepresentative CFH AAV vector (with CRALBP
Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTTAACTAGT
ACCCTGGTGGTGGTGGTGGGGGGGGGGGGGTGCTCTCTCAGCAACCCCACCCCG
GGATCTTGAGGAGAAAGAGGGCAGAGAAAAGAGGGAATGGGACTGGCCCAGAT
CCCAGCCCCACAGCCGGGCTTCCACATGGCCGAGCAGGAACTCCAGAGCAGGAG
CACACAAAGGAGGGCTTTGATGCGCCTCCAGCCAGGCCCAGGCCTCTCCCCTCTC
CCCTTTCTCTCTGGGTCTTCCTTTGCCCCACTGAGGGCCTCCTGTGAGCCCGATTT
AACGGAAACTGTGGGCGGTGAGAAGTTCCTTATGACACACTAATCCCAACCTGCT
GACCGGACCACGCCTCCAGCGGAGGGAACCTCTAGAGCTCCAGGACATTCAGGT
ACCAGGTAGCCCCAAGGAGGAGCTGCCGACCATCGATAGCTGCAGGCGGCCGCC
GCCACCATGAGACTTCTAGCAAAGATTATTTGCCTTATGTTATGGGCTATTTGTGT
AGCAGAAGATTGCAATGAACTTCCTCCAAGAAGAAATACAGAAATTCTGACAGG
TTCCTGGTCTGACCAAACATATCCAGAAGGCACCCAGGCTATCTATAAATGCCGC
CCTGGATATAGATCTCTTGGAAATGTAATAATGGTATGCAGGAAGGGAGAATGG
GTTGCTCTTAATCCATTAAGGAAATGTCAGAAAAGGCCCTGTGGACATCCTGGAG
ATACTCCTTTTGGTACTTTTACCCTTACAGGAGGAAATGTGTTTGAATATGGTGTA
AAAGCTGTGTATACATGTAATGAGGGGTATCAATTGCTAGGTGAGATTAATTACC
GTGAATGTGACACAGATGGATGGACCAATGATATTCCTATATGTGAAGTTGTGAA
GTGTTTACCAGTGACAGCACCAGAGAATGGAAAAATTGTCAGTAGTGCAATGGA
ACCAGATCGGGAATACCATTTTGGACAAGCAGTACGGTTTGTATGTAACTCAGGC
TACAAGATTGAAGGAGATGAAGAAATGCATTGTTCAGACGATGGTTTTTGGAGT
AAAGAGAAACCAAAGTGTGTGGAAATTTCATGCAAATCCCCAGATGTTATAAAT
GGATCTCCTATATCTCAGAAGATTATTTATAAGGAGAATGAACGATTTCAATATA
AATGTAACATGGGTTATGAATACAGTGAAAGAGGAGATGCTGTATGCACTGAAT
CTGGATGGCGTCCGTTGCCTTCATGTGAAGAAAAATCATGTGATAATCCTTATAT
TCCAAATGGTGACTACTCACCTTTAAGGATTAAACACAGAACTGGAGATGAAAT
CACGTACCAGTGTAGAAATGGTTTTTATCCTGCAACCCGGGGAAATACAGCAAA
ATGCACAAGTACTGGCTGGATACCTGCTCCGAGATGTACCTTGAAACCTTGTGAT
TATCCAGACATTAAACATGGAGGTCTATATCATGAGAATATGCGTAGACCATACT
TTCCAGTAGCTGTAGGAAAATATTACTCCTATTACTGTGATGAACATTTTGAGAC
TCCGTCAGGAAGTTACTGGGATCACATTCATTGCACACAAGATGGATGGTCGCCA
GCAGTACCATGCCTCAGAAAATGTTATTTTCCTTATTTGGAAAATGGATATAATC
AAAATTATGGAAGAAAGTTTGTACAGGGTAAATCTATAGACGTTGCCTGCCATCC
TGGCTACGCTCTTCCAAAAGCGCAGACCACAGTTACATGTATGGAGAATGGCTG
GTCTCCTACTCCCAGATGCATCCGTGTCAAAACATGTTCCAAATCAAGTATAGAT
ATTGAGAATGGGTTTATTTCTGAATCTCAGTATACATATGCCTTAAAAGAAAAAG
CGAAATATCAATGCAAACTAGGATATGTAACAGCAGATGGTGAAACATCAGGAT
CAATTACATGTGGGAAAGATGGATGGTCAGCTCAACCCACGTGCATTAAATCTTG
TGATATCCCAGTATTTATGAATGCCAGAACTAAAAATGACTTCACATGGTTTAAG
CTGAATGACACATTGGACTATGAATGCCATGATGGTTATGAAAGCAATACTGGA
AGCACCACTGGTTCCATAGTGTGTGGTTACAATGGTTGGTCTGATTTACCCATAT
GTTATGAAAGAGAATGCGAACTTCCTAAAATAGATGTACACTTAGTTCCTGATCG
CAAGAAAGACCAGTATAAAGTTGGAGAGGTGTTGAAATTCTCCTGCAAACCAGG
ATTTACAATAGTTGGACCTAATTCCGTTCAGTGCTACCACTTTGGATTGTCTCCTG
ACCTCCCAATATGTAAAGAGCAAGTACAATCATGTGGTCCACCTCCTGAACTCCT
CAATGGGAATGTTAAGGAAAAAACGAAAGAAGAATATGGACACAGTGAAGTGG
TGGAATATTATTGCAATCCTAGATTTCTAATGAAGGGACCTAATAAAATTCAATG
TGTTGATGGAGAGTGGACAACTTTACCAGTGTGTATTGTGGAGGAGAGTACCTGT
GGAGATATACCTGAACTTGAACATGGCTGGGCCCAGCTTTCTTCCCCTCCTTATT
ACTATGGAGATTCAGTGGAATTCAATTGCTCAGAATCATTTACAATGATTGGACA
CAGATCAATTACGTGTATTCATGGAGTATGGACCCAACTTCCCCAGTGTGTGGCA
ATAGATAAACTTAAGAAGTGCAAATCATCAAATTTAATTATACTTGAGGAACATT
TAAAAAACAAGAAGGAATTCGATCATAATTCTAACATAAGGTACAGATGTAGAG
GAAAAGAAGGATGGATACACACAGTCTGCATAAATGGAAGATGGGATCCAGAA
GTGAACTGCTCAATGGCACAAATACAATTATGCCCACCTCCACCTCAGATTCCCA
ATTCTCACAATATGACAACCACACTGAATTATCGGGATGGAGAAAAAGTATCTGT
TCTTTGCCAAGAAAATTATCTAATTCAGGAAGGAGAAGAAATTACATGCAAAGA
TGGAAGATGGCAGTCAATACCACTCTGTGTTGAAAAAATTCCATGTTCACAACCA
CCTCAGATAGAACACGGAACCATTAATTCATCCAGGTCTTCACAAGAAAGTTATG
CACATGGGACTAAATTGAGTTATACTTGTGAGGGTGGTTTCAGGATATCTGAAGA
AAATGAAACAACATGCTACATGGGAAAATGGAGTTCTCCACCTCAGTGTGAAGG
CCTTCCTTGTAAATCTCCACCTGAGATTTCTCATGGTGTTGTAGCTCACATGTCAG
ACAGTTATCAGTATGGAGAAGAAGTTACGTACAAATGTTTTGAAGGTTTTGGAAT
TGATGGGCCTGCAATTGCAAAATGCTTAGGAGAAAAATGGTCTCACCCTCCATCA
TGCATAAAAACAGATTGTCTCAGTTTACCTAGCTTTGAAAATGCCATACCCATGG
GAGAGAAGAAGGATGTGTATAAGGCGGGTGAGCAAGTGACTTACACTTGTGCAA
CATATTACAAAATGGATGGAGCCAGTAATGTAACATGCATTAATAGCAGATGGA
CAGGAAGGCCAACATGCAGAGACACCTCCTGTGTGAATCCGCCCACAGTACAAA
ATGCTTATATAGTGTCGAGACAGATGAGTAAATATCCATCTGGTGAGAGAGTAC
GTTATCAATGTAGGAGCCCTTATGAAATGTTTGGGGATGAAGAAGTGATGTGTTT
AAATGGAAACTGGACGGAACCACCTCAATGCAAAGATTCTACAGGAAAATGTGG
GCCCCCTCCACCTATTGACAATGGGGACATTACTTCATTCCCGTTGTCAGTATATG
CTCCAGCTTCATCAGTTGAGTACCAATGCCAGAACTTGTATCAACTTGAGGGTAA
CAAGCGAATAACATGTAGAAATGGACAATGGTCAGAACCACCAAAATGCTTACA
TCCGTGTGTAATATCCCGAGAAATTATGGAAAATTATAACATAGCATTAAGGTGG
ACAGCCAAACAGAAGCTTTATTCGAGAACAGGTGAATCAGTTGAATTTGTGTGTA
AACGGGGATATCGTCTTTCATCACGTTCTCACACATTGCGAACAACATGTTGGGA
TGGGAAACTGGAGTATCCAACTTGTGCAAAAAGATAGCTGTGCCTTCTAGTTGCC
AGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGG
AAGACAATAGCAGGCATGCTGGGGATGCGGTGGGCTCTATGGGTTAACTCGAGG
GATCCCGGACCGAGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCAGGG
GCGCCTGATGCGGTATTTTCTCCTTACGCATCTGTGCGGTATTTCACACCGCATAC
GTCAAAGCAACCATAGTACGCGCCCTGTAGCGGCGCATTAAGCGCGGCGGGTGT
GGTGGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGCTCTA
AATCGGGGGCTCCCTTTAGGGTTCCGATTTAGTGCTTTACGGCACCTCGACCCCA
AAAAACTTGATTTGGGTGATGGTTCACGTAGTGGGCCATCGCCCTGATAGACGGT
TTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTG
CCGATTTCGGCCTATTGGTTAAAAAATGAGCTGATTTAACAAAAATTTAACGCGA
ATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACG
AAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTT
TATTTTTCTAAATACATTCAAATATGTATCCGCTCATGAGACAATAACCCTGATA
AATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTT
TTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTC
TGACAACGATCGGAGGACCGAAGGAGCTAACCGCTTTTTTGCACAACATGGGGG
ATCATGTAACTCGCCTTGATCGTTGGGAACCGGAGCTGAATGAAGCCATACCAA
ACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAG
CACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGG
CCTTTTGCTCACATGT
SEQ ID NO: 8-EF1a Promoter
ACGCGTTAACTAGTGGGCAGAGCGCACATCGCCCACAGTCCCCGAGAAGTTGGG
GGGAGGGGTCGGCAATTGAACCGGTGCCTAGAGAAGGTGGCGCGGGGTAAACTG
GGAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCA
GAACACAG
SEQ ID NO: 9-Representative CFH AAV vector (EF1a promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTTAACTAGT
GGGCAGAGCGCACATCGCCCACAGTCCCCGAGAAGTTGGGGGGAGGGGTCGGCA
ATTGAACCGGTGCCTAGAGAAGGTGGCGCGGGGTAAACTGGGAAAGTGATGTCG
TGTACTGGCTCCGCCTTTTTCCCGAGGGTGGGGGAGAACCGTATATAAGTGCAGT
AGTCGCCGTGAACGTTCTTTTTCGCAACGGGTTTGCCGCCAGAACACAGATCGAT
AGCTGCAGGCGGCCGCCGCCACCATGAGACTTCTAGCAAAGATTATTTGCCTTAT
GTTATGGGCTATTTGTGTAGCAGAAGATTGCAATGAACTTCCTCCAAGAAGAAAT
ACAGAAATTCTGACAGGTTCCTGGTCTGACCAAACATATCCAGAAGGCACCCAG
GCTATCTATAAATGCCGCCCTGGATATAGATCTCTTGGAAATGTAATAATGGTAT
GCAGGAAGGGAGAATGGGTTGCTCTTAATCCATTAAGGAAATGTCAGAAAAGGC
CCTGTGGACATCCTGGAGATACTCCTTTTGGTACTTTTACCCTTACAGGAGGAAA
TGTGTTTGAATATGGTGTAAAAGCTGTGTATACATGTAATGAGGGGTATCAATTG
CTAGGTGAGATTAATTACCGTGAATGTGACACAGATGGATGGACCAATGATATTC
CTATATGTGAAGTTGTGAAGTGTTTACCAGTGACAGCACCAGAGAATGGAAAAA
TTGTCAGTAGTGCAATGGAACCAGATCGGGAATACCATTTTGGACAAGCAGTAC
GGTTTGTATGTAACTCAGGCTACAAGATTGAAGGAGATGAAGAAATGCATTGTTC
AGACGATGGTTTTTGGAGTAAAGAGAAACCAAAGTGTGTGGAAATTTCATGCAA
ATCCCCAGATGTTATAAATGGATCTCCTATATCTCAGAAGATTATTTATAAGGAG
AATGAACGATTTCAATATAAATGTAACATGGGTTATGAATACAGTGAAAGAGGA
GATGCTGTATGCACTGAATCTGGATGGCGTCCGTTGCCTTCATGTGAAGAAAAAT
CATGTGATAATCCTTATATTCCAAATGGTGACTACTCACCTTTAAGGATTAAACA
CAGAACTGGAGATGAAATCACGTACCAGTGTAGAAATGGTTTTTATCCTGCAACC
CGGGGAAATACAGCAAAATGCACAAGTACTGGCTGGATACCTGCTCCGAGATGT
ACCTTGAAACCTTGTGATTATCCAGACATTAAACATGGAGGTCTATATCATGAGA
ATATGCGTAGACCATACTTTCCAGTAGCTGTAGGAAAATATTACTCCTATTACTG
TGATGAACATTTTGAGACTCCGTCAGGAAGTTACTGGGATCACATTCATTGCACA
CAAGATGGATGGTCGCCAGCAGTACCATGCCTCAGAAAATGTTATTTTCCTTATT
TGGAAAATGGATATAATCAAAATTATGGAAGAAAGTTTGTACAGGGTAAATCTA
TAGACGTTGCCTGCCATCCTGGCTACGCTCTTCCAAAAGCGCAGACCACAGTTAC
ATGTATGGAGAATGGCTGGTCTCCTACTCCCAGATGCATCCGTGTCAAAACATGT
TCCAAATCAAGTATAGATATTGAGAATGGGTTTATTTCTGAATCTCAGTATACAT
ATGCCTTAAAAGAAAAAGCGAAATATCAATGCAAACTAGGATATGTAACAGCAG
ATGGTGAAACATCAGGATCAATTACATGTGGGAAAGATGGATGGTCAGCTCAAC
CCACGTGCATTAAATCTTGTGATATCCCAGTATTTATGAATGCCAGAACTAAAAA
TGACTTCACATGGTTTAAGCTGAATGACACATTGGACTATGAATGCCATGATGGT
TATGAAAGCAATACTGGAAGCACCACTGGTTCCATAGTGTGTGGTTACAATGGTT
GGTCTGATTTACCCATATGTTATGAAAGAGAATGCGAACTTCCTAAAATAGATGT
ACACTTAGTTCCTGATCGCAAGAAAGACCAGTATAAAGTTGGAGAGGTGTTGAA
ATTCTCCTGCAAACCAGGATTTACAATAGTTGGACCTAATTCCGTTCAGTGCTAC
CACTTTGGATTGTCTCCTGACCTCCCAATATGTAAAGAGCAAGTACAATCATGTG
GTCCACCTCCTGAACTCCTCAATGGGAATGTTAAGGAAAAAACGAAAGAAGAAT
ATGGACACAGTGAAGTGGTGGAATATTATTGCAATCCTAGATTTCTAATGAAGGG
ACCTAATAAAATTCAATGTGTTGATGGAGAGTGGACAACTTTACCAGTGTGTATT
GTGGAGGAGAGTACCTGTGGAGATATACCTGAACTTGAACATGGCTGGGCCCAG
CTTTCTTCCCCTCCTTATTACTATGGAGATTCAGTGGAATTCAATTGCTCAGAATC
ATTTACAATGATTGGACACAGATCAATTACGTGTATTCATGGAGTATGGACCCAA
CTTCCCCAGTGTGTGGCAATAGATAAACTTAAGAAGTGCAAATCATCAAATTTAA
TTATACTTGAGGAACATTTAAAAAACAAGAAGGAATTCGATCATAATTCTAACAT
AAGGTACAGATGTAGAGGAAAAGAAGGATGGATACACACAGTCTGCATAAATG
GAAGATGGGATCCAGAAGTGAACTGCTCAATGGCACAAATACAATTATGCCCAC
CTCCACCTCAGATTCCCAATTCTCACAATATGACAACCACACTGAATTATCGGGA
TGGAGAAAAAGTATCTGTTCTTTGCCAAGAAAATTATCTAATTCAGGAAGGAGA
AGAAATTACATGCAAAGATGGAAGATGGCAGTCAATACCACTCTGTGTTGAAAA
AATTCCATGTTCACAACCACCTCAGATAGAACACGGAACCATTAATTCATCCAGG
TCTTCACAAGAAAGTTATGCACATGGGACTAAATTGAGTTATACTTGTGAGGGTG
GTTTCAGGATATCTGAAGAAAATGAAACAACATGCTACATGGGAAAATGGAGTT
CTCCACCTCAGTGTGAAGGCCTTCCTTGTAAATCTCCACCTGAGATTTCTCATGGT
GTTGTAGCTCACATGTCAGACAGTTATCAGTATGGAGAAGAAGTTACGTACAAAT
GTTTTGAAGGTTTTGGAATTGATGGGCCTGCAATTGCAAAATGCTTAGGAGAAAA
ATGGTCTCACCCTCCATCATGCATAAAAACAGATTGTCTCAGTTTACCTAGCTTTG
AAAATGCCATACCCATGGGAGAGAAGAAGGATGTGTATAAGGCGGGTGAGCAA
GTGACTTACACTTGTGCAACATATTACAAAATGGATGGAGCCAGTAATGTAACAT
GCATTAATAGCAGATGGACAGGAAGGCCAACATGCAGAGACACCTCCTGTGTGA
ATCCGCCCACAGTACAAAATGCTTATATAGTGTCGAGACAGATGAGTAAATATCC
ATCTGGTGAGAGAGTACGTTATCAATGTAGGAGCCCTTATGAAATGTTTGGGGAT
GAAGAAGTGATGTGTTTAAATGGAAACTGGACGGAACCACCTCAATGCAAAGAT
TCTACAGGAAAATGTGGGCCCCCTCCACCTATTGACAATGGGGACATTACTTCAT
TCCCGTTGTCAGTATATGCTCCAGCTTCATCAGTTGAGTACCAATGCCAGAACTT
GTATCAACTTGAGGGTAACAAGCGAATAACATGTAGAAATGGACAATGGTCAGA
ACCACCAAAATGCTTACATCCGTGTGTAATATCCCGAGAAATTATGGAAAATTAT
AACATAGCATTAAGGTGGACAGCCAAACAGAAGCTTTATTCGAGAACAGGTGAA
TCAGTTGAATTTGTGTGTAAACGGGGATATCGTCTTTCATCACGTTCTCACACATT
GCGAACAACATGTTGGGATGGGAAACTGGAGTATCCAACTTGTGCAAAAAGATA
GCTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGC
TCTATGGGTTAACTCGAGGGATCCCGGACCGAGCGGCCGCAGGAACCCCTAGTG
ATGGAGTTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCATCTGTG
CGGTATTTCACACCGCATACGTCAAAGCAACCATAGTACGCGCCCTGTAGCGGCG
CATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAGTGG
GCCATCGCCCTGATAGACGGTTTTTCGCCCTTTGACGTTGGAGTCCACGTTCTTTA
ATAGTGGACTCTTGTTCCAAACTGGAACAACACTCAACCCTATCTCGGGCTATTC
TTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTTAT
GGTGCACTCTCAGTACAATCTGCTCTGATGCCGCATAGTTAAGCCAGCCCCGACA
CCCGCCAACACCCGCTGACGCGCCCTGACGGGCTTGTCTGCTCCCGGCATCCGCT
TACAGACAAGCTGTGACCGTCTCCGGGAGCTGCATGTGTCAGAGGTTTTCACCGT
CATCACCGAAACGCGCGAGACGAAAGGGCCTCGTGATACGCCTATTTTTATAGGT
TAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTAT
GAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTAT
GTGGCGCGGTATTATCCCGTATTGACGCCGGGCAAGAGCAACTCGGTCGCCGCAT
ACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGG
AGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCGTATCG
TAGTTATCTACACGACGGGGAGTCAGGCAACTATGGATGAACGAAATAGACAGA
TCGCTGAGATAGGTGCCTCACTGATTAAGCATTGGTAACTGTCAGACCAAGTTTA
CTCATATATACTTTAGATTGATTTAAAACTTCATTTTTAATTTAAAAGGATCTAGG
TGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTT
TTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACT
TCAAGAACTCTGTAGCACCGCCTACATACCTCGCTCTGCTAATCCTGTTACCAGT
GGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTTA
CGGTTCCTGGCCTTTTGCTGGCCTTTTGCTCACATGT
SEQ ID NO: 10-SV40i Intron
GTAAGTTTAGTCTTTTTGTCTTTTATTTCAGGTCCCGGATCCGGTGGTGGTGCAAA
TCAAAGAACTGCTCCTCAGTGGATGTTGCCTTTACTTCTAGGCCTGTACGGAAGT
GTTACTTCTGCTCTAAAAGCTGCGGAATTGTACC CGCGG
SEQ ID NO: 11-Representative CFH AAV vector (with EF1a promoter
and SV40i intron)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTTAACTAGT
GGGCAGAGCGCACATCGCCCACAGTCCCCGAGAAGTTGGGGGGAGGGGTCGGCA
ATTGAACCGGTGCCTAGAGAAGGTGGCGCGGGGTAAACTGGGAAAGTGATGTCG
TGTACTGGCTCCGCCTTTTTCCCGAGGGTGGGGGAGAACCGTATATAAGTGCAGT
AGTCGCCGTGAACGTTCTTTTTCGCAACGGGTTTGCCGCCAGAACACAGGTAAGT
TTAGTCTTTTTGTCTTTTATTTCAGGTCCCGGATCCGGTGGTGGTGCAAATCAAAG
AACTGCTCCTCAGTGGATGTTGCCTTTACTTCTAGGCCTGTACGGAAGTGTTACTT
CTGCTCTAAAAGCTGCGGAATTGTACCCGCGGATCGATAGCTGCAGGCGGCCGC
CGCCACCATGAGACTTCTAGCAAAGATTATTTGCCTTATGTTATGGGCTATTTGTG
TAGCAGAAGATTGCAATGAACTTCCTCCAAGAAGAAATACAGAAATTCTGACAG
GTTCCTGGTCTGACCAAACATATCCAGAAGGCACCCAGGCTATCTATAAATGCCG
CCCTGGATATAGATCTCTTGGAAATGTAATAATGGTATGCAGGAAGGGAGAATG
GGTTGCTCTTAATCCATTAAGGAAATGTCAGAAAAGGCCCTGTGGACATCCTGGA
GATACTCCTTTTGGTACTTTTACCCTTACAGGAGGAAATGTGTTTGAATATGGTGT
AAAAGCTGTGTATACATGTAATGAGGGGTATCAATTGCTAGGTGAGATTAATTAC
CGTGAATGTGACACAGATGGATGGACCAATGATATTCCTATATGTGAAGTTGTGA
AGTGTTTACCAGTGACAGCACCAGAGAATGGAAAAATTGTCAGTAGTGCAATGG
AACCAGATCGGGAATACCATTTTGGACAAGCAGTACGGTTTGTATGTAACTCAGG
CTACAAGATTGAAGGAGATGAAGAAATGCATTGTTCAGACGATGGTTTTTGGAG
TAAAGAGAAACCAAAGTGTGTGGAAATTTCATGCAAATCCCCAGATGTTATAAA
TGGATCTCCTATATCTCAGAAGATTATTTATAAGGAGAATGAACGATTTCAATAT
AAATGTAACATGGGTTATGAATACAGTGAAAGAGGAGATGCTGTATGCACTGAA
TCTGGATGGCGTCCGTTGCCTTCATGTGAAGAAAAATCATGTGATAATCCTTATA
TTCCAAATGGTGACTACTCACCTTTAAGGATTAAACACAGAACTGGAGATGAAAT
CACGTACCAGTGTAGAAATGGTTTTTATCCTGCAACCCGGGGAAATACAGCAAA
ATGCACAAGTACTGGCTGGATACCTGCTCCGAGATGTACCTTGAAACCTTGTGAT
TATCCAGACATTAAACATGGAGGTCTATATCATGAGAATATGCGTAGACCATACT
TTCCAGTAGCTGTAGGAAAATATTACTCCTATTACTGTGATGAACATTTTGAGAC
TCCGTCAGGAAGTTACTGGGATCACATTCATTGCACACAAGATGGATGGTCGCCA
GCAGTACCATGCCTCAGAAAATGTTATTTTCCTTATTTGGAAAATGGATATAATC
AAAATTATGGAAGAAAGTTTGTACAGGGTAAATCTATAGACGTTGCCTGCCATCC
TGGCTACGCTCTTCCAAAAGCGCAGACCACAGTTACATGTATGGAGAATGGCTG
GTCTCCTACTCCCAGATGCATCCGTGTCAAAACATGTTCCAAATCAAGTATAGAT
ATTGAGAATGGGTTTATTTCTGAATCTCAGTATACATATGCCTTAAAAGAAAAAG
CGAAATATCAATGCAAACTAGGATATGTAACAGCAGATGGTGAAACATCAGGAT
CAATTACATGTGGGAAAGATGGATGGTCAGCTCAACCCACGTGCATTAAATCTTG
TGATATCCCAGTATTTATGAATGCCAGAACTAAAAATGACTTCACATGGTTTAAG
CTGAATGACACATTGGACTATGAATGCCATGATGGTTATGAAAGCAATACTGGA
AGCACCACTGGTTCCATAGTGTGTGGTTACAATGGTTGGTCTGATTTACCCATAT
GTTATGAAAGAGAATGCGAACTTCCTAAAATAGATGTACACTTAGTTCCTGATCG
CAAGAAAGACCAGTATAAAGTTGGAGAGGTGTTGAAATTCTCCTGCAAACCAGG
ATTTACAATAGTTGGACCTAATTCCGTTCAGTGCTACCACTTTGGATTGTCTCCTG
ACCTCCCAATATGTAAAGAGCAAGTACAATCATGTGGTCCACCTCCTGAACTCCT
CAATGGGAATGTTAAGGAAAAAACGAAAGAAGAATATGGACACAGTGAAGTGG
TGGAATATTATTGCAATCCTAGATTTCTAATGAAGGGACCTAATAAAATTCAATG
TGTTGATGGAGAGTGGACAACTTTACCAGTGTGTATTGTGGAGGAGAGTACCTGT
GGAGATATACCTGAACTTGAACATGGCTGGGCCCAGCTTTCTTCCCCTCCTTATT
ACTATGGAGATTCAGTGGAATTCAATTGCTCAGAATCATTTACAATGATTGGACA
CAGATCAATTACGTGTATTCATGGAGTATGGACCCAACTTCCCCAGTGTGTGGCA
ATAGATAAACTTAAGAAGTGCAAATCATCAAATTTAATTATACTTGAGGAACATT
TAAAAAACAAGAAGGAATTCGATCATAATTCTAACATAAGGTACAGATGTAGAG
GAAAAGAAGGATGGATACACACAGTCTGCATAAATGGAAGATGGGATCCAGAA
GTGAACTGCTCAATGGCACAAATACAATTATGCCCACCTCCACCTCAGATTCCCA
ATTCTCACAATATGACAACCACACTGAATTATCGGGATGGAGAAAAAGTATCTGT
TCTTTGCCAAGAAAATTATCTAATTCAGGAAGGAGAAGAAATTACATGCAAAGA
TGGAAGATGGCAGTCAATACCACTCTGTGTTGAAAAAATTCCATGTTCACAACCA
CCTCAGATAGAACACGGAACCATTAATTCATCCAGGTCTTCACAAGAAAGTTATG
CACATGGGACTAAATTGAGTTATACTTGTGAGGGTGGTTTCAGGATATCTGAAGA
AAATGAAACAACATGCTACATGGGAAAATGGAGTTCTCCACCTCAGTGTGAAGG
CCTTCCTTGTAAATCTCCACCTGAGATTTCTCATGGTGTTGTAGCTCACATGTCAG
ACAGTTATCAGTATGGAGAAGAAGTTACGTACAAATGTTTTGAAGGTTTTGGAAT
TGATGGGCCTGCAATTGCAAAATGCTTAGGAGAAAAATGGTCTCACCCTCCATCA
TGCATAAAAACAGATTGTCTCAGTTTACCTAGCTTTGAAAATGCCATACCCATGG
GAGAGAAGAAGGATGTGTATAAGGCGGGTGAGCAAGTGACTTACACTTGTGCAA
CATATTACAAAATGGATGGAGCCAGTAATGTAACATGCATTAATAGCAGATGGA
CAGGAAGGCCAACATGCAGAGACACCTCCTGTGTGAATCCGCCCACAGTACAAA
ATGCTTATATAGTGTCGAGACAGATGAGTAAATATCCATCTGGTGAGAGAGTAC
GTTATCAATGTAGGAGCCCTTATGAAATGTTTGGGGATGAAGAAGTGATGTGTTT
AAATGGAAACTGGACGGAACCACCTCAATGCAAAGATTCTACAGGAAAATGTGG
GCCCCCTCCACCTATTGACAATGGGGACATTACTTCATTCCCGTTGTCAGTATATG
CTCCAGCTTCATCAGTTGAGTACCAATGCCAGAACTTGTATCAACTTGAGGGTAA
CAAGCGAATAACATGTAGAAATGGACAATGGTCAGAACCACCAAAATGCTTACA
TCCGTGTGTAATATCCCGAGAAATTATGGAAAATTATAACATAGCATTAAGGTGG
ACAGCCAAACAGAAGCTTTATTCGAGAACAGGTGAATCAGTTGAATTTGTGTGTA
AACGGGGATATCGTCTTTCATCACGTTCTCACACATTGCGAACAACATGTTGGGA
TGGGAAACTGGAGTATCCAACTTGTGCAAAAAGATAGCTGTGCCTTCTAGTTGCC
AGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGG
AAGACAATAGCAGGCATGCTGGGGATGCGGTGGGCTCTATGGGTTAACTCGAGG
GATCCCGGACCGAGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCAGGG
GCGCCTGATGCGGTATTTTCTCCTTACGCATCTGTGCGGTATTTCACACCGCATAC
GTCAAAGCAACCATAGTACGCGCCCTGTAGCGGCGCATTAAGCGCGGCGGGTGT
GGTGGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGCTCTA
AATCGGGGGCTCCCTTTAGGGTTCCGATTTAGTGCTTTACGGCACCTCGACCCCA
AAAAACTTGATTTGGGTGATGGTTCACGTAGTGGGCCATCGCCCTGATAGACGGT
TTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTG
CCGATTTCGGCCTATTGGTTAAAAAATGAGCTGATTTAACAAAAATTTAACGCGA
ATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACG
AAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTT
TATTTTTCTAAATACATTCAAATATGTATCCGCTCATGAGACAATAACCCTGATA
AATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTT
TTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTC
TGACAACGATCGGAGGACCGAAGGAGCTAACCGCTTTTTTGCACAACATGGGGG
ATCATGTAACTCGCCTTGATCGTTGGGAACCGGAGCTGAATGAAGCCATACCAA
ACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAG
CACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGG
CCTTTTGCTCACATGT
SEQ ID NO: 12-HSP7 Promoter
ACTAGTCCTGCAGGGCCGCCCACTCCCCCTTCCTCTCAGGGTCCCTGTCCCCTCCA
GTGAATCCCAGAAGACTCTGGAGAGTTCTGAGCAGGGGGCGGCACTCTGGCCTC
TGATTGGTCCAAGGAAGGCTGGGGGGCAGGACGGGAGGCGAAAACCCTGGAAT
ATTCCCGACCTGGCAGCCTCATCGAGCTCGGTGATTGGCTCAGAAGGGAAAAGG
CGGGTCTCCGTGACGACTTATAAAAGCCCAGGGGCAAGCGGTCCGGATAACGGC
TAGCCTGAGGAGCTGCTGCGACAGTCCACTACCTTTTTCGAGAGTGACTCCCGTT
GTCCCAAGGCTTCCCAGAGCGAACCTGTGCGGCTGCAGGCACCGGCGCGTCGAG
TTTCCGGCGTCCGGAAGGACCGAGCTCTTCTCGCGGATCCAGTGTTCCGTTTCCA
GCCCCCAATCTCAGAGCGGAGCCGACAGAGAGCAGGGAACC
SEQ ID NO: 13-Representative CFH AAV Vector (with HSP70 Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCGTTAACTAGTCCTG
CAGGGCCGCCCACTCCCCCTTCCTCTCAGGGTCCCTGTCCCCTCCAGTGAATCCC
AGAAGACTCTGGAGAGTTCTGAGCAGGGGGCGGCACTCTGGCCTCTGATTGGTC
CAAGGAAGGCTGGGGGGCAGGACGGGAGGCGAAAACCCTGGAATATTCCCGAC
CTGGCAGCCTCATCGAGCTCGGTGATTGGCTCAGAAGGGAAAAGGCGGGTCTCC
GTGACGACTTATAAAAGCCCAGGGGCAAGCGGTCCGGATAACGGCTAGCCTGAG
GAGCTGCTGCGACAGTCCACTACCTTTTTCGAGAGTGACTCCCGTTGTCCCAAGG
CTTCCCAGAGCGAACCTGTGCGGCTGCAGGCACCGGCGCGTCGAGTTTCCGGCGT
CCGGAAGGACCGAGCTCTTCTCGCGGATCCAGTGTTCCGTTTCCAGCCCCCAATC
TCAGAGCGGAGCCGACAGAGAGCAGGGAACCCTGCAGATCGATGCGGCCGCACC
ATGAGACTTCTAGCAAAGATTATTTGCCTTATGTTATGGGCTATTTGTGTAGCAG
AAGATTGCAATGAACTTCCTCCAAGAAGAAATACAGAAATTCTGACAGGTTCCT
GGTCTGACCAAACATATCCAGAAGGCACCCAGGCTATCTATAAATGCCGCCCTG
GATATAGATCTCTTGGAAATGTAATAATGGTATGCAGGAAGGGAGAATGGGTTG
CTCTTAATCCATTAAGGAAATGTCAGAAAAGGCCCTGTGGACATCCTGGAGATAC
TCCTTTTGGTACTTTTACCCTTACAGGAGGAAATGTGTTTGAATATGGTGTAAAA
GCTGTGTATACATGTAATGAGGGGTATCAATTGCTAGGTGAGATTAATTACCGTG
AATGTGACACAGATGGATGGACCAATGATATTCCTATATGTGAAGTTGTGAAGTG
TTTACCAGTGACAGCACCAGAGAATGGAAAAATTGTCAGTAGTGCAATGGAACC
AGATCGGGAATACCATTTTGGACAAGCAGTACGGTTTGTATGTAACTCAGGCTAC
AAGATTGAAGGAGATGAAGAAATGCATTGTTCAGACGATGGTTTTTGGAGTAAA
GAGAAACCAAAGTGTGTGGAAATTTCATGCAAATCCCCAGATGTTATAAATGGA
TCTCCTATATCTCAGAAGATTATTTATAAGGAGAATGAACGATTTCAATATAAAT
GTAACATGGGTTATGAATACAGTGAAAGAGGAGATGCTGTATGCACTGAATCTG
GATGGCGTCCGTTGCCTTCATGTGAAGAAAAATCATGTGATAATCCTTATATTCC
AAATGGTGACTACTCACCTTTAAGGATTAAACACAGAACTGGAGATGAAATCAC
GTACCAGTGTAGAAATGGTTTTTATCCTGCAACCCGGGGAAATACAGCAAAATG
CACAAGTACTGGCTGGATACCTGCTCCGAGATGTACCTTGAAACCTTGTGATTAT
CCAGACATTAAACATGGAGGTCTATATCATGAGAATATGCGTAGACCATACTTTC
CAGTAGCTGTAGGAAAATATTACTCCTATTACTGTGATGAACATTTTGAGACTCC
GTCAGGAAGTTACTGGGATCACATTCATTGCACACAAGATGGATGGTCGCCAGC
AGTACCATGCCTCAGAAAATGTTATTTTCCTTATTTGGAAAATGGATATAATCAA
AATTATGGAAGAAAGTTTGTACAGGGTAAATCTATAGACGTTGCCTGCCATCCTG
GCTACGCTCTTCCAAAAGCGCAGACCACAGTTACATGTATGGAGAATGGCTGGTC
TCCTACTCCCAGATGCATCCGTGTCAAAACATGTTCCAAATCAAGTATAGATATT
GAGAATGGGTTTATTTCTGAATCTCAGTATACATATGCCTTAAAAGAAAAAGCGA
AATATCAATGCAAACTAGGATATGTAACAGCAGATGGTGAAACATCAGGATCAA
TTACATGTGGGAAAGATGGATGGTCAGCTCAACCCACGTGCATTAAATCTTGTGA
TATCCCAGTATTTATGAATGCCAGAACTAAAAATGACTTCACATGGTTTAAGCTG
AATGACACATTGGACTATGAATGCCATGATGGTTATGAAAGCAATACTGGAAGC
ACCACTGGTTCCATAGTGTGTGGTTACAATGGTTGGTCTGATTTACCCATATGTTA
TGAAAGAGAATGCGAACTTCCTAAAATAGATGTACACTTAGTTCCTGATCGCAAG
AAAGACCAGTATAAAGTTGGAGAGGTGTTGAAATTCTCCTGCAAACCAGGATTT
ACAATAGTTGGACCTAATTCCGTTCAGTGCTACCACTTTGGATTGTCTCCTGACCT
CCCAATATGTAAAGAGCAAGTACAATCATGTGGTCCACCTCCTGAACTCCTCAAT
GGGAATGTTAAGGAAAAAACGAAAGAAGAATATGGACACAGTGAAGTGGTGGA
ATATTATTGCAATCCTAGATTTCTAATGAAGGGACCTAATAAAATTCAATGTGTT
GATGGAGAGTGGACAACTTTACCAGTGTGTATTGTGGAGGAGAGTACCTGTGGA
GATATACCTGAACTTGAACATGGCTGGGCCCAGCTTTCTTCCCCTCCTTATTACTA
TGGAGATTCAGTGGAATTCAATTGCTCAGAATCATTTACAATGATTGGACACAGA
TCAATTACGTGTATTCATGGAGTATGGACCCAACTTCCCCAGTGTGTGGCAATAG
ATAAACTTAAGAAGTGCAAATCATCAAATTTAATTATACTTGAGGAACATTTAAA
AAACAAGAAGGAATTCGATCATAATTCTAACATAAGGTACAGATGTAGAGGAAA
AGAAGGATGGATACACACAGTCTGCATAAATGGAAGATGGGATCCAGAAGTGAA
CTGCTCAATGGCACAAATACAATTATGCCCACCTCCACCTCAGATTCCCAATTCT
CACAATATGACAACCACACTGAATTATCGGGATGGAGAAAAAGTATCTGTTCTTT
GCCAAGAAAATTATCTAATTCAGGAAGGAGAAGAAATTACATGCAAAGATGGAA
GATGGCAGTCAATACCACTCTGTGTTGAAAAAATTCCATGTTCACAACCACCTCA
GATAGAACACGGAACCATTAATTCATCCAGGTCTTCACAAGAAAGTTATGCACAT
GGGACTAAATTGAGTTATACTTGTGAGGGTGGTTTCAGGATATCTGAAGAAAATG
AAACAACATGCTACATGGGAAAATGGAGTTCTCCACCTCAGTGTGAAGGCCTTCC
TTGTAAATCTCCACCTGAGATTTCTCATGGTGTTGTAGCTCACATGTCAGACAGTT
ATCAGTATGGAGAAGAAGTTACGTACAAATGTTTTGAAGGTTTTGGAATTGATGG
GCCTGCAATTGCAAAATGCTTAGGAGAAAAATGGTCTCACCCTCCATCATGCATA
AAAACAGATTGTCTCAGTTTACCTAGCTTTGAAAATGCCATACCCATGGGAGAGA
AGAAGGATGTGTATAAGGCGGGTGAGCAAGTGACTTACACTTGTGCAACATATT
ACAAAATGGATGGAGCCAGTAATGTAACATGCATTAATAGCAGATGGACAGGAA
GGCCAACATGCAGAGACACCTCCTGTGTGAATCCGCCCACAGTACAAAATGCTT
ATATAGTGTCGAGACAGATGAGTAAATATCCATCTGGTGAGAGAGTACGTTATC
AATGTAGGAGCCCTTATGAAATGTTTGGGGATGAAGAAGTGATGTGTTTAAATG
GAAACTGGACGGAACCACCTCAATGCAAAGATTCTACAGGAAAATGTGGGCCCC
CTCCACCTATTGACAATGGGGACATTACTTCATTCCCGTTGTCAGTATATGCTCCA
GCTTCATCAGTTGAGTACCAATGCCAGAACTTGTATCAACTTGAGGGTAACAAGC
GAATAACATGTAGAAATGGACAATGGTCAGAACCACCAAAATGCTTACATCCGT
GTGTAATATCCCGAGAAATTATGGAAAATTATAACATAGCATTAAGGTGGACAG
CCAAACAGAAGCTTTATTCGAGAACAGGTGAATCAGTTGAATTTGTGTGTAAACG
GGGATATCGTCTTTCATCACGTTCTCACACATTGCGAACAACATGTTGGGATGGG
AAACTGGAGTATCCAACTTGTGCAAAAAGATAGCTGTGCCTTCTAGTTGCCAGCC
ATCTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGA
CAATAGCAGGCATGCTGGGGATGCGGTGGGCTCTATGGGTTAACTCGAGGGATC
CCGGACCGAGCGGCCGCAGGAACCCCTAGTGATGGAGTTGGCCACTCCCTCTCTG
CGCGCTCGCTCGCTCACTGAGGCCGGGCGACCAAAGGTCGCCCGACGCCCGGGC
TTTGCCCGGGCGGCCTCAGTGAGCGAGCGAGCGCGCAGCTGCCTGCAGGGGCGC
CTGATGCGGTATTTTCTCCTTACGCATCTGTGCGGTATTTCACACCGCATACGTCA
AAGCAACCATAGTACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCCCAAAAA
ACTTGATTTGGGTGATGGTTCACGTAGTGGGCCATCGCCCTGATAGACGGTTTTT
CGCCCTTTGACGTTGGAGTCCACGTTCTTTAATAGTGGACTCTTGTTCCAAACTGG
AACAACACTCAACCCTATCTCGGGCTATTCTTTTGATTTATAAGGGATTTTGCCGA
TTTCGGCCTATTGGTTAAAAAATGAGCTGATTTAACAAAAATTTAACGCGAATTT
TAACAAAATATTAACGTTTACAATTTTATGGTGCACTCTCAGTACAATCTGCTCTG
ATGCCGCATAGTTAAGCCAGCCCCGACACCCGCCAACACCCGCTGACGCGCCCT
GACGGGCTTGTCTGCTCCCGGCATCCGCTTACAGACAAGCTGTGACCGTCTCCGG
GAGCTGCATGTGTCAGAGGTTTTCACCGTCATCACCGAAACGCGCGAGACGAAA
GGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATT
TTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAAC
TGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGAC
GCCGGGCAAGAGCAACTCGGTCGCCGCATACACTATTCTCAGAATGACTTGGTTG
AGTACTCACCAGTCACAGAAAAGCATCTTACGGATGGCATGACAGTAAGAGAAT
TATGCAGTGCTGCCATAACCATGAGTGATAACACTGCGGCCAACTTACTTCTGAC
AACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGA
CGAGCGTGACACCACGATGCCTGTAGCAATGGCAACAACGTTGCGCAAACTATT
AACTGGCGAACTACTTACTCTAGCTTCCCGGCAACAATTAATAGACTGGATGGAG
GCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGAT
TAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACC
AACTCTTTTTCCGAAGGTAACTGGCTTCAGCAGAGCGCAGATACCAAATACTGTC
CTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTT
TTGCTCACATGT
SEQ ID NO: 14-sCBA Promoter
ACTAGTCCCCACGTTCTGCTTCACTCTCCCCATCTCCCCCCCCTCCCCACCCCCAA
TTTTGTATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGCGAG
GCGGAGAGGTGCGGCGGCAGCCAATCAGAGCGGCGCGCTCCGAAAGTTTCCTTT
TATGGCGAGGCGGCGGCGGCGGCGGCCCTATAAAAAGCGAAGCGCGCGGCGGG
SEQ ID NO: 15-Representative CFH AAV Vector (with sCBA Promoter
and SV40i Intron)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTTAACTAGT
CCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGG
GCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGGGGTAAG
TTTAGTCTTTTTGTCTTTTATTTCAGGTCCCGGATCCGGTGGTGGTGCAAATCAAA
GAACTGCTCCTCAGTGGATGTTGCCTTTACTTCTAGGCCTGTACGGAAGTGTTACT
TCTGCTCTAAAAGCTGCGGAATTGTACCCGCGGATCGATAGCTGCAGGCGGCCGC
CGCCACCATGAGACTTCTAGCAAAGATTATTTGCCTTATGTTATGGGCTATTTGTG
TAGCAGAAGATTGCAATGAACTTCCTCCAAGAAGAAATACAGAAATTCTGACAG
GTTCCTGGTCTGACCAAACATATCCAGAAGGCACCCAGGCTATCTATAAATGCCG
CCCTGGATATAGATCTCTTGGAAATGTAATAATGGTATGCAGGAAGGGAGAATG
GGTTGCTCTTAATCCATTAAGGAAATGTCAGAAAAGGCCCTGTGGACATCCTGGA
GATACTCCTTTTGGTACTTTTACCCTTACAGGAGGAAATGTGTTTGAATATGGTGT
AAAAGCTGTGTATACATGTAATGAGGGGTATCAATTGCTAGGTGAGATTAATTAC
CGTGAATGTGACACAGATGGATGGACCAATGATATTCCTATATGTGAAGTTGTGA
AGTGTTTACCAGTGACAGCACCAGAGAATGGAAAAATTGTCAGTAGTGCAATGG
AACCAGATCGGGAATACCATTTTGGACAAGCAGTACGGTTTGTATGTAACTCAGG
CTACAAGATTGAAGGAGATGAAGAAATGCATTGTTCAGACGATGGTTTTTGGAG
TAAAGAGAAACCAAAGTGTGTGGAAATTTCATGCAAATCCCCAGATGTTATAAA
TGGATCTCCTATATCTCAGAAGATTATTTATAAGGAGAATGAACGATTTCAATAT
AAATGTAACATGGGTTATGAATACAGTGAAAGAGGAGATGCTGTATGCACTGAA
TCTGGATGGCGTCCGTTGCCTTCATGTGAAGAAAAATCATGTGATAATCCTTATA
TTCCAAATGGTGACTACTCACCTTTAAGGATTAAACACAGAACTGGAGATGAAAT
CACGTACCAGTGTAGAAATGGTTTTTATCCTGCAACCCGGGGAAATACAGCAAA
ATGCACAAGTACTGGCTGGATACCTGCTCCGAGATGTACCTTGAAACCTTGTGAT
TATCCAGACATTAAACATGGAGGTCTATATCATGAGAATATGCGTAGACCATACT
TTCCAGTAGCTGTAGGAAAATATTACTCCTATTACTGTGATGAACATTTTGAGAC
TCCGTCAGGAAGTTACTGGGATCACATTCATTGCACACAAGATGGATGGTCGCCA
GCAGTACCATGCCTCAGAAAATGTTATTTTCCTTATTTGGAAAATGGATATAATC
AAAATTATGGAAGAAAGTTTGTACAGGGTAAATCTATAGACGTTGCCTGCCATCC
TGGCTACGCTCTTCCAAAAGCGCAGACCACAGTTACATGTATGGAGAATGGCTG
GTCTCCTACTCCCAGATGCATCCGTGTCAAAACATGTTCCAAATCAAGTATAGAT
ATTGAGAATGGGTTTATTTCTGAATCTCAGTATACATATGCCTTAAAAGAAAAAG
CGAAATATCAATGCAAACTAGGATATGTAACAGCAGATGGTGAAACATCAGGAT
CAATTACATGTGGGAAAGATGGATGGTCAGCTCAACCCACGTGCATTAAATCTTG
TGATATCCCAGTATTTATGAATGCCAGAACTAAAAATGACTTCACATGGTTTAAG
CTGAATGACACATTGGACTATGAATGCCATGATGGTTATGAAAGCAATACTGGA
AGCACCACTGGTTCCATAGTGTGTGGTTACAATGGTTGGTCTGATTTACCCATAT
GTTATGAAAGAGAATGCGAACTTCCTAAAATAGATGTACACTTAGTTCCTGATCG
CAAGAAAGACCAGTATAAAGTTGGAGAGGTGTTGAAATTCTCCTGCAAACCAGG
ATTTACAATAGTTGGACCTAATTCCGTTCAGTGCTACCACTTTGGATTGTCTCCTG
ACCTCCCAATATGTAAAGAGCAAGTACAATCATGTGGTCCACCTCCTGAACTCCT
CAATGGGAATGTTAAGGAAAAAACGAAAGAAGAATATGGACACAGTGAAGTGG
TGGAATATTATTGCAATCCTAGATTTCTAATGAAGGGACCTAATAAAATTCAATG
TGTTGATGGAGAGTGGACAACTTTACCAGTGTGTATTGTGGAGGAGAGTACCTGT
GGAGATATACCTGAACTTGAACATGGCTGGGCCCAGCTTTCTTCCCCTCCTTATT
ACTATGGAGATTCAGTGGAATTCAATTGCTCAGAATCATTTACAATGATTGGACA
CAGATCAATTACGTGTATTCATGGAGTATGGACCCAACTTCCCCAGTGTGTGGCA
ATAGATAAACTTAAGAAGTGCAAATCATCAAATTTAATTATACTTGAGGAACATT
TAAAAAACAAGAAGGAATTCGATCATAATTCTAACATAAGGTACAGATGTAGAG
GAAAAGAAGGATGGATACACACAGTCTGCATAAATGGAAGATGGGATCCAGAA
GTGAACTGCTCAATGGCACAAATACAATTATGCCCACCTCCACCTCAGATTCCCA
ATTCTCACAATATGACAACCACACTGAATTATCGGGATGGAGAAAAAGTATCTGT
TCTTTGCCAAGAAAATTATCTAATTCAGGAAGGAGAAGAAATTACATGCAAAGA
TGGAAGATGGCAGTCAATACCACTCTGTGTTGAAAAAATTCCATGTTCACAACCA
CCTCAGATAGAACACGGAACCATTAATTCATCCAGGTCTTCACAAGAAAGTTATG
CACATGGGACTAAATTGAGTTATACTTGTGAGGGTGGTTTCAGGATATCTGAAGA
AAATGAAACAACATGCTACATGGGAAAATGGAGTTCTCCACCTCAGTGTGAAGG
CCTTCCTTGTAAATCTCCACCTGAGATTTCTCATGGTGTTGTAGCTCACATGTCAG
ACAGTTATCAGTATGGAGAAGAAGTTACGTACAAATGTTTTGAAGGTTTTGGAAT
TGATGGGCCTGCAATTGCAAAATGCTTAGGAGAAAAATGGTCTCACCCTCCATCA
TGCATAAAAACAGATTGTCTCAGTTTACCTAGCTTTGAAAATGCCATACCCATGG
GAGAGAAGAAGGATGTGTATAAGGCGGGTGAGCAAGTGACTTACACTTGTGCAA
CATATTACAAAATGGATGGAGCCAGTAATGTAACATGCATTAATAGCAGATGGA
CAGGAAGGCCAACATGCAGAGACACCTCCTGTGTGAATCCGCCCACAGTACAAA
ATGCTTATATAGTGTCGAGACAGATGAGTAAATATCCATCTGGTGAGAGAGTAC
GTTATCAATGTAGGAGCCCTTATGAAATGTTTGGGGATGAAGAAGTGATGTGTTT
AAATGGAAACTGGACGGAACCACCTCAATGCAAAGATTCTACAGGAAAATGTGG
GCCCCCTCCACCTATTGACAATGGGGACATTACTTCATTCCCGTTGTCAGTATATG
CTCCAGCTTCATCAGTTGAGTACCAATGCCAGAACTTGTATCAACTTGAGGGTAA
CAAGCGAATAACATGTAGAAATGGACAATGGTCAGAACCACCAAAATGCTTACA
TCCGTGTGTAATATCCCGAGAAATTATGGAAAATTATAACATAGCATTAAGGTGG
ACAGCCAAACAGAAGCTTTATTCGAGAACAGGTGAATCAGTTGAATTTGTGTGTA
AACGGGGATATCGTCTTTCATCACGTTCTCACACATTGCGAACAACATGTTGGGA
TGGGAAACTGGAGTATCCAACTTGTGCAAAAAGATAGCTGTGCCTTCTAGTTGCC
AGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGG
AAGACAATAGCAGGCATGCTGGGGATGCGGTGGGCTCTATGGGTTAACTCGAGG
GATCCCGGACCGAGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCAGGG
GCGCCTGATGCGGTATTTTCTCCTTACGCATCTGTGCGGTATTTCACACCGCATAC
GTCAAAGCAACCATAGTACGCGCCCTGTAGCGGCGCATTAAGCGCGGCGGGTGT
GGTGGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGCTCTA
AATCGGGGGCTCCCTTTAGGGTTCCGATTTAGTGCTTTACGGCACCTCGACCCCA
AAAAACTTGATTTGGGTGATGGTTCACGTAGTGGGCCATCGCCCTGATAGACGGT
TTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTG
CCGATTTCGGCCTATTGGTTAAAAAATGAGCTGATTTAACAAAAATTTAACGCGA
ATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACG
AAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTT
TATTTTTCTAAATACATTCAAATATGTATCCGCTCATGAGACAATAACCCTGATA
AATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTT
TTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTC
TGACAACGATCGGAGGACCGAAGGAGCTAACCGCTTTTTTGCACAACATGGGGG
ATCATGTAACTCGCCTTGATCGTTGGGAACCGGAGCTGAATGAAGCCATACCAA
ACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAG
CACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGG
CCTTTTGCTCACATGT
SEQ ID NO: 16-hAAT1 Promoter
GTTAACGGCTGCCCACTGGGCATTTCATAGGTGGCTCAGTCCTCTTCCCTCTGCA
GCTGGCCCCAGAAACCTGCCAGTTATTGGTGCCAGGTCTGTGCCAGGAGGGCGA
GGCCTGTCATTTCTAGTAATCCTCTGGGCAGTGTGACTGTACCTCTTGCGGCAACT
CAAAGGGAGAGGGTGACTTGTCCCGGGTCACAGAGCTGAAAGGGCAGGTACAAC
AGGTGACATGCCGGGCTGTCTGAGTTTATGAGGGCCCAGTCTTGTGTCTGCCGGG
CAATGAGCAAGGCTCCTTCCTGTCCAAGCTCCCCGCCCCTCCCCAGCCTACTGCC
TCCACCCGAAGTCTACTTCCTGGG
SEQ ID NO: 17-Representative CFH AAV Vector (with hAAT1 promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACG
GCTGCCCACTGGGCATTTCATAGGTGGCTCAGTCCTCTTCCCTCTGCAGCTGGCCC
CAGAAACCTGCCAGTTATTGGTGCCAGGTCTGTGCCAGGAGGGCGAGGCCTGTC
ATTTCTAGTAATCCTCTGGGCAGTGTGACTGTACCTCTTGCGGCAACTCAAAGGG
AGAGGGTGACTTGTCCCGGGTCACAGAGCTGAAAGGGCAGGTACAACAGGTGAC
ATGCCGGGCTGTCTGAGTTTATGAGGGCCCAGTCTTGTGTCTGCCGGGCAATGAG
CAAGGCTCCTTCCTGTCCAAGCTCCCCGCCCCTCCCCAGCCTACTGCCTCCACCCG
AAGTCTACTTCCTGGGATCGATAGCTGCAGGCGGCCGCCGCCACCATGAGACTTC
TAGCAAAGATTATTTGCCTTATGTTATGGGCTATTTGTGTAGCAGAAGATTGCAA
TGAACTTCCTCCAAGAAGAAATACAGAAATTCTGACAGGTTCCTGGTCTGACCAA
ACATATCCAGAAGGCACCCAGGCTATCTATAAATGCCGCCCTGGATATAGATCTC
TTGGAAATGTAATAATGGTATGCAGGAAGGGAGAATGGGTTGCTCTTAATCCATT
AAGGAAATGTCAGAAAAGGCCCTGTGGACATCCTGGAGATACTCCTTTTGGTACT
TTTACCCTTACAGGAGGAAATGTGTTTGAATATGGTGTAAAAGCTGTGTATACAT
GTAATGAGGGGTATCAATTGCTAGGTGAGATTAATTACCGTGAATGTGACACAG
ATGGATGGACCAATGATATTCCTATATGTGAAGTTGTGAAGTGTTTACCAGTGAC
AGCACCAGAGAATGGAAAAATTGTCAGTAGTGCAATGGAACCAGATCGGGAATA
CCATTTTGGACAAGCAGTACGGTTTGTATGTAACTCAGGCTACAAGATTGAAGGA
GATGAAGAAATGCATTGTTCAGACGATGGTTTTTGGAGTAAAGAGAAACCAAAG
TGTGTGGAAATTTCATGCAAATCCCCAGATGTTATAAATGGATCTCCTATATCTC
AGAAGATTATTTATAAGGAGAATGAACGATTTCAATATAAATGTAACATGGGTT
ATGAATACAGTGAAAGAGGAGATGCTGTATGCACTGAATCTGGATGGCGTCCGT
TGCCTTCATGTGAAGAAAAATCATGTGATAATCCTTATATTCCAAATGGTGACTA
CTCACCTTTAAGGATTAAACACAGAACTGGAGATGAAATCACGTACCAGTGTAG
AAATGGTTTTTATCCTGCAACCCGGGGAAATACAGCAAAATGCACAAGTACTGG
CTGGATACCTGCTCCGAGATGTACCTTGAAACCTTGTGATTATCCAGACATTAAA
CATGGAGGTCTATATCATGAGAATATGCGTAGACCATACTTTCCAGTAGCTGTAG
GAAAATATTACTCCTATTACTGTGATGAACATTTTGAGACTCCGTCAGGAAGTTA
CTGGGATCACATTCATTGCACACAAGATGGATGGTCGCCAGCAGTACCATGCCTC
AGAAAATGTTATTTTCCTTATTTGGAAAATGGATATAATCAAAATTATGGAAGAA
AGTTTGTACAGGGTAAATCTATAGACGTTGCCTGCCATCCTGGCTACGCTCTTCC
AAAAGCGCAGACCACAGTTACATGTATGGAGAATGGCTGGTCTCCTACTCCCAG
ATGCATCCGTGTCAAAACATGTTCCAAATCAAGTATAGATATTGAGAATGGGTTT
ATTTCTGAATCTCAGTATACATATGCCTTAAAAGAAAAAGCGAAATATCAATGCA
AACTAGGATATGTAACAGCAGATGGTGAAACATCAGGATCAATTACATGTGGGA
AAGATGGATGGTCAGCTCAACCCACGTGCATTAAATCTTGTGATATCCCAGTATT
TATGAATGCCAGAACTAAAAATGACTTCACATGGTTTAAGCTGAATGACACATTG
GACTATGAATGCCATGATGGTTATGAAAGCAATACTGGAAGCACCACTGGTTCC
ATAGTGTGTGGTTACAATGGTTGGTCTGATTTACCCATATGTTATGAAAGAGAAT
GCGAACTTCCTAAAATAGATGTACACTTAGTTCCTGATCGCAAGAAAGACCAGTA
TAAAGTTGGAGAGGTGTTGAAATTCTCCTGCAAACCAGGATTTACAATAGTTGGA
CCTAATTCCGTTCAGTGCTACCACTTTGGATTGTCTCCTGACCTCCCAATATGTAA
AGAGCAAGTACAATCATGTGGTCCACCTCCTGAACTCCTCAATGGGAATGTTAAG
GAAAAAACGAAAGAAGAATATGGACACAGTGAAGTGGTGGAATATTATTGCAAT
CCTAGATTTCTAATGAAGGGACCTAATAAAATTCAATGTGTTGATGGAGAGTGGA
CAACTTTACCAGTGTGTATTGTGGAGGAGAGTACCTGTGGAGATATACCTGAACT
TGAACATGGCTGGGCCCAGCTTTCTTCCCCTCCTTATTACTATGGAGATTCAGTGG
AATTCAATTGCTCAGAATCATTTACAATGATTGGACACAGATCAATTACGTGTAT
TCATGGAGTATGGACCCAACTTCCCCAGTGTGTGGCAATAGATAAACTTAAGAA
GTGCAAATCATCAAATTTAATTATACTTGAGGAACATTTAAAAAACAAGAAGGA
ATTCGATCATAATTCTAACATAAGGTACAGATGTAGAGGAAAAGAAGGATGGAT
ACACACAGTCTGCATAAATGGAAGATGGGATCCAGAAGTGAACTGCTCAATGGC
ACAAATACAATTATGCCCACCTCCACCTCAGATTCCCAATTCTCACAATATGACA
ACCACACTGAATTATCGGGATGGAGAAAAAGTATCTGTTCTTTGCCAAGAAAATT
ATCTAATTCAGGAAGGAGAAGAAATTACATGCAAAGATGGAAGATGGCAGTCAA
TACCACTCTGTGTTGAAAAAATTCCATGTTCACAACCACCTCAGATAGAACACGG
AACCATTAATTCATCCAGGTCTTCACAAGAAAGTTATGCACATGGGACTAAATTG
AGTTATACTTGTGAGGGTGGTTTCAGGATATCTGAAGAAAATGAAACAACATGCT
ACATGGGAAAATGGAGTTCTCCACCTCAGTGTGAAGGCCTTCCTTGTAAATCTCC
ACCTGAGATTTCTCATGGTGTTGTAGCTCACATGTCAGACAGTTATCAGTATGGA
GAAGAAGTTACGTACAAATGTTTTGAAGGTTTTGGAATTGATGGGCCTGCAATTG
CAAAATGCTTAGGAGAAAAATGGTCTCACCCTCCATCATGCATAAAAACAGATT
GTCTCAGTTTACCTAGCTTTGAAAATGCCATACCCATGGGAGAGAAGAAGGATGT
GTATAAGGCGGGTGAGCAAGTGACTTACACTTGTGCAACATATTACAAAATGGA
TGGAGCCAGTAATGTAACATGCATTAATAGCAGATGGACAGGAAGGCCAACATG
CAGAGACACCTCCTGTGTGAATCCGCCCACAGTACAAAATGCTTATATAGTGTCG
AGACAGATGAGTAAATATCCATCTGGTGAGAGAGTACGTTATCAATGTAGGAGC
CCTTATGAAATGTTTGGGGATGAAGAAGTGATGTGTTTAAATGGAAACTGGACG
GAACCACCTCAATGCAAAGATTCTACAGGAAAATGTGGGCCCCCTCCACCTATTG
ACAATGGGGACATTACTTCATTCCCGTTGTCAGTATATGCTCCAGCTTCATCAGTT
GAGTACCAATGCCAGAACTTGTATCAACTTGAGGGTAACAAGCGAATAACATGT
AGAAATGGACAATGGTCAGAACCACCAAAATGCTTACATCCGTGTGTAATATCC
CGAGAAATTATGGAAAATTATAACATAGCATTAAGGTGGACAGCCAAACAGAAG
CTTTATTCGAGAACAGGTGAATCAGTTGAATTTGTGTGTAAACGGGGATATCGTC
TTTCATCACGTTCTCACACATTGCGAACAACATGTTGGGATGGGAAACTGGAGTA
TCCAACTTGTGCAAAAAGATAGCTGTGCCTTCTAGTTGCCAGCCATCTGTTGTTTG
CCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGC
ATGCTGGGGATGCGGTGGGCTCTATGGGTTAACTCGAGGGATCCCGGACCGAGC
GGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTA
TTTTCTCCTTACGCATCTGTGCGGTATTTCACACCGCATACGTCAAAGCAACCATA
GTACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGATTTGG
GTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTC
AACCCTATCTCGGGCTATTCTTTTGATTTATAAGGGATTTTGCCGATTTCGGCCTA
TTGGTTAAAAAATGAGCTGATTTAACAAAAATTTAACGCGAATTTTAACAAAATA
TTAACGTTTACAATTTTATGGTGCACTCTCAGTACAATCTGCTCTGATGCCGCATA
GTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGT
GTCAGAGGTTTTCACCGTCATCACCGAAACGCGCGAGACGAAAGGGCCTCGTGA
TACGCCTATTTTTATAGGTTAATGTCATGATAATAATGGTTTCTTAGACGTCAGGT
GGCACTTTTCGGGGAAATGTGCGCGGAACCCCTATTTGTTTATTTTTCTAAATACA
TTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCA
CTTTTAAAGTTCTGCTATGTGGCGCGGTATTATCCCGTATTGACGCCGGGCAAGA
GCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCT
GCCATAACCATGAGTGATAACACTGCGGCCAACTTACTTCTGACAACGATCGGA
GGACCGAAGGAGCTAACCGCTTTTTTGCACAACATGGGGGATCATGTAACTCGCC
TTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAAC
TACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTC
TGCTAATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGA
CAGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGT
SEQ ID NO: 18-ALB Promoter
GTTAACACGCGTTAACTAGTCAGTTCCAGATGGTAAATATACACAAGGGATTTAG
TCAAACAATTTTTTGGCAAGAATATTATGAATTTTGTAATCGGTTGGCAGCCAAT
GAAATACAAAGATGAGTCTAGTTAATAATCTACAATTATTGGTTAAAGA
SEQ ID NO: 19-Representative CFH AAV Vector (with ALB Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACA
CGCGTTAACTAGTCAGTTCCAGATGGTAAATATACACAAGGGATTTAGTCAAACA
ATTTTTTGGCAAGAATATTATGAATTTTGTAATCGGTTGGCAGCCAATGAAATAC
AAAGATGAGTCTAGTTAATAATCTACAATTATTGGTTAAAGAATCGATAGCTGCA
GGCGGCCGCCGCCACCATGAGACTTCTAGCAAAGATTATTTGCCTTATGTTATGG
GCTATTTGTGTAGCAGAAGATTGCAATGAACTTCCTCCAAGAAGAAATACAGAA
ATTCTGACAGGTTCCTGGTCTGACCAAACATATCCAGAAGGCACCCAGGCTATCT
ATAAATGCCGCCCTGGATATAGATCTCTTGGAAATGTAATAATGGTATGCAGGAA
GGGAGAATGGGTTGCTCTTAATCCATTAAGGAAATGTCAGAAAAGGCCCTGTGG
ACATCCTGGAGATACTCCTTTTGGTACTTTTACCCTTACAGGAGGAAATGTGTTTG
AATATGGTGTAAAAGCTGTGTATACATGTAATGAGGGGTATCAATTGCTAGGTGA
GATTAATTACCGTGAATGTGACACAGATGGATGGACCAATGATATTCCTATATGT
GAAGTTGTGAAGTGTTTACCAGTGACAGCACCAGAGAATGGAAAAATTGTCAGT
AGTGCAATGGAACCAGATCGGGAATACCATTTTGGACAAGCAGTACGGTTTGTA
TGTAACTCAGGCTACAAGATTGAAGGAGATGAAGAAATGCATTGTTCAGACGAT
GGTTTTTGGAGTAAAGAGAAACCAAAGTGTGTGGAAATTTCATGCAAATCCCCA
GATGTTATAAATGGATCTCCTATATCTCAGAAGATTATTTATAAGGAGAATGAAC
GATTTCAATATAAATGTAACATGGGTTATGAATACAGTGAAAGAGGAGATGCTG
TATGCACTGAATCTGGATGGCGTCCGTTGCCTTCATGTGAAGAAAAATCATGTGA
TAATCCTTATATTCCAAATGGTGACTACTCACCTTTAAGGATTAAACACAGAACT
GGAGATGAAATCACGTACCAGTGTAGAAATGGTTTTTATCCTGCAACCCGGGGA
AATACAGCAAAATGCACAAGTACTGGCTGGATACCTGCTCCGAGATGTACCTTG
AAACCTTGTGATTATCCAGACATTAAACATGGAGGTCTATATCATGAGAATATGC
GTAGACCATACTTTCCAGTAGCTGTAGGAAAATATTACTCCTATTACTGTGATGA
ACATTTTGAGACTCCGTCAGGAAGTTACTGGGATCACATTCATTGCACACAAGAT
GGATGGTCGCCAGCAGTACCATGCCTCAGAAAATGTTATTTTCCTTATTTGGAAA
ATGGATATAATCAAAATTATGGAAGAAAGTTTGTACAGGGTAAATCTATAGACG
TTGCCTGCCATCCTGGCTACGCTCTTCCAAAAGCGCAGACCACAGTTACATGTAT
GGAGAATGGCTGGTCTCCTACTCCCAGATGCATCCGTGTCAAAACATGTTCCAAA
TCAAGTATAGATATTGAGAATGGGTTTATTTCTGAATCTCAGTATACATATGCCTT
AAAAGAAAAAGCGAAATATCAATGCAAACTAGGATATGTAACAGCAGATGGTG
AAACATCAGGATCAATTACATGTGGGAAAGATGGATGGTCAGCTCAACCCACGT
GCATTAAATCTTGTGATATCCCAGTATTTATGAATGCCAGAACTAAAAATGACTT
CACATGGTTTAAGCTGAATGACACATTGGACTATGAATGCCATGATGGTTATGAA
AGCAATACTGGAAGCACCACTGGTTCCATAGTGTGTGGTTACAATGGTTGGTCTG
ATTTACCCATATGTTATGAAAGAGAATGCGAACTTCCTAAAATAGATGTACACTT
AGTTCCTGATCGCAAGAAAGACCAGTATAAAGTTGGAGAGGTGTTGAAATTCTC
CTGCAAACCAGGATTTACAATAGTTGGACCTAATTCCGTTCAGTGCTACCACTTT
GGATTGTCTCCTGACCTCCCAATATGTAAAGAGCAAGTACAATCATGTGGTCCAC
CTCCTGAACTCCTCAATGGGAATGTTAAGGAAAAAACGAAAGAAGAATATGGAC
ACAGTGAAGTGGTGGAATATTATTGCAATCCTAGATTTCTAATGAAGGGACCTAA
TAAAATTCAATGTGTTGATGGAGAGTGGACAACTTTACCAGTGTGTATTGTGGAG
GAGAGTACCTGTGGAGATATACCTGAACTTGAACATGGCTGGGCCCAGCTTTCTT
CCCCTCCTTATTACTATGGAGATTCAGTGGAATTCAATTGCTCAGAATCATTTACA
ATGATTGGACACAGATCAATTACGTGTATTCATGGAGTATGGACCCAACTTCCCC
AGTGTGTGGCAATAGATAAACTTAAGAAGTGCAAATCATCAAATTTAATTATACT
TGAGGAACATTTAAAAAACAAGAAGGAATTCGATCATAATTCTAACATAAGGTA
CAGATGTAGAGGAAAAGAAGGATGGATACACACAGTCTGCATAAATGGAAGAT
GGGATCCAGAAGTGAACTGCTCAATGGCACAAATACAATTATGCCCACCTCCAC
CTCAGATTCCCAATTCTCACAATATGACAACCACACTGAATTATCGGGATGGAGA
AAAAGTATCTGTTCTTTGCCAAGAAAATTATCTAATTCAGGAAGGAGAAGAAATT
ACATGCAAAGATGGAAGATGGCAGTCAATACCACTCTGTGTTGAAAAAATTCCA
TGTTCACAACCACCTCAGATAGAACACGGAACCATTAATTCATCCAGGTCTTCAC
AAGAAAGTTATGCACATGGGACTAAATTGAGTTATACTTGTGAGGGTGGTTTCAG
GATATCTGAAGAAAATGAAACAACATGCTACATGGGAAAATGGAGTTCTCCACC
TCAGTGTGAAGGCCTTCCTTGTAAATCTCCACCTGAGATTTCTCATGGTGTTGTAG
CTCACATGTCAGACAGTTATCAGTATGGAGAAGAAGTTACGTACAAATGTTTTGA
AGGTTTTGGAATTGATGGGCCTGCAATTGCAAAATGCTTAGGAGAAAAATGGTCT
CACCCTCCATCATGCATAAAAACAGATTGTCTCAGTTTACCTAGCTTTGAAAATG
CCATACCCATGGGAGAGAAGAAGGATGTGTATAAGGCGGGTGAGCAAGTGACTT
ACACTTGTGCAACATATTACAAAATGGATGGAGCCAGTAATGTAACATGCATTA
ATAGCAGATGGACAGGAAGGCCAACATGCAGAGACACCTCCTGTGTGAATCCGC
CCACAGTACAAAATGCTTATATAGTGTCGAGACAGATGAGTAAATATCCATCTGG
TGAGAGAGTACGTTATCAATGTAGGAGCCCTTATGAAATGTTTGGGGATGAAGA
AGTGATGTGTTTAAATGGAAACTGGACGGAACCACCTCAATGCAAAGATTCTAC
AGGAAAATGTGGGCCCCCTCCACCTATTGACAATGGGGACATTACTTCATTCCCG
TTGTCAGTATATGCTCCAGCTTCATCAGTTGAGTACCAATGCCAGAACTTGTATC
AACTTGAGGGTAACAAGCGAATAACATGTAGAAATGGACAATGGTCAGAACCAC
CAAAATGCTTACATCCGTGTGTAATATCCCGAGAAATTATGGAAAATTATAACAT
AGCATTAAGGTGGACAGCCAAACAGAAGCTTTATTCGAGAACAGGTGAATCAGT
TGAATTTGTGTGTAAACGGGGATATCGTCTTTCATCACGTTCTCACACATTGCGA
ACAACATGTTGGGATGGGAAACTGGAGTATCCAACTTGTGCAAAAAGATAGCTG
TGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTA
TGGGTTAACTCGAGGGATCCCGGACCGAGCGGCCGCAGGAACCCCTAGTGATGG
AGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCATT
AAGCGCGGCGGGTGTGGTGGTTACGCGCAGCGTGACCGCTACACTTGCCAGCGC
CCTAGCGCCCGCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTAGTGCTTTAC
GGCACCTCGACCCCAAAAAACTTGATTTGGGTGATGGTTCACGTAGTGGGCCATC
GCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTTTTGAT
TTATAAGGGATTTTGCCGATTTCGGCCTATTGGTTAAAAAATGAGCTGATTTAAC
AAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCA
ACACCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTC
ATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGT
SEQ ID NO: 20-PCK1 Promoter
GTTAACAGCCCCCAGTTAGGTTAGGCATTTCCAATCTTTGCCAATAAGCCACATA
TTTGCCCAAGTTAGGGTGCATCCTTCCCATGAACTTTGACTGTGACCTTTGACTAT
GGGGTGACATCTTATAGCTGTGGTGTTTTGCCAACCAGCAGCTCTTGGTACACAA
AATGTGCTGCTAGCAGGTGCCCCGGCCAACCTTGTCCTTGACCCACCTGCCTGTT
AAGAAAAGGGTGTTGTGTTTTGCAACAGCAGTAAAATGGGTCAAGGTTTAGTCA
GTTGGAAGTTGTGTCAAAACTCACTATGGTTGGTTGAGGGCTCGAAGTCTCCCAG
CATTCATTAACAACTATCTGTTCAATGATTATCTCCCTGGGGCGTGTTGCAGTGA
GTTGGCCCAAAGCATAACTGACCCTGGCCGTGATCCAGAGACCTGCCCCCTGACG
TCAGTGGCGAGCCTCCCTGGGTGCAGCTGAGGGGCAGGGCTATTCTTTTCCACAG
T
SEQ ID NO: 21-Representative CFH AAV Vector (with PCK1 Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACA
GCCCCCAGTTAGGTTAGGCATTTCCAATCTTTGCCAATAAGCCACATATTTGCCC
AAGTTAGGGTGCATCCTTCCCATGAACTTTGACTGTGACCTTTGACTATGGGGTG
ACATCTTATAGCTGTGGTGTTTTGCCAACCAGCAGCTCTTGGTACACAAAATGTG
CTGCTAGCAGGTGCCCCGGCCAACCTTGTCCTTGACCCACCTGCCTGTTAAGAAA
AGGGTGTTGTGTTTTGCAACAGCAGTAAAATGGGTCAAGGTTTAGTCAGTTGGAA
GTTGTGTCAAAACTCACTATGGTTGGTTGAGGGCTCGAAGTCTCCCAGCATTCAT
TAACAACTATCTGTTCAATGATTATCTCCCTGGGGCGTGTTGCAGTGAGTTGGCC
CAAAGCATAACTGACCCTGGCCGTGATCCAGAGACCTGCCCCCTGACGTCAGTG
GCGAGCCTCCCTGGGTGCAGCTGAGGGGCAGGGCTATTCTTTTCCACAGTATCGA
TAGCTGCAGGCGGCCGCCGCCACCATGAGACTTCTAGCAAAGATTATTTGCCTTA
TGTTATGGGCTATTTGTGTAGCAGAAGATTGCAATGAACTTCCTCCAAGAAGAAA
TACAGAAATTCTGACAGGTTCCTGGTCTGACCAAACATATCCAGAAGGCACCCA
GGCTATCTATAAATGCCGCCCTGGATATAGATCTCTTGGAAATGTAATAATGGTA
TGCAGGAAGGGAGAATGGGTTGCTCTTAATCCATTAAGGAAATGTCAGAAAAGG
CCCTGTGGACATCCTGGAGATACTCCTTTTGGTACTTTTACCCTTACAGGAGGAA
ATGTGTTTGAATATGGTGTAAAAGCTGTGTATACATGTAATGAGGGGTATCAATT
GCTAGGTGAGATTAATTACCGTGAATGTGACACAGATGGATGGACCAATGATAT
TCCTATATGTGAAGTTGTGAAGTGTTTACCAGTGACAGCACCAGAGAATGGAAA
AATTGTCAGTAGTGCAATGGAACCAGATCGGGAATACCATTTTGGACAAGCAGT
ACGGTTTGTATGTAACTCAGGCTACAAGATTGAAGGAGATGAAGAAATGCATTG
TTCAGACGATGGTTTTTGGAGTAAAGAGAAACCAAAGTGTGTGGAAATTTCATGC
AAATCCCCAGATGTTATAAATGGATCTCCTATATCTCAGAAGATTATTTATAAGG
AGAATGAACGATTTCAATATAAATGTAACATGGGTTATGAATACAGTGAAAGAG
GAGATGCTGTATGCACTGAATCTGGATGGCGTCCGTTGCCTTCATGTGAAGAAAA
ATCATGTGATAATCCTTATATTCCAAATGGTGACTACTCACCTTTAAGGATTAAA
CACAGAACTGGAGATGAAATCACGTACCAGTGTAGAAATGGTTTTTATCCTGCAA
CCCGGGGAAATACAGCAAAATGCACAAGTACTGGCTGGATACCTGCTCCGAGAT
GTACCTTGAAACCTTGTGATTATCCAGACATTAAACATGGAGGTCTATATCATGA
GAATATGCGTAGACCATACTTTCCAGTAGCTGTAGGAAAATATTACTCCTATTAC
TGTGATGAACATTTTGAGACTCCGTCAGGAAGTTACTGGGATCACATTCATTGCA
CACAAGATGGATGGTCGCCAGCAGTACCATGCCTCAGAAAATGTTATTTTCCTTA
TTTGGAAAATGGATATAATCAAAATTATGGAAGAAAGTTTGTACAGGGTAAATC
TATAGACGTTGCCTGCCATCCTGGCTACGCTCTTCCAAAAGCGCAGACCACAGTT
ACATGTATGGAGAATGGCTGGTCTCCTACTCCCAGATGCATCCGTGTCAAAACAT
GTTCCAAATCAAGTATAGATATTGAGAATGGGTTTATTTCTGAATCTCAGTATAC
ATATGCCTTAAAAGAAAAAGCGAAATATCAATGCAAACTAGGATATGTAACAGC
AGATGGTGAAACATCAGGATCAATTACATGTGGGAAAGATGGATGGTCAGCTCA
ACCCACGTGCATTAAATCTTGTGATATCCCAGTATTTATGAATGCCAGAACTAAA
AATGACTTCACATGGTTTAAGCTGAATGACACATTGGACTATGAATGCCATGATG
GTTATGAAAGCAATACTGGAAGCACCACTGGTTCCATAGTGTGTGGTTACAATGG
TTGGTCTGATTTACCCATATGTTATGAAAGAGAATGCGAACTTCCTAAAATAGAT
GTACACTTAGTTCCTGATCGCAAGAAAGACCAGTATAAAGTTGGAGAGGTGTTG
AAATTCTCCTGCAAACCAGGATTTACAATAGTTGGACCTAATTCCGTTCAGTGCT
ACCACTTTGGATTGTCTCCTGACCTCCCAATATGTAAAGAGCAAGTACAATCATG
TGGTCCACCTCCTGAACTCCTCAATGGGAATGTTAAGGAAAAAACGAAAGAAGA
ATATGGACACAGTGAAGTGGTGGAATATTATTGCAATCCTAGATTTCTAATGAAG
GGACCTAATAAAATTCAATGTGTTGATGGAGAGTGGACAACTTTACCAGTGTGTA
TTGTGGAGGAGAGTACCTGTGGAGATATACCTGAACTTGAACATGGCTGGGCCC
AGCTTTCTTCCCCTCCTTATTACTATGGAGATTCAGTGGAATTCAATTGCTCAGAA
TCATTTACAATGATTGGACACAGATCAATTACGTGTATTCATGGAGTATGGACCC
AACTTCCCCAGTGTGTGGCAATAGATAAACTTAAGAAGTGCAAATCATCAAATTT
AATTATACTTGAGGAACATTTAAAAAACAAGAAGGAATTCGATCATAATTCTAA
CATAAGGTACAGATGTAGAGGAAAAGAAGGATGGATACACACAGTCTGCATAAA
TGGAAGATGGGATCCAGAAGTGAACTGCTCAATGGCACAAATACAATTATGCCC
ACCTCCACCTCAGATTCCCAATTCTCACAATATGACAACCACACTGAATTATCGG
GATGGAGAAAAAGTATCTGTTCTTTGCCAAGAAAATTATCTAATTCAGGAAGGA
GAAGAAATTACATGCAAAGATGGAAGATGGCAGTCAATACCACTCTGTGTTGAA
AAAATTCCATGTTCACAACCACCTCAGATAGAACACGGAACCATTAATTCATCCA
GGTCTTCACAAGAAAGTTATGCACATGGGACTAAATTGAGTTATACTTGTGAGGG
TGGTTTCAGGATATCTGAAGAAAATGAAACAACATGCTACATGGGAAAATGGAG
TTCTCCACCTCAGTGTGAAGGCCTTCCTTGTAAATCTCCACCTGAGATTTCTCATG
GTGTTGTAGCTCACATGTCAGACAGTTATCAGTATGGAGAAGAAGTTACGTACAA
ATGTTTTGAAGGTTTTGGAATTGATGGGCCTGCAATTGCAAAATGCTTAGGAGAA
AAATGGTCTCACCCTCCATCATGCATAAAAACAGATTGTCTCAGTTTACCTAGCT
TTGAAAATGCCATACCCATGGGAGAGAAGAAGGATGTGTATAAGGCGGGTGAGC
AAGTGACTTACACTTGTGCAACATATTACAAAATGGATGGAGCCAGTAATGTAA
CATGCATTAATAGCAGATGGACAGGAAGGCCAACATGCAGAGACACCTCCTGTG
TGAATCCGCCCACAGTACAAAATGCTTATATAGTGTCGAGACAGATGAGTAAAT
ATCCATCTGGTGAGAGAGTACGTTATCAATGTAGGAGCCCTTATGAAATGTTTGG
GGATGAAGAAGTGATGTGTTTAAATGGAAACTGGACGGAACCACCTCAATGCAA
AGATTCTACAGGAAAATGTGGGCCCCCTCCACCTATTGACAATGGGGACATTACT
TCATTCCCGTTGTCAGTATATGCTCCAGCTTCATCAGTTGAGTACCAATGCCAGA
ACTTGTATCAACTTGAGGGTAACAAGCGAATAACATGTAGAAATGGACAATGGT
CAGAACCACCAAAATGCTTACATCCGTGTGTAATATCCCGAGAAATTATGGAAA
ATTATAACATAGCATTAAGGTGGACAGCCAAACAGAAGCTTTATTCGAGAACAG
GTGAATCAGTTGAATTTGTGTGTAAACGGGGATATCGTCTTTCATCACGTTCTCA
CACATTGCGAACAACATGTTGGGATGGGAAACTGGAGTATCCAACTTGTGCAAA
AAGATAGCTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCG
GTGGGCTCTATGGGTTAACTCGAGGGATCCCGGACCGAGCGGCCGCAGGAACCC
CTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCG
AGCGAGCGCGCAGCTGCCTGCAGGGGCGCCTGATGCGGTATTTTCTCCTTACGCA
TCTGTGCGGTATTTCACACCGCATACGTCAAAGCAACCATAGTACGCGCCCTGTA
GCGGCGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTCCGATTT
AGTGCTTTACGGCACCTCGACCCCAAAAAACTTGATTTGGGTGATGGTTCACGTA
GTGGGCCATCGCCCTGATAGACGGTTTTTCGCCCTTTGACGTTGGAGTCCACGTT
CTTTAATAGTGGACTCTTGTTCCAAACTGGAACAACACTCAACCCTATCTCGGGC
TATTCTTTTGATTTATAAGGGATTTTGCCGATTTCGGCCTATTGGTTAAAAAATGA
GCTGATTTAACAAAAATTTAACGCGAATTTTAACAAAATATTAACGTTTACAATT
TTATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATC
CGCTTACAGACAAGCTGTGACCGTCTCCGGGAGCTGCATGTGTCAGAGGTTTTCA
CCGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGA
GTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCC
TTGAGAGTTTTCGCCCCGAAGAACGTTTTCCAATGATGAGCACTTTTAAAGTTCT
GCTATGTGGCGCGGTATTATCCCGTATTGACGCCGGGCAAGAGCAACTCGGTCGC
CGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGA
GTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGT
AGCAATGGCAACAACGTTGCGCAAACTATTAACTGGCGAACTACTTACTCTAGCT
TCCCGGCAACAATTAATAGACTGGATGGAGGCGGATAAAGTTGCAGGACCACTT
CTGCGCTCGGCCCTTCCGGCTGGCTGGTTTATTGCTGATAAATCTGGAGCCGGTG
AGCGTGGGTCTCGCGGTATCATTGCAGCACTGGGGCCAGATGGTAAGCCCTCCCG
TATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGAT
CTAGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATCC
TTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAAGGTAACTGGCT
TCAGCAGAGCGCAGATACCAAATACTGTCCTTCTAGTGTAGCCGTAGTTAGGCCA
CCACTTCAAGAACTCTGTAGCACCGCCTACATACCTCGCTCTGCTAATCCTGTTAC
CAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGT
SEQ ID NO: 22-Representative FHL AAV Vector (with EF1a Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGGG
GCAGAGCGCACATCGCCCACAGTCCCCGAGAAGTTGGGGGGAGGGGTCGG
CAATTGAACCGGTGCCTAGAGAAGGTGGCGCGGGGTAAACTGGGAAAGTG
ATGTCGTGTACTGGCTCCGCCTTTTTCCCGAGGGTGGGGGAGAACCGTAT
ATAAGTGCAGTAGTCGCCGTGAACGTTCTTTTTCGCAACGGGTTTGCCGC
CAGAACACAGACCGGTCTCGAAGGCCTGCAGGCGGCCGCCGCCACCATGA
ATGAGACTTCTAGCAAAGATTATTTGCCTTATGTTATGGGCTATTTGTGT
AGCAGAAGATTGCAATGAACTTCCTCCAAGAAGAAATACAGAAATTCTGA
CAGGTTCCTGGTCTGACCAAACATATCCAGAAGGCACCCAGGCTATCTAT
AAATGCCGCCCTGGATATAGATCTCTTGGAAATGTAATAATGGTATGCAG
GAAGGGAGAATGGGTTGCTCTTAATCCATTAAGGAAATGTCAGAAAAGGC
CCTGTGGACATCCTGGAGATACTCCTTTTGGTACTTTTACCCTTACAGGA
GGAAATGTGTTTGAATATGGTGTAAAAGCTGTGTATACATGTAATGAGGG
GTATCAATTGCTAGGTGAGATTAATTACCGTGAATGTGACACAGATGGAT
GGACCAATGATATTCCTATATGTGAAGTTGTGAAGTGTTTACCAGTGACA
GCACCAGAGAATGGAAAAATTGTCAGTAGTGCAATGGAACCAGATCGGGA
ATACCATTTTGGACAAGCAGTACGGTTTGTATGTAACTCAGGCTACAAGA
TTGAAGGAGATGAAGAAATGCATTGTTCAGACGATGGTTTTTGGAGTAAA
GAGAAACCAAAGTGTGTGGAAATTTCATGCAAATCCCCAGATGTTATAAA
TGGATCTCCTATATCTCAGAAGATTATTTATAAGGAGAATGAACGATTTC
AATATAAATGTAACATGGGTTATGAATACAGTGAAAGAGGAGATGCTGTA
TGCACTGAATCTGGATGGCGTCCGTTGCCTTCATGTGAAGAAAAATCATG
TGATAATCCTTATATTCCAAATGGTGACTACTCACCTTTAAGGATTAAAC
ACAGAACTGGAGATGAAATCACGTACCAGTGTAGAAATGGTTTTTATCCT
GCAACCCGGGGAAATACAGCAAAATGCACAAGTACTGGCTGGATACCTGC
TCCGAGATGTACCTTGAAACCTTGTGATTATCCAGACATTAAACATGGAG
GTCTATATCATGAGAATATGCGTAGACCATACTTTCCAGTAGCTGTAGGA
AAATATTACTCCTATTACTGTGATGAACATTTTGAGACTCCGTCAGGAAG
TTACTGGGATCACATTCATTGCACACAAGATGGATGGTCGCCAGCAGTAC
CATGCCTCAGAAAATGTTATTTTCCTTATTTGGAAAATGGATATAATCAA
AATCATGGAAGAAAGTTTGTACAGGGTAAATCTATAGACGTTGCCTGCCA
TCCTGGCTACGCTCTTCCAAAAGCGCAGACCACAGTTACATGTATGGAGA
ATGGCTGGTCTCCTACTCCCAGATGCATCCGTGTCAGCTTTACCCTCTGA
TAAGATATCGATACATTGATGAGTTTGGACAAACCACAACTAGAATGCAG
TGAAAAAAATGCTTTATTTGTGAAATTTGTGATGCTATTGCTTTATTTGT
AACCATTATAAGCTGCAATAAACAAGATATCGTTAACTCGAGGGATCCCA
CGTGCTGATTTTGTAGGTAACCACGTGCGGACCGAGCGGCCGCAGGAACC
CCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCC
TCAGTGAGCGAGCGAGCGCGCAGCTGCCTGCAGGGGCGCCTGATGCGGTA
TTTTCTCCTTACGCATCTGTGCGGTATTTCACACCGCATACGTCAAAGCA
ACCATAGTACGCGCCCTGTAGCGGCGCATTAAGCGCGGCGGGTGTGGTGG
TTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGC
ACCTCGACCCCAAAAAACTTGATTTGGGTGATGGTTCACGTAGTGGGCCA
TCGCCCTGATAGACGGTTTTTCGCCCTTTGACGTTGGAGTCCACGTTCTT
TAATAGTGGACTCTTGTTCCAAACTGGAACAACACTCAACCCTATCTCGG
GCTATTCTTTTGATTTATAAGGGATTTTGCCGATTTCGGCCTATTGGTTA
AAAAATGAGCTGATTTAACAAAAATTTAACGCGAATTTTAACAAAATATT
AACGTTTACAATTTTATGGTGCACTCTCAGTACAATCTGCTCTGATGCCG
CATAGTTAAGCCAGCCCCGACACCCGCCAACACCCGCTGACGCGCCCTGA
CGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGA
GACGAAAGGGCCTCGTGATACGCCTATTTTTATAGGTTAATGTCATGATA
ATAATGGTTTCTTAGACGTCAGGTGGCACTTTTCGGGGAAATGTGCGCGG
AACCCCTATTTGTTTATTTTTCTAAATACATTCAAATATGTATCCGCTCA
TGAGACAATAACCCTGATAAATGCTTCAATAATATTGAAAAAGGAAGAGT
ATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATG
CTGAAGATCAGTTGGGTGCACGAGTGGGTTACATCGAACTGGATCTCAAC
AGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACG
CCGGGCAAGAGCAACTCGGTCGCCGCATACACTATTCTCAGAATGACTTG
GTTGAGTACTCACCAGTCACAGAAAAGCATCTTACGGATGGCATGACAGT
AAGAGAATTATGCAGTGCTGCCATAACCATGAGTGATAACACTGCGGCCA
ACTTACTTCTGACAACGATCGGAGGACCGAAGGAGCTAACCGCTTTTTTG
CACAACATGGGGGATCATGTAACTCGCCTTGATCGTTGGGAACCGGAGCT
GAATGAAGCCATACCAAACGACGAGCGTGACACCACGATGCCTGTAGCAA
TGGCAACAACGTTGCGCAAACTATTAACTGGCGAACTACTTACTCTAGCT
TCCCGGCAACAATTAATAGACTGGATGGAGGCGGATAAAGTTGCAGGACC
ACTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAAC
TATGGATGAACGAAATAGACAGATCGCTGAGATAGGTGCCTCACTGATTA
AGCATTGGTAACTGTCAGACCAAGTTTACTCATATATACTTTAGATTGAT
TTAAAACTTCATTTTTAATTTAAAAGGATCTAGGTGAAGATCCTTTTTGA
TAATCTCATGACCAAAATCCCTTAACGTGAGTTTTCGTTCCACTGAGCGT
CAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCT
TCAGCAGAGCGCAGATACCAAATACTGTCCTTCTAGTGTAGCCGTAGTTA
GGCCACCACTTCAAGAACTCTGTAGCACCGCCTACATACCTCGCTCTGCT
AATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGA
ACGGGGGGTTCGTGCACACAGCCCAGCTTGGAGCGAACGACCTACACCGA
ACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTC
CTGGCCTTTTGCTGGCCTTTTGCTCACATGT
SEQ ID NO: 23-Representative FHL AAV Vector (with ALB Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
GTTCCAGATGGTAAATATACACAAGGGATTTAGTCAAACAATTTTTTGGC
AAGAATATTATGAATTTTGTAATCGGTTGGCAGCCAATGAAATACAAAGA
TGAGTCTAGTTAATAATCTACAATTATTGGTTAAAGACCGGTCTCGAAGG
CCTGCAGGCGGCCGCCGCCACCATGAATGAGACTTCTAGCAAAGATTATT
TGCCTTATGTTATGGGCTATTTGTGTAGCAGAAGATTGCAATGAACTTCC
TCCAAGAAGAAATACAGAAATTCTGACAGGTTCCTGGTCTGACCAAACAT
ATCCAGAAGGCACCCAGGCTATCTATAAATGCCGCCCTGGATATAGATCT
CTTGGAAATGTAATAATGGTATGCAGGAAGGGAGAATGGGTTGCTCTTAA
TCCATTAAGGAAATGTCAGAAAAGGCCCTGTGGACATCCTGGAGATACTC
CTTTTGGTACTTTTACCCTTACAGGAGGAAATGTGTTTGAATATGGTGTA
AAAGCTGTGTATACATGTAATGAGGGGTATCAATTGCTAGGTGAGATTAA
TTACCGTGAATGTGACACAGATGGATGGACCAATGATATTCCTATATGTG
AAGTTGTGAAGTGTTTACCAGTGACAGCACCAGAGAATGGAAAAATTGTC
AGTAGTGCAATGGAACCAGATCGGGAATACCATTTTGGACAAGCAGTACG
GTTTGTATGTAACTCAGGCTACAAGATTGAAGGAGATGAAGAAATGCATT
GTTCAGACGATGGTTTTTGGAGTAAAGAGAAACCAAAGTGTGTGGAAATT
TCATGCAAATCCCCAGATGTTATAAATGGATCTCCTATATCTCAGAAGAT
TATTTATAAGGAGAATGAACGATTTCAATATAAATGTAACATGGGTTATG
AATACAGTGAAAGAGGAGATGCTGTATGCACTGAATCTGGATGGCGTCCG
TTGCCTTCATGTGAAGAAAAATCATGTGATAATCCTTATATTCCAAATGG
TGACTACTCACCTTTAAGGATTAAACACAGAACTGGAGATGAAATCACGT
ACCAGTGTAGAAATGGTTTTTATCCTGCAACCCGGGGAAATACAGCAAAA
TGCACAAGTACTGGCTGGATACCTGCTCCGAGATGTACCTTGAAACCTTG
TGATTATCCAGACATTAAACATGGAGGTCTATATCATGAGAATATGCGTA
GACCATACTTTCCAGTAGCTGTAGGAAAATATTACTCCTATTACTGTGAT
GAACATTTTGAGACTCCGTCAGGAAGTTACTGGGATCACATTCATTGCAC
ACAAGATGGATGGTCGCCAGCAGTACCATGCCTCAGAAAATGTTATTTTC
CTTATTTGGAAAATGGATATAATCAAAATCATGGAAGAAAGTTTGTACAG
GGTAAATCTATAGACGTTGCCTGCCATCCTGGCTACGCTCTTCCAAAAGC
GCAGACCACAGTTACATGTATGGAGAATGGCTGGTCTCCTACTCCCAGAT
GCATCCGTGTCAGCTTTACCCTCTGATAAGATATCGATACATTGATGAGT
TTGGACAAACCACAACTAGAATGCAGTGAAAAAAATGCTTTATTTGTGAA
ATTTGTGATGCTATTGCTTTATTTGTAACCATTATAAGCTGCAATAAACA
AGATATCGTTAACTCGAGGGATCCCACGTGCTGATTTTGTAGGTAACCAC
GTGCGGACCGAGCGGCCGCAGGAACCCCTAGTGATGGAGTTGGCCACTCC
CTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGC
TGCCTGCAGGGGCGCCTGATGCGGTATTTTCTCCTTACGCATCTGTGCGG
TATTTCACACCGCATACGTCAAAGCAACCATAGTACGCGCCCTGTAGCGG
CGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATT
TGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGGCTATTCTTTTGATTTATAAGGGA
TTTTGCCGATTTCGGCCTATTGGTTAAAAAATGAGCTGATTTAACAAAAA
TTTAACGCGAATTTTAACAAAATATTAACGTTTACAATTTTATGGTGCAC
TCTCAGTACAATCTGCTCTGATGCCGCATAGTTAAGCCAGCCCCGACACC
CGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTT
TTCACCGTCATCACCGAAACGCGCGAGACGAAAGGGCCTCGTGATACGCC
TATTTTTATAGGTTAATGTCATGATAATAATGGTTTCTTAGACGTCAGGT
GGCACTTTTCGGGGAAATGTGCGCGGAACCCCTATTTGTTTATTTTTCTA
AATACATTCAAATATGTATCCGCTCATGAGACAATAACCCTGATAAATGC
TTCAATAATATTGAAAAAGGAAGAGTATGAGTATTCAACATTTCCGTGTC
GCCCTTATTCCCTTTTTTGCGGCATTTTGCCTTCCTGTTTTTGCTCACCC
AGAAACGCTGGTGAAAGTAAAAGATGCTGAAGATCAGTTGGGTGCACGAG
TGGGTTACATCGAACTGGATCTCAACAGCGGTAAGATCCTTGAGAGTTTT
CGCCCCGAAGAACGTTTTCCAATGATGAGCACTTTTAAAGTTCTGCTATG
TGGCGCGGTATTATCCCGTATTGACGCCGGGCAAGAGCAACTCGGTCGCC
GCATACACTATTCTCAGAATGACTTGGTTGAGTACTCACCAGTCACAGAA
AAGCATCTTACGGATGGCATGACAGTAAGAGAATTATGCAGTGCTGCCAT
AACCATGAGTGATAACACTGCGGCCAACTTACTTCTGACAACGATCGGAG
GACCGAAGGAGCTAACCGCTTTTTTGCACAACATGGGGGATCATGTAACT
CGCCTTGATCGTTGGGAACCGGAGCTGAATGAAGCCATACCAAACGACGA
GCGTGACACCACGATGCCTGTAGCAATGGCAACAACGTTGCGCAAACTAT
TAACTGGCGAACTACTTACTCTAGCTTCCCGGCAACAATTAATAGACTGG
ATGGAGGCGGATAAAGTTGCAGGACCACTTCTGCGCTCGGCCCTTCCGGC
TGGCTGGTTTATTGCTGATAAATCTGGAGCCGGTGAGCGTGGGTCTCGCG
GTATCATTGCAGCACTGGGGCCAGATGGTAAGCCCTCCCGTATCGTAGTT
ATCTACACGACGGGGAGTCAGGCAACTATGGATGAACGAAATAGACAGAT
CGCTGAGATAGGTGCCTCACTGATTAAGCATTGGTAACTGTCAGACCAAG
TTTACTCATATATACTTTAGATTGATTTAAAACTTCATTTTTAATTTAAA
AGGATCTAGGTGAAGATCCTTTTTGATAATCTCATGACCAAAATCCCTTA
ACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACC
AACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTA
GCACCGCCTACATACCTCGCTCTGCTAATCCTGTTACCAGTGGCTGCTGC
CAGTGGCGATAAGTCGTGTCTTACCGGGTTGGACTCAAGACGATAGTTAC
CGGATAAGGCGCAGCGGTCGGGCTGAACGGGGGGTTCGTGCACACAGCCC
AGCTTGGAGCGAACGACCTACACCGAACTGAGATACCTACAGCGTGAGCT
ATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCT
CACATGT
SEQ ID NO: 24-Representative FHL AAV Vector (with AAT1 Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
GTTCCAGATGGTAAATATACACAAGGGATTTAGTCAAACAATTTTTTGGC
AAGAATATTATGAATTTTGTAATCGGTTGGCAGCCAATGAAATACAAAGA
TGAGTCTAGTTAATAATCTACAATTATTGGTTAAAGACCGGTCTCGAAGG
CCTGCAGGCGGCCGCCGCCACCATGAATGAGACTTCTAGCAAAGATTATT
TGCCTTATGTTATGGGCTATTTGTGTAGCAGAAGATTGCAATGAACTTCC
TCCAAGAAGAAATACAGAAATTCTGACAGGTTCCTGGTCTGACCAAACAT
ATCCAGAAGGCACCCAGGCTATCTATAAATGCCGCCCTGGATATAGATCT
CTTGGAAATGTAATAATGGTATGCAGGAAGGGAGAATGGGTTGCTCTTAA
TCCATTAAGGAAATGTCAGAAAAGGCCCTGTGGACATCCTGGAGATACTC
CTTTTGGTACTTTTACCCTTACAGGAGGAAATGTGTTTGAATATGGTGTA
AAAGCTGTGTATACATGTAATGAGGGGTATCAATTGCTAGGTGAGATTAA
TTACCGTGAATGTGACACAGATGGATGGACCAATGATATTCCTATATGTG
AAGTTGTGAAGTGTTTACCAGTGACAGCACCAGAGAATGGAAAAATTGTC
AGTAGTGCAATGGAACCAGATCGGGAATACCATTTTGGACAAGCAGTACG
GTTTGTATGTAACTCAGGCTACAAGATTGAAGGAGATGAAGAAATGCATT
GTTCAGACGATGGTTTTTGGAGTAAAGAGAAACCAAAGTGTGTGGAAATT
TCATGCAAATCCCCAGATGTTATAAATGGATCTCCTATATCTCAGAAGAT
TATTTATAAGGAGAATGAACGATTTCAATATAAATGTAACATGGGTTATG
AATACAGTGAAAGAGGAGATGCTGTATGCACTGAATCTGGATGGCGTCCG
TTGCCTTCATGTGAAGAAAAATCATGTGATAATCCTTATATTCCAAATGG
TGACTACTCACCTTTAAGGATTAAACACAGAACTGGAGATGAAATCACGT
ACCAGTGTAGAAATGGTTTTTATCCTGCAACCCGGGGAAATACAGCAAAA
TGCACAAGTACTGGCTGGATACCTGCTCCGAGATGTACCTTGAAACCTTG
TGATTATCCAGACATTAAACATGGAGGTCTATATCATGAGAATATGCGTA
GACCATACTTTCCAGTAGCTGTAGGAAAATATTACTCCTATTACTGTGAT
GAACATTTTGAGACTCCGTCAGGAAGTTACTGGGATCACATTCATTGCAC
ACAAGATGGATGGTCGCCAGCAGTACCATGCCTCAGAAAATGTTATTTTC
CTTATTTGGAAAATGGATATAATCAAAATCATGGAAGAAAGTTTGTACAG
GGTAAATCTATAGACGTTGCCTGCCATCCTGGCTACGCTCTTCCAAAAGC
GCAGACCACAGTTACATGTATGGAGAATGGCTGGTCTCCTACTCCCAGAT
GCATCCGTGTCAGCTTTACCCTCTGATAAGATATCGATACATTGATGAGT
TTGGACAAACCACAACTAGAATGCAGTGAAAAAAATGCTTTATTTGTGAA
ATTTGTGATGCTATTGCTTTATTTGTAACCATTATAAGCTGCAATAAACA
AGATATCGTTAACTCGAGGGATCCCACGTGCTGATTTTGTAGGTAACCAC
GTGCGGACCGAGCGGCCGCAGGAACCCCTAGTGATGGAGTTGGCCACTCC
CTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGC
TGCCTGCAGGGGCGCCTGATGCGGTATTTTCTCCTTACGCATCTGTGCGG
TATTTCACACCGCATACGTCAAAGCAACCATAGTACGCGCCCTGTAGCGG
CGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATT
TGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGGCTATTCTTTTGATTTATAAGGGA
TTTTGCCGATTTCGGCCTATTGGTTAAAAAATGAGCTGATTTAACAAAAA
TTTAACGCGAATTTTAACAAAATATTAACGTTTACAATTTTATGGTGCAC
TCTCAGTACAATCTGCTCTGATGCCGCATAGTTAAGCCAGCCCCGACACC
CGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTT
TTCACCGTCATCACCGAAACGCGCGAGACGAAAGGGCCTCGTGATACGCC
TATTTTTATAGGTTAATGTCATGATAATAATGGTTTCTTAGACGTCAGGT
GGCACTTTTCGGGGAAATGTGCGCGGAACCCCTATTTGTTTATTTTTCTA
AATACATTCAAATATGTATCCGCTCATGAGACAATAACCCTGATAAATGC
TTCAATAATATTGAAAAAGGAAGAGTATGAGTATTCAACATTTCCGTGTC
GCCCTTATTCCCTTTTTTGCGGCATTTTGCCTTCCTGTTTTTGCTCACCC
AGAAACGCTGGTGAAAGTAAAAGATGCTGAAGATCAGTTGGGTGCACGAG
TGGGTTACATCGAACTGGATCTCAACAGCGGTAAGATCCTTGAGAGTTTT
CGCCCCGAAGAACGTTTTCCAATGATGAGCACTTTTAAAGTTCTGCTATG
TGGCGCGGTATTATCCCGTATTGACGCCGGGCAAGAGCAACTCGGTCGCC
GCATACACTATTCTCAGAATGACTTGGTTGAGTACTCACCAGTCACAGAA
AAGCATCTTACGGATGGCATGACAGTAAGAGAATTATGCAGTGCTGCCAT
AACCATGAGTGATAACACTGCGGCCAACTTACTTCTGACAACGATCGGAG
GACCGAAGGAGCTAACCGCTTTTTTGCACAACATGGGGGATCATGTAACT
CGCCTTGATCGTTGGGAACCGGAGCTGAATGAAGCCATACCAAACGACGA
GCGTGACACCACGATGCCTGTAGCAATGGCAACAACGTTGCGCAAACTAT
TAACTGGCGAACTACTTACTCTAGCTTCCCGGCAACAATTAATAGACTGG
ATGGAGGCGGATAAAGTTGCAGGACCACTTCTGCGCTCGGCCCTTCCGGC
TGGCTGGTTTATTGCTGATAAATCTGGAGCCGGTGAGCGTGGGTCTCGCG
GTATCATTGCAGCACTGGGGCCAGATGGTAAGCCCTCCCGTATCGTAGTT
ATCTACACGACGGGGAGTCAGGCAACTATGGATGAACGAAATAGACAGAT
CGCTGAGATAGGTGCCTCACTGATTAAGCATTGGTAACTGTCAGACCAAG
TTTACTCATATATACTTTAGATTGATTTAAAACTTCATTTTTAATTTAAA
AGGATCTAGGTGAAGATCCTTTTTGATAATCTCATGACCAAAATCCCTTA
ACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACC
AACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTA
GCACCGCCTACATACCTCGCTCTGCTAATCCTGTTACCAGTGGCTGCTGC
CAGTGGCGATAAGTCGTGTCTTACCGGGTTGGACTCAAGACGATAGTTAC
CGGATAAGGCGCAGCGGTCGGGCTGAACGGGGGGTTCGTGCACACAGCCC
AGCTTGGAGCGAACGACCTACACCGAACTGAGATACCTACAGCGTGAGCT
ATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCT
CACATGT
SEQ ID NO: 25-Representative FHL AAV Vector (with EF1a.SV40i
Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGAC
GCGTTAACTAGTGGGCAGAGCGCACATCGCCCACAGTCCCCGAGAAGTTG
GGGGGAGGGGTCGGCAATTGAACCGGTGCCTAGAGAAGGTGGCGCGGGGT
AAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGC
AACGGGTTTGCCGCCAGAACACAGGTAAGTTTAGTCTTTTTGTCTTTTAT
TTCAGGTCCCGGATCCGGTGGTGGTGCAAATCAAAGAACTGCTCCTCAGT
GGATGTTGCCTTTACTTCTAGGCCTGTACGGAAGTGTTACTTCTGCTCTA
AAAGCTGCGGAATTGTACCCGCGGACCGGTCTCGAAGGCCTGCAGGCGGC
CGCCGCCACCATGAATGAGACTTCTAGCAAAGATTATTTGCCTTATGTTA
TGGGCTATTTGTGTAGCAGAAGATTGCAATGAACTTCCTCCAAGAAGAAA
TACAGAAATTCTGACAGGTTCCTGGTCTGACCAAACATATCCAGAAGGCA
CCCAGGCTATCTATAAATGCCGCCCTGGATATAGATCTCTTGGAAATGTA
ATAATGGTATGCAGGAAGGGAGAATGGGTTGCTCTTAATCCATTAAGGAA
ATGTCAGAAAAGGCCCTGTGGACATCCTGGAGATACTCCTTTTGGTACTT
TTACCCTTACAGGAGGAAATGTGTTTGAATATGGTGTAAAAGCTGTGTAT
ACATGTAATGAGGGGTATCAATTGCTAGGTGAGATTAATTACCGTGAATG
TGACACAGATGGATGGACCAATGATATTCCTATATGTGAAGTTGTGAAGT
GTTTACCAGTGACAGCACCAGAGAATGGAAAAATTGTCAGTAGTGCAATG
GAACCAGATCGGGAATACCATTTTGGACAAGCAGTACGGTTTGTATGTAA
CTCAGGCTACAAGATTGAAGGAGATGAAGAAATGCATTGTTCAGACGATG
GTTTTTGGAGTAAAGAGAAACCAAAGTGTGTGGAAATTTCATGCAAATCC
CCAGATGTTATAAATGGATCTCCTATATCTCAGAAGATTATTTATAAGGA
GAATGAACGATTTCAATATAAATGTAACATGGGTTATGAATACAGTGAAA
GAGGAGATGCTGTATGCACTGAATCTGGATGGCGTCCGTTGCCTTCATGT
GAAGAAAAATCATGTGATAATCCTTATATTCCAAATGGTGACTACTCACC
TTTAAGGATTAAACACAGAACTGGAGATGAAATCACGTACCAGTGTAGAA
ATGGTTTTTATCCTGCAACCCGGGGAAATACAGCAAAATGCACAAGTACT
GGCTGGATACCTGCTCCGAGATGTACCTTGAAACCTTGTGATTATCCAGA
CATTAAACATGGAGGTCTATATCATGAGAATATGCGTAGACCATACTTTC
CAGTAGCTGTAGGAAAATATTACTCCTATTACTGTGATGAACATTTTGAG
ACTCCGTCAGGAAGTTACTGGGATCACATTCATTGCACACAAGATGGATG
GTCGCCAGCAGTACCATGCCTCAGAAAATGTTATTTTCCTTATTTGGAAA
ATGGATATAATCAAAATCATGGAAGAAAGTTTGTACAGGGTAAATCTATA
GACGTTGCCTGCCATCCTGGCTACGCTCTTCCAAAAGCGCAGACCACAGT
TACATGTATGGAGAATGGCTGGTCTCCTACTCCCAGATGCATCCGTGTCA
GCTTTACCCTCTGATAAGATATCGATACATTGATGAGTTTGGACAAACCA
CAACTAGAATGCAGTGAAAAAAATGCTTTATTTGTGAAATTTGTGATGCT
ATTGCTTTATTTGTAACCATTATAAGCTGCAATAAACAAGATATCGTTAA
CTCGAGGGATCCCACGTGCTGATTTTGTAGGTAACCACGTGCGGACCGAG
CGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGGGG
CGCCTGATGCGGTATTTTCTCCTTACGCATCTGTGCGGTATTTCACACCG
CATACGTCAAAGCAACCATAGTACGCGCCCTGTAGCGGCGCATTAAGCGC
GGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATT
TAGTGCTTTACGGCACCTCGACCCCAAAAAACTTGATTTGGGTGATGGTT
CACGTAGTGGGCCATCGCCCTGATAGACGGTTTTTCGCCCTTTGACGTTG
GAGTCCACGTTCTTTAATAGTGGACTCTTGTTCCAAACTGGAACAACACT
CAACCCTATCTCGGGCTATTCTTTTGATTTATAAGGGATTTTGCCGATTT
CGGCCTATTGGTTAAAAAATGAGCTGATTTAACAAAAATTTAACGCGAAT
TTTAACAAAATATTAACGTTTACAATTTTATGGTGCACTCTCAGTACAAT
CTGCTCTGATGCCGCATAGTTAAGCCAGCCCCGACACCCGCCAACACCCG
CTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATC
ACCGAAACGCGCGAGACGAAAGGGCCTCGTGATACGCCTATTTTTATAGG
TTAATGTCATGATAATAATGGTTTCTTAGACGTCAGGTGGCACTTTTCGG
GGAAATGTGCGCGGAACCCCTATTTGTTTATTTTTCTAAATACATTCAAA
TATGTATCCGCTCATGAGACAATAACCCTGATAAATGCTTCAATAATATT
GAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATT
ATCCCGTATTGACGCCGGGCAAGAGCAACTCGGTCGCCGCATACACTATT
CTCAGAATGACTTGGTTGAGTACTCACCAGTCACAGAAAAGCATCTTACG
GATGGCATGACAGTAAGAGAATTATGCAGTGCTGCCATAACCATGAGTGA
TAACACTGCGGCCAACTTACTTCTGACAACGATCGGAGGACCGAAGGAGC
TAACCGCTTTTTTGCACAACATGGGGGATCATGTAACTCGCCTTGATCGT
TGGGAACCGGAGCTGAATGAAGCCATACCAAACGACGAGCGTGACACCAC
GATGCCTGTAGCAATGGCAACAACGTTGCGCAAACTATTAACTGGCGAAC
TACTTACTCTAGCTTCCCGGCAACAATTAATAGACTGGATGGAGGCGGAT
AAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAG
CACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATA
TACTTTAGATTGATTTAAAACTTCATTTTTAATTTAAAAGGATCTAGGTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGT
SEQ ID NO: 26-Representative FHL AAV Vector (with CAG Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGTT
GGCAAAGAATTCTGCAGTCGACGGTACCGCGGGCCCGGGATCCACCGGTC
GCCACCATGGTGCGCTCCTCCAAGAACGTCATCAAGGAGTTCATGCGCTT
CAAGGTGCGCATGGAGGGCACCGTGAACGGCCACGAGTTCGAGATCGAGG
GCGAGGGCGAGGGCCGCCCCTACGAGGGCCACAACACCGTGAAGCTGAAG
GTGACCAAGGGCGGCCCCCTGCCCTTCGCCTGGGACATCCTGTCCCCCCA
GTTCCAGTACGGCTCCAAGGTGTACGTGAAGCACCCCGCCGACATCCCCG
ACTACAAGAAGCTGTCCTTCCCCGAGGGCTTCAAGTGGGAGCGCGTGATG
AACTTCGAGGACGGCGGCGTGGTGACCGTGACCCAGGACTCCTCCCTGCA
GGACGGCTGCTTCATCTACAAGGTGAAGTTCATCGGCGTGAACTTCCCCT
CCGACGGCCCCGTAATGCAGAAGAAGACCATGGGCTGGGAGGCCTCCACC
GAGCGCCTGTACCCCCGCGACGGCGTGCTGAAGGGCGAGATCCACAAGGC
CCTGAAGCTGAAGGACGGCGGCCACTACCTGGTGGAGTTCAAGTCCATCT
ACATGGCCAAGAAGCCCGTGCAGCTGCCCGGCTACTACTACGTGGACTCC
AAGCTGGACATCACCTCCCACAACGAGGACTACACCATCGTGGAGCAGTA
CGAGCGCACCGAGGGCCGCCACCACCTGTTCCTGTAGCGGCCGCACTCCT
CAGGTGCAGGCTGCCTATCAGAAGGTGGTGGCTGGTGTGGCCAATGCCCT
GGCTCACAAATACCACTGAGATCTTTTTCCCTCTGCCAAAAATTATGGGG
ACATCATGAAGCCCCTTGAGCATCTGACTTCTGGCTAATAAAGGAAATTT
ATTTTCATTGCAATAGTGTGTTGGAATTTTTTGTGTCTCTCACTCGGAAG
GACATATGGGAGGGCAAATCACCGGTCTCGAAGGCCTGCAGGCGGCCGCC
GCCACCATGAATGAGACTTCTAGCAAAGATTATTTGCCTTATGTTATGGG
CTATTTGTGTAGCAGAAGATTGCAATGAACTTCCTCCAAGAAGAAATACA
GAAATTCTGACAGGTTCCTGGTCTGACCAAACATATCCAGAAGGCACCCA
GGCTATCTATAAATGCCGCCCTGGATATAGATCTCTTGGAAATGTAATAA
TGGTATGCAGGAAGGGAGAATGGGTTGCTCTTAATCCATTAAGGAAATGT
CAGAAAAGGCCCTGTGGACATCCTGGAGATACTCCTTTTGGTACTTTTAC
CCTTACAGGAGGAAATGTGTTTGAATATGGTGTAAAAGCTGTGTATACAT
GTAATGAGGGGTATCAATTGCTAGGTGAGATTAATTACCGTGAATGTGAC
ACAGATGGATGGACCAATGATATTCCTATATGTGAAGTTGTGAAGTGTTT
ACCAGTGACAGCACCAGAGAATGGAAAAATTGTCAGTAGTGCAATGGAAC
CAGATCGGGAATACCATTTTGGACAAGCAGTACGGTTTGTATGTAACTCA
GGCTACAAGATTGAAGGAGATGAAGAAATGCATTGTTCAGACGATGGTTT
TTGGAGTAAAGAGAAACCAAAGTGTGTGGAAATTTCATGCAAATCCCCAG
ATGTTATAAATGGATCTCCTATATCTCAGAAGATTATTTATAAGGAGAAT
GAACGATTTCAATATAAATGTAACATGGGTTATGAATACAGTGAAAGAGG
AGATGCTGTATGCACTGAATCTGGATGGCGTCCGTTGCCTTCATGTGAAG
AAAAATCATGTGATAATCCTTATATTCCAAATGGTGACTACTCACCTTTA
AGGATTAAACACAGAACTGGAGATGAAATCACGTACCAGTGTAGAAATGG
TTTTTATCCTGCAACCCGGGGAAATACAGCAAAATGCACAAGTACTGGCT
GGATACCTGCTCCGAGATGTACCTTGAAACCTTGTGATTATCCAGACATT
AAACATGGAGGTCTATATCATGAGAATATGCGTAGACCATACTTTCCAGT
AGCTGTAGGAAAATATTACTCCTATTACTGTGATGAACATTTTGAGACTC
CGTCAGGAAGTTACTGGGATCACATTCATTGCACACAAGATGGATGGTCG
CCAGCAGTACCATGCCTCAGAAAATGTTATTTTCCTTATTTGGAAAATGG
ATATAATCAAAATCATGGAAGAAAGTTTGTACAGGGTAAATCTATAGACG
TTGCCTGCCATCCTGGCTACGCTCTTCCAAAAGCGCAGACCACAGTTACA
TGTATGGAGAATGGCTGGTCTCCTACTCCCAGATGCATCCGTGTCAGCTT
TACCCTCTGATAAGATATCGATACATTGATGAGTTTGGACAAACCACAAC
TAGAATGCAGTGAAAAAAATGCTTTATTTGTGAAATTTGTGATGCTATTG
CTTTATTTGTAACCATTATAAGCTGCAATAAACAAGATATCGTTAACTCG
AGGGATCCCACGTGCTGATTTTGTAGGTAACCACGTGCGGACCGAGCGGC
CGCAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCC
TGATGCGGTATTTTCTCCTTACGCATCTGTGCGGTATTTCACACCGCATA
CGTCAAAGCAACCATAGTACGCGCCCTGTAGCGGCGCATTAAGCGCGGCG
GGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGT
GCTTTACGGCACCTCGACCCCAAAAAACTTGATTTGGGTGATGGTTCACG
TAGTGGGCCATCGCCCTGATAGACGGTTTTTCGCCCTTTGACGTTGGAGT
CCACGTTCTTTAATAGTGGACTCTTGTTCCAAACTGGAACAACACTCAAC
CCTATCTCGGGCTATTCTTTTGATTTATAAGGGATTTTGCCGATTTCGGC
CTATTGGTTAAAAAATGAGCTGATTTAACAAAAATTTAACGCGAATTTTA
ACAAAATATTAACGTTTACAATTTTATGGTGCACTCTCAGTACAATCTGC
TCTGATGCCGCATAGTTAAGCCAGCCCCGACACCCGCCAACACCCGCTGA
CGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCG
AAACGCGCGAGACGAAAGGGCCTCGTGATACGCCTATTTTTATAGGTTAA
TGTCATGATAATAATGGTTTCTTAGACGTCAGGTGGCACTTTTCGGGGAA
ATGTGCGCGGAACCCCTATTTGTTTATTTTTCTAAATACATTCAAATATG
TATCCGCTCATGAGACAATAACCCTGATAAATGCTTCAATAATATTGAAA
AAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACT
GGATCTCAACAGCGGTAAGATCCTTGAGAGTTTTCGCCCCGAAGAACGTT
TTCCAATGATGAGCACTTTTAAAGTTCTGCTATGTGGCGCGGTATTATCC
CGTATTGACGCCGGGCAAGAGCAACTCGGTCGCCGCATACACTATTCTCA
GAATGACTTGGTTGAGTACTCACCAGTCACAGAAAAGCATCTTACGGATG
GCATGACAGTAAGAGAATTATGCAGTGCTGCCATAACCATGAGTGATAAC
ACTGCGGCCAACTTACTTCTGACAACGATCGGAGGACCGAAGGAGCTAAC
CGCTTTTTTGCACAACATGGGGGATCATGTAACTCGCCTTGATCGTTGGG
AACCGGAGCTGAATGAAGCCATACCAAACGACGAGCGTGACACCACGATG
CCTGTAGCAATGGCAACAACGTTGCGCAAACTATTAACTGGCGAACTACT
TACTCTAGCTTCCCGGCAACAATTAATAGACTGGATGGAGGCGGATAAAG
TTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACT
GGGGCCAGATGGTAAGCCCTCCCGTATCGTAGTTATCTACACGACGGGGA
GTCAGGCAACTATGGATGAACGAAATAGACAGATCGCTGAGATAGGTGCC
TCACTGATTAAGCATTGGTAACTGTCAGACCAAGTTTACTCATATATACT
TTAGATTGATTTAAAACTTCATTTTTAATTTAAAAGGATCTAGGTGAAGA
TCCTTTTTGATAATCTCATGACCAAAATCCCTTAACGTGAGTTTTCGTTC
CACTGAGCGTCAGACCCCGTAGAAAAGATCAAAGGATCTTCTTGAGATCC
TTTTTTTCTGCGCGTAATCTGCTGCTTGCAAACAAAAAAACCACCGCTAC
CAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTA
GCCGTAGTTAGGCCACCACTTCAAGAACTCTGTAGCACCGCCTACATACC
TCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCG
GTCGGGCTGAACGGGGGGTTCGTGCACACAGCCCAGCTTGGAGCGAACGA
CCTACACCGAACTGAGATACCTACAGCGTGAGCTATGAGAAAGCGCCACG
CTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTT
TTTACGGTTCCTGGCCTTTTGCTGGCCTTTTGCTCACATGT
SEQ ID NO: 27-Representative FHL AAV Vector (with CRALBP
Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGAC
GCGTTAACTAGTACCCTGGTGGTGGTGGTGGGGGGGGGGGGGTGCTCTCT
CAGCAACCCCACCCCGGGATCTTGAGGAGAAAGAGGGCAGAGAAAAGAGG
GAATGGGACTGGCCCAGATCCCAGCCCCACAGCCGGGCTTCCACATGGCC
GAGCAGGAACTCCAGAGCAGGAGCACACAAAGGAGGGCTTTGATGCGCCT
CCAGCCAGGCCCAGGCCTCTCCCCTCTCCCCTTTCTCTCTGGGTCTTCCT
TTGCCCCACTGAGGGCCTCCTGTGAGCCCGATTTAACGGAAACTGTGGGC
GGTGAGAAGTTCCTTATGACACACTAATCCCAACCTGCTGACCGGACCAC
GCCTCCAGCGGAGGGAACCTCTAGAGCTCCAGGACATTCAGGTACCAGGT
AGCCCCAAGGAGGAGCTGCCGACCACCGGTCTCGAAGGCCTGCAGGCGGC
CGCCGCCACCATGAATGAGACTTCTAGCAAAGATTATTTGCCTTATGTTA
TGGGCTATTTGTGTAGCAGAAGATTGCAATGAACTTCCTCCAAGAAGAAA
TACAGAAATTCTGACAGGTTCCTGGTCTGACCAAACATATCCAGAAGGCA
CCCAGGCTATCTATAAATGCCGCCCTGGATATAGATCTCTTGGAAATGTA
ATAATGGTATGCAGGAAGGGAGAATGGGTTGCTCTTAATCCATTAAGGAA
ATGTCAGAAAAGGCCCTGTGGACATCCTGGAGATACTCCTTTTGGTACTT
TTACCCTTACAGGAGGAAATGTGTTTGAATATGGTGTAAAAGCTGTGTAT
ACATGTAATGAGGGGTATCAATTGCTAGGTGAGATTAATTACCGTGAATG
TGACACAGATGGATGGACCAATGATATTCCTATATGTGAAGTTGTGAAGT
GTTTACCAGTGACAGCACCAGAGAATGGAAAAATTGTCAGTAGTGCAATG
GAACCAGATCGGGAATACCATTTTGGACAAGCAGTACGGTTTGTATGTAA
CTCAGGCTACAAGATTGAAGGAGATGAAGAAATGCATTGTTCAGACGATG
GTTTTTGGAGTAAAGAGAAACCAAAGTGTGTGGAAATTTCATGCAAATCC
CCAGATGTTATAAATGGATCTCCTATATCTCAGAAGATTATTTATAAGGA
GAATGAACGATTTCAATATAAATGTAACATGGGTTATGAATACAGTGAAA
GAGGAGATGCTGTATGCACTGAATCTGGATGGCGTCCGTTGCCTTCATGT
GAAGAAAAATCATGTGATAATCCTTATATTCCAAATGGTGACTACTCACC
TTTAAGGATTAAACACAGAACTGGAGATGAAATCACGTACCAGTGTAGAA
ATGGTTTTTATCCTGCAACCCGGGGAAATACAGCAAAATGCACAAGTACT
GGCTGGATACCTGCTCCGAGATGTACCTTGAAACCTTGTGATTATCCAGA
CATTAAACATGGAGGTCTATATCATGAGAATATGCGTAGACCATACTTTC
CAGTAGCTGTAGGAAAATATTACTCCTATTACTGTGATGAACATTTTGAG
ACTCCGTCAGGAAGTTACTGGGATCACATTCATTGCACACAAGATGGATG
GTCGCCAGCAGTACCATGCCTCAGAAAATGTTATTTTCCTTATTTGGAAA
ATGGATATAATCAAAATCATGGAAGAAAGTTTGTACAGGGTAAATCTATA
GACGTTGCCTGCCATCCTGGCTACGCTCTTCCAAAAGCGCAGACCACAGT
TACATGTATGGAGAATGGCTGGTCTCCTACTCCCAGATGCATCCGTGTCA
GCTTTACCCTCTGATAAGATATCGATACATTGATGAGTTTGGACAAACCA
CAACTAGAATGCAGTGAAAAAAATGCTTTATTTGTGAAATTTGTGATGCT
ATTGCTTTATTTGTAACCATTATAAGCTGCAATAAACAAGATATCGTTAA
CTCGAGGGATCCCACGTGCTGATTTTGTAGGTAACCACGTGCGGACCGAG
CGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGGGG
CGCCTGATGCGGTATTTTCTCCTTACGCATCTGTGCGGTATTTCACACCG
CATACGTCAAAGCAACCATAGTACGCGCCCTGTAGCGGCGCATTAAGCGC
GGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATT
TAGTGCTTTACGGCACCTCGACCCCAAAAAACTTGATTTGGGTGATGGTT
CACGTAGTGGGCCATCGCCCTGATAGACGGTTTTTCGCCCTTTGACGTTG
GAGTCCACGTTCTTTAATAGTGGACTCTTGTTCCAAACTGGAACAACACT
CAACCCTATCTCGGGCTATTCTTTTGATTTATAAGGGATTTTGCCGATTT
CGGCCTATTGGTTAAAAAATGAGCTGATTTAACAAAAATTTAACGCGAAT
TTTAACAAAATATTAACGTTTACAATTTTATGGTGCACTCTCAGTACAAT
CTGCTCTGATGCCGCATAGTTAAGCCAGCCCCGACACCCGCCAACACCCG
CTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATC
ACCGAAACGCGCGAGACGAAAGGGCCTCGTGATACGCCTATTTTTATAGG
TTAATGTCATGATAATAATGGTTTCTTAGACGTCAGGTGGCACTTTTCGG
GGAAATGTGCGCGGAACCCCTATTTGTTTATTTTTCTAAATACATTCAAA
TATGTATCCGCTCATGAGACAATAACCCTGATAAATGCTTCAATAATATT
GAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATT
ATCCCGTATTGACGCCGGGCAAGAGCAACTCGGTCGCCGCATACACTATT
CTCAGAATGACTTGGTTGAGTACTCACCAGTCACAGAAAAGCATCTTACG
GATGGCATGACAGTAAGAGAATTATGCAGTGCTGCCATAACCATGAGTGA
TAACACTGCGGCCAACTTACTTCTGACAACGATCGGAGGACCGAAGGAGC
TAACCGCTTTTTTGCACAACATGGGGGATCATGTAACTCGCCTTGATCGT
TGGGAACCGGAGCTGAATGAAGCCATACCAAACGACGAGCGTGACACCAC
GATGCCTGTAGCAATGGCAACAACGTTGCGCAAACTATTAACTGGCGAAC
TACTTACTCTAGCTTCCCGGCAACAATTAATAGACTGGATGGAGGCGGAT
AAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAG
CACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATA
TACTTTAGATTGATTTAAAACTTCATTTTTAATTTAAAAGGATCTAGGTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGT
SEQ ID NO: 28-Representative FHL AAV Vector (with hRPE65
Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGTA
TATTTATTGAAGTTTAATATTGTGTTTGTGATACAGAAGTATTTGCTTTA
ATTCTAAATAAAAATTTTATGCTTTTATTGCTGGTTTAAGAAGATTTGGA
TTATCCTTGTACTTTGAGGAGAAGTTTCTTATTTGAAATATTTTGGAAAC
AGGTCTTTTAATGTGGAAAGATAGATATTAATCTCCTCTTCTATTACTCT
CCAAGATCCAACAAAAGTGATTATACCCCCCAAAATATGATGGTAGTATC
TTATACTACCATCATTTTATAGGCATAGGGCTCTTAGCTGCAAATAATGG
AACTAACTCTAATAAAGCAGAACGCAAATATTGTAAATATTAGAGAGCTA
ACAATCTCTGGGATGGCTAAAGGATGGAGCTTGGAGGCTACCCAGCCAGT
AACAATATTCCGGGCTCCACTGTTGAATGGAGACACTACAACTGCCTTGG
ATGGGCAGAGATATTATGGATGCTAAGCCCCAGGTGCTACCATTAGGACT
TCTACCACTGTCCCTAACGGGTGGAGCCCATCACATGCCTATGCCCTCAC
TGTAAGGAAATGAAGCTACTGTTGTATATCTTGGGAAGCACTTGGATTAA
TTGTTATACAGTTTTGTTGAAGAAGACCCCTAGGGTAAGTAGCCATAACT
GCACACTAAATTTAAAATTGTTAATGAGTTTCTCAAAAAAAATGTTAAGG
TTGTTAGCTGGTATAGTATATATCTTGCCTGTTTTCCAAGGACTTCTTTG
GGCAGTACCTTGTCTGTGCTGGCAAGCAACTGAGACTTAATGAAAGAGTA
TTGGAGATATGAATGAATTGATGCTGTATACTCTCAGAGTGCCAAACATA
TACCAATGGACAAGAAGGTGAGGCAGAGAGCAGACAGGCATTAGTGACAA
GCAAAGATATGCAGAATTTCATTCTCAGCAAATCAAAAGTCCTCAACCTG
GTTGGAAGAATATTGGCACTGAATGGTATCAATAAGGTTGCTAGAGAGGG
TTAGAGGTGCACAATGTGCTTCCATAACATTTTATACTTCTCCAATCTTA
GCACTAATCAAACATGGTTGAATACTTTGTTTACTATAACTCTTACAGAG
TTATAAGATCTGTGAAGACAGGGACAGGGACAATACCCATCTCTGTCTGG
TTCATAGGTGGTATGTAATAGATATTTTTAAAAATAAGTGAGTTAATGAA
TGAGGGTGAGAATGAAGGCACAGAGGTATTAGGGGGAGGTGGGCCCCAGA
GAATGGTGCCAAGGTCCAGTGGGGTGACTGGGATCAGCTCAGGCCTGACG
CTGGCCACTCCCACCTAGCTCCTTTCTTTCTAATCTGTTCTCATTCTCCT
TGGGAAGGATTGAGGTCTCTGGAAAACAGCCAAACAACTGTTATGGGAAC
AGCAAGCCCAAATAAAGCCAAGCATCAGGGGGATCTGAGAGCTGAAAGCA
ACTTCTGTTCCCCCTCCCTCAGCTGAAGGGGTGGGGAAGGGCTCCCAAAG
CCATAACTCCTTTTAAGGGATTTAGAAGGCATAAAAAGGCCCCTGGCTGA
GAACTTCCTTCTTCATTCTGCAGTACCGGTCTCGAAGGCCTGCAGGCGGC
CGCCGCCACCATGAATGAGACTTCTAGCAAAGATTATTTGCCTTATGTTA
TGGGCTATTTGTGTAGCAGAAGATTGCAATGAACTTCCTCCAAGAAGAAA
TACAGAAATTCTGACAGGTTCCTGGTCTGACCAAACATATCCAGAAGGCA
CCCAGGCTATCTATAAATGCCGCCCTGGATATAGATCTCTTGGAAATGTA
ATAATGGTATGCAGGAAGGGAGAATGGGTTGCTCTTAATCCATTAAGGAA
ATGTCAGAAAAGGCCCTGTGGACATCCTGGAGATACTCCTTTTGGTACTT
TTACCCTTACAGGAGGAAATGTGTTTGAATATGGTGTAAAAGCTGTGTAT
ACATGTAATGAGGGGTATCAATTGCTAGGTGAGATTAATTACCGTGAATG
TGACACAGATGGATGGACCAATGATATTCCTATATGTGAAGTTGTGAAGT
GTTTACCAGTGACAGCACCAGAGAATGGAAAAATTGTCAGTAGTGCAATG
GAACCAGATCGGGAATACCATTTTGGACAAGCAGTACGGTTTGTATGTAA
CTCAGGCTACAAGATTGAAGGAGATGAAGAAATGCATTGTTCAGACGATG
GTTTTTGGAGTAAAGAGAAACCAAAGTGTGTGGAAATTTCATGCAAATCC
CCAGATGTTATAAATGGATCTCCTATATCTCAGAAGATTATTTATAAGGA
GAATGAACGATTTCAATATAAATGTAACATGGGTTATGAATACAGTGAAA
GAGGAGATGCTGTATGCACTGAATCTGGATGGCGTCCGTTGCCTTCATGT
GAAGAAAAATCATGTGATAATCCTTATATTCCAAATGGTGACTACTCACC
TTTAAGGATTAAACACAGAACTGGAGATGAAATCACGTACCAGTGTAGAA
ATGGTTTTTATCCTGCAACCCGGGGAAATACAGCAAAATGCACAAGTACT
GGCTGGATACCTGCTCCGAGATGTACCTTGAAACCTTGTGATTATCCAGA
CATTAAACATGGAGGTCTATATCATGAGAATATGCGTAGACCATACTTTC
CAGTAGCTGTAGGAAAATATTACTCCTATTACTGTGATGAACATTTTGAG
ACTCCGTCAGGAAGTTACTGGGATCACATTCATTGCACACAAGATGGATG
GTCGCCAGCAGTACCATGCCTCAGAAAATGTTATTTTCCTTATTTGGAAA
ATGGATATAATCAAAATCATGGAAGAAAGTTTGTACAGGGTAAATCTATA
GACGTTGCCTGCCATCCTGGCTACGCTCTTCCAAAAGCGCAGACCACAGT
TACATGTATGGAGAATGGCTGGTCTCCTACTCCCAGATGCATCCGTGTCA
GCTTTACCCTCTGATAAGATATCGATACATTGATGAGTTTGGACAAACCA
CAACTAGAATGCAGTGAAAAAAATGCTTTATTTGTGAAATTTGTGATGCT
ATTGCTTTATTTGTAACCATTATAAGCTGCAATAAACAAGATATCGTTAA
CTCGAGGGATCCCACGTGCTGATTTTGTAGGTAACCACGTGCGGACCGAG
CGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGGGG
CGCCTGATGCGGTATTTTCTCCTTACGCATCTGTGCGGTATTTCACACCG
CATACGTCAAAGCAACCATAGTACGCGCCCTGTAGCGGCGCATTAAGCGC
GGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATT
TAGTGCTTTACGGCACCTCGACCCCAAAAAACTTGATTTGGGTGATGGTT
CACGTAGTGGGCCATCGCCCTGATAGACGGTTTTTCGCCCTTTGACGTTG
GAGTCCACGTTCTTTAATAGTGGACTCTTGTTCCAAACTGGAACAACACT
CAACCCTATCTCGGGCTATTCTTTTGATTTATAAGGGATTTTGCCGATTT
CGGCCTATTGGTTAAAAAATGAGCTGATTTAACAAAAATTTAACGCGAAT
TTTAACAAAATATTAACGTTTACAATTTTATGGTGCACTCTCAGTACAAT
CTGCTCTGATGCCGCATAGTTAAGCCAGCCCCGACACCCGCCAACACCCG
CTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATC
ACCGAAACGCGCGAGACGAAAGGGCCTCGTGATACGCCTATTTTTATAGG
TTAATGTCATGATAATAATGGTTTCTTAGACGTCAGGTGGCACTTTTCGG
GGAAATGTGCGCGGAACCCCTATTTGTTTATTTTTCTAAATACATTCAAA
TATGTATCCGCTCATGAGACAATAACCCTGATAAATGCTTCAATAATATT
GAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATT
ATCCCGTATTGACGCCGGGCAAGAGCAACTCGGTCGCCGCATACACTATT
CTCAGAATGACTTGGTTGAGTACTCACCAGTCACAGAAAAGCATCTTACG
GATGGCATGACAGTAAGAGAATTATGCAGTGCTGCCATAACCATGAGTGA
TAACACTGCGGCCAACTTACTTCTGACAACGATCGGAGGACCGAAGGAGC
TAACCGCTTTTTTGCACAACATGGGGGATCATGTAACTCGCCTTGATCGT
TGGGAACCGGAGCTGAATGAAGCCATACCAAACGACGAGCGTGACACCAC
GATGCCTGTAGCAATGGCAACAACGTTGCGCAAACTATTAACTGGCGAAC
TACTTACTCTAGCTTCCCGGCAACAATTAATAGACTGGATGGAGGCGGAT
AAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAG
CACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATA
TACTTTAGATTGATTTAAAACTTCATTTTTAATTTAAAAGGATCTAGGTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGT
SEQ ID NO: 29-Representative FHL AAV Vector (with HSP70
Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGTT
AACTAGTCCTGCAGGGCCGCCCACTCCCCCTTCCTCTCAGGGTCCCTGTC
CCCTCCAGTGAATCCCAGAAGACTCTGGAGAGTTCTGAGCAGGGGGCGGC
ACTCTGGCCTCTGATTGGTCCAAGGAAGGCTGGGGGGCAGGACGGGAGGC
GAAAACCCTGGAATATTCCCGACCTGGCAGCCTCATCGAGCTCGGTGATT
GGCTCAGAAGGGAAAAGGCGGGTCTCCGTGACGACTTATAAAAGCCCAGG
GGCAAGCGGTCCGGATAACGGCTAGCCTGAGGAGCTGCTGCGACAGTCCA
CTACCTTTTTCGAGAGTGACTCCCGTTGTCCCAAGGCTTCCCAGAGCGAA
CCTGTGCGGCTGCAGGCACCGGCGCGTCGAGTTTCCGGCGTCCGGAAGGA
CCGAGCTCTTCTCGCGGATCCAGTGTTCCGTTTCCAGCCCCCAATCTCAG
AGCGGAGCCGACAGAGAGCAGGGAACCACCGGTCTCGAAGGCCTGCAGGC
GGCCGCCGCCACCATGAATGAGACTTCTAGCAAAGATTATTTGCCTTATG
TTATGGGCTATTTGTGTAGCAGAAGATTGCAATGAACTTCCTCCAAGAAG
AAATACAGAAATTCTGACAGGTTCCTGGTCTGACCAAACATATCCAGAAG
GCACCCAGGCTATCTATAAATGCCGCCCTGGATATAGATCTCTTGGAAAT
GTAATAATGGTATGCAGGAAGGGAGAATGGGTTGCTCTTAATCCATTAAG
GAAATGTCAGAAAAGGCCCTGTGGACATCCTGGAGATACTCCTTTTGGTA
CTTTTACCCTTACAGGAGGAAATGTGTTTGAATATGGTGTAAAAGCTGTG
TATACATGTAATGAGGGGTATCAATTGCTAGGTGAGATTAATTACCGTGA
ATGTGACACAGATGGATGGACCAATGATATTCCTATATGTGAAGTTGTGA
AGTGTTTACCAGTGACAGCACCAGAGAATGGAAAAATTGTCAGTAGTGCA
ATGGAACCAGATCGGGAATACCATTTTGGACAAGCAGTACGGTTTGTATG
TAACTCAGGCTACAAGATTGAAGGAGATGAAGAAATGCATTGTTCAGACG
ATGGTTTTTGGAGTAAAGAGAAACCAAAGTGTGTGGAAATTTCATGCAAA
TCCCCAGATGTTATAAATGGATCTCCTATATCTCAGAAGATTATTTATAA
GGAGAATGAACGATTTCAATATAAATGTAACATGGGTTATGAATACAGTG
AAAGAGGAGATGCTGTATGCACTGAATCTGGATGGCGTCCGTTGCCTTCA
TGTGAAGAAAAATCATGTGATAATCCTTATATTCCAAATGGTGACTACTC
ACCTTTAAGGATTAAACACAGAACTGGAGATGAAATCACGTACCAGTGTA
GAAATGGTTTTTATCCTGCAACCCGGGGAAATACAGCAAAATGCACAAGT
ACTGGCTGGATACCTGCTCCGAGATGTACCTTGAAACCTTGTGATTATCC
AGACATTAAACATGGAGGTCTATATCATGAGAATATGCGTAGACCATACT
TTCCAGTAGCTGTAGGAAAATATTACTCCTATTACTGTGATGAACATTTT
GAGACTCCGTCAGGAAGTTACTGGGATCACATTCATTGCACACAAGATGG
ATGGTCGCCAGCAGTACCATGCCTCAGAAAATGTTATTTTCCTTATTTGG
AAAATGGATATAATCAAAATCATGGAAGAAAGTTTGTACAGGGTAAATCT
ATAGACGTTGCCTGCCATCCTGGCTACGCTCTTCCAAAAGCGCAGACCAC
AGTTACATGTATGGAGAATGGCTGGTCTCCTACTCCCAGATGCATCCGTG
TCAGCTTTACCCTCTGATAAGATATCGATACATTGATGAGTTTGGACAAA
CCACAACTAGAATGCAGTGAAAAAAATGCTTTATTTGTGAAATTTGTGAT
GCTATTGCTTTATTTGTAACCATTATAAGCTGCAATAAACAAGATATCGT
TAACTCGAGGGATCCCACGTGCTGATTTTGTAGGTAACCACGTGCGGACC
GAGCGGCCGCAGGAACCCCTAGTGATGGAGTTGGCCACTCCCTCTCTGCG
CGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAG
GGGCGCCTGATGCGGTATTTTCTCCTTACGCATCTGTGCGGTATTTCACA
CCGCATACGTCAAAGCAACCATAGTACGCGCCCTGTAGCGGCGCATTAAG
CGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCG
ATTTAGTGCTTTACGGCACCTCGACCCCAAAAAACTTGATTTGGGTGATG
GTTCACGTAGTGGGCCATCGCCCTGATAGACGGTTTTTCGCCCTTTGACG
TTGGAGTCCACGTTCTTTAATAGTGGACTCTTGTTCCAAACTGGAACAAC
ACTCAACCCTATCTCGGGCTATTCTTTTGATTTATAAGGGATTTTGCCGA
TTTCGGCCTATTGGTTAAAAAATGAGCTGATTTAACAAAAATTTAACGCG
AATTTTAACAAAATATTAACGTTTACAATTTTATGGTGCACTCTCAGTAC
AATCTGCTCTGATGCCGCATAGTTAAGCCAGCCCCGACACCCGCCAACAC
CCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTC
ATCACCGAAACGCGCGAGACGAAAGGGCCTCGTGATACGCCTATTTTTAT
AGGTTAATGTCATGATAATAATGGTTTCTTAGACGTCAGGTGGCACTTTT
CGGGGAAATGTGCGCGGAACCCCTATTTGTTTATTTTTCTAAATACATTC
AAATATGTATCCGCTCATGAGACAATAACCCTGATAAATGCTTCAATAAT
ATTGAAAAAGGAAGAGTATGAGTATTCAACATTTCCGTGTCGCCCTTATT
CCCTTTTTTGCGGCATTTTGCCTTCCTGTTTTTGCTCACCCAGAAACGCT
GGTGAAAGTAAAAGATGCTGAAGATCAGTTGGGTGCACGAGTGGGTTACA
TCGAACTGGATCTCAACAGCGGTAAGATCCTTGAGAGTTTTCGCCCCGAA
GAACGTTTTCCAATGATGAGCACTTTTAAAGTTCTGCTATGTGGCGCGGT
ATTATCCCGTATTGACGCCGGGCAAGAGCAACTCGGTCGCCGCATACACT
ATTCTCAGAATGACTTGGTTGAGTACTCACCAGTCACAGAAAAGCATCTT
ACGGATGGCATGACAGTAAGAGAATTATGCAGTGCTGCCATAACCATGAG
TGATAACACTGCGGCCAACTTACTTCTGACAACGATCGGAGGACCGAAGG
AGCTAACCGCTTTTTTGCACAACATGGGGGATCATGTAACTCGCCTTGAT
CGTTGGGAACCGGAGCTGAATGAAGCCATACCAAACGACGAGCGTGACAC
CACGATGCCTGTAGCAATGGCAACAACGTTGCGCAAACTATTAACTGGCG
AACTACTTACTCTAGCTTCCCGGCAACAATTAATAGACTGGATGGAGGCG
GATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTG
CAGCACTGGGGCCAGATGGTAAGCCCTCCCGTATCGTAGTTATCTACACG
ACGGGGAGTCAGGCAACTATGGATGAACGAAATAGACAGATCGCTGAGAT
AGGTGCCTCACTGATTAAGCATTGGTAACTGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGT
SEQ ID NO: 30-Representative FHL AAV Vector (with PCK1
Promoter)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGAG
CCCCCAGTTAGGTTAGGCATTTCCAATCTTTGCCAATAAGCCACATATTT
GCCCAAGTTAGGGTGCATCCTTCCCATGAACTTTGACTGTGACCTTTGAC
TATGGGGTGACATCTTATAGCTGTGGTGTTTTGCCAACCAGCAGCTCTTG
GTACACAAAATGTGCTGCTAGCAGGTGCCCCGGCCAACCTTGTCCTTGAC
CCACCTGCCTGTTAAGAAAAGGGTGTTGTGTTTTGCAACAGCAGTAAAAT
GGGTCAAGGTTTAGTCAGTTGGAAGTTGTGTCAAAACTCACTATGGTTGG
TTGAGGGCTCGAAGTCTCCCAGCATTCATTAACAACTATCTGTTCAATGA
TTATCTCCCTGGGGCGTGTTGCAGTGAGTTGGCCCAAAGCATAACTGACC
CTGGCCGTGATCCAGAGACCTGCCCCCTGACGTCAGTGGCGAGCCTCCCT
GGGTGCAGCTGAGGGGCAGGGCTATTCTTTTCCACAGTACCGGTCTCGAA
GGCCTGCAGGCGGCCGCCGCCACCATGAATGAGACTTCTAGCAAAGATTA
TTTGCCTTATGTTATGGGCTATTTGTGTAGCAGAAGATTGCAATGAACTT
CCTCCAAGAAGAAATACAGAAATTCTGACAGGTTCCTGGTCTGACCAAAC
ATATCCAGAAGGCACCCAGGCTATCTATAAATGCCGCCCTGGATATAGAT
CTCTTGGAAATGTAATAATGGTATGCAGGAAGGGAGAATGGGTTGCTCTT
AATCCATTAAGGAAATGTCAGAAAAGGCCCTGTGGACATCCTGGAGATAC
TCCTTTTGGTACTTTTACCCTTACAGGAGGAAATGTGTTTGAATATGGTG
TAAAAGCTGTGTATACATGTAATGAGGGGTATCAATTGCTAGGTGAGATT
AATTACCGTGAATGTGACACAGATGGATGGACCAATGATATTCCTATATG
TGAAGTTGTGAAGTGTTTACCAGTGACAGCACCAGAGAATGGAAAAATTG
TCAGTAGTGCAATGGAACCAGATCGGGAATACCATTTTGGACAAGCAGTA
CGGTTTGTATGTAACTCAGGCTACAAGATTGAAGGAGATGAAGAAATGCA
TTGTTCAGACGATGGTTTTTGGAGTAAAGAGAAACCAAAGTGTGTGGAAA
TTTCATGCAAATCCCCAGATGTTATAAATGGATCTCCTATATCTCAGAAG
ATTATTTATAAGGAGAATGAACGATTTCAATATAAATGTAACATGGGTTA
TGAATACAGTGAAAGAGGAGATGCTGTATGCACTGAATCTGGATGGCGTC
CGTTGCCTTCATGTGAAGAAAAATCATGTGATAATCCTTATATTCCAAAT
GGTGACTACTCACCTTTAAGGATTAAACACAGAACTGGAGATGAAATCAC
GTACCAGTGTAGAAATGGTTTTTATCCTGCAACCCGGGGAAATACAGCAA
AATGCACAAGTACTGGCTGGATACCTGCTCCGAGATGTACCTTGAAACCT
TGTGATTATCCAGACATTAAACATGGAGGTCTATATCATGAGAATATGCG
TAGACCATACTTTCCAGTAGCTGTAGGAAAATATTACTCCTATTACTGTG
ATGAACATTTTGAGACTCCGTCAGGAAGTTACTGGGATCACATTCATTGC
ACACAAGATGGATGGTCGCCAGCAGTACCATGCCTCAGAAAATGTTATTT
TCCTTATTTGGAAAATGGATATAATCAAAATCATGGAAGAAAGTTTGTAC
AGGGTAAATCTATAGACGTTGCCTGCCATCCTGGCTACGCTCTTCCAAAA
GCGCAGACCACAGTTACATGTATGGAGAATGGCTGGTCTCCTACTCCCAG
ATGCATCCGTGTCAGCTTTACCCTCTGATAAGATATCGATACATTGATGA
GTTTGGACAAACCACAACTAGAATGCAGTGAAAAAAATGCTTTATTTGTG
AAATTTGTGATGCTATTGCTTTATTTGTAACCATTATAAGCTGCAATAAA
CAAGATATCGTTAACTCGAGGGATCCCACGTGCTGATTTTGTAGGTAACC
ACGTGCGGACCGAGCGGCCGCAGGAACCCCTAGTGATGGAGTTGGCCACT
CCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCA
GCTGCCTGCAGGGGCGCCTGATGCGGTATTTTCTCCTTACGCATCTGTGC
GGTATTTCACACCGCATACGTCAAAGCAACCATAGTACGCGCCCTGTAGC
GGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGA
TTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAA
ACTGGAACAACACTCAACCCTATCTCGGGCTATTCTTTTGATTTATAAGG
GATTTTGCCGATTTCGGCCTATTGGTTAAAAAATGAGCTGATTTAACAAA
AATTTAACGCGAATTTTAACAAAATATTAACGTTTACAATTTTATGGTGC
ACTCTCAGTACAATCTGCTCTGATGCCGCATAGTTAAGCCAGCCCCGACA
CCCGCCAACACCCGCTGACGCGCCCTGACGGGCTTGTCTGCTCCCGGCAT
CCGCTTACAGACAAGCTGTGACCGTCTCCGGGAGCTGCATGTGTCAGAGG
TTTTCACCGTCATCACCGAAACGCGCGAGACGAAAGGGCCTCGTGATACG
CCTATTTTTATAGGTTAATGTCATGATAATAATGGTTTCTTAGACGTCAG
GTGGCACTTTTCGGGGAAATGTGCGCGGAACCCCTATTTGTTTATTTTTC
TAAATACATTCAAATATGTATCCGCTCATGAGACAATAACCCTGATAAAT
GCTTCAATAATATTGAAAAAGGAAGAGTATGAGTATTCAACATTTCCGTG
TCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACG
AGTGGGTTACATCGAACTGGATCTCAACAGCGGTAAGATCCTTGAGAGTT
TTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCG
CCGCATACACTATTCTCAGAATGACTTGGTTGAGTACTCACCAGTCACAG
AAAAGCATCTTACGGATGGCATGACAGTAAGAGAATTATGCAGTGCTGCC
ATAACCATGAGTGATAACACTGCGGCCAACTTACTTCTGACAACGATCGG
AGGACCGAAGGAGCTAACCGCTTTTTTGCACAACATGGGGGATCATGTAA
CTCGCCTTGATCGTTGGGAACCGGAGCTGAATGAAGCCATACCAAACGAC
GAGCGTGACACCACGATGCCTGTAGCAATGGCAACAACGTTGCGCAAACT
ATTAACTGGCGAACTACTTACTCTAGCTTCCCGGCAACAATTAATAGACT
GGATGGAGGCGGATAAAGTTGCAGGACCACTTCTGCGCTCGGCCCTTCCG
GCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAG
TTATCTACACGACGGGGAGTCAGGCAACTATGGATGAACGAAATAGACAG
ATCGCTGAGATAGGTGCCTCACTGATTAAGCATTGGTAACTGTCAGACCA
AGTTTACTCATATATACTTTAGATTGATTTAAAACTTCATTTTTAATTTA
AAAGGATCTAGGTGAAGATCCTTTTTGATAATCTCATGACCAAAATCCCT
TAACGTGAGTTTTCGTTCCACTGAGCGTCAGACCCCGTAGAAAAGATCAA
AGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTG
TAGCACCGCCTACATACCTCGCTCTGCTAATCCTGTTACCAGTGGCTGCT
GCCAGTGGCGATAAGTCGTGTCTTACCGGGTTGGACTCAAGACGATAGTT
ACCGGATAAGGCGCAGCGGTCGGGCTGAACGGGGGGTTCGTGCACACAGC
CCAGCTTGGAGCGAACGACCTACACCGAACTGAGATACCTACAGCGTGAG
CTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTG
CTCACATGT
SEQ ID NO: 31-CAG Promoter
GTTAACTTGGCAAAGAATTCTGCAGTCGACGGTACCGCGGGCCCGGGATCCACC
GGTCGCCACCATGGTGCGCTCCTCCAAGAACGTCATCAAGGAGTTCATGCGCTTC
AAGGTGCGCATGGAGGGCACCGTGAACGGCCACGAGTTCGAGATCGAGGGCGA
GGGCGAGGGCCGCCCCTACGAGGGCCACAACACCGTGAAGCTGAAGGTGACCAA
GGGCGGCCCCCTGCCCTTCGCCTGGGACATCCTGTCCCCCCAGTTCCAGTACGGC
TCCAAGGTGTACGTGAAGCACCCCGCCGACATCCCCGACTACAAGAAGCTGTCCT
TCCCCGAGGGCTTCAAGTGGGAGCGCGTGATGAACTTCGAGGACGGCGGCGTGG
TGACCGTGACCCAGGACTCCTCCCTGCAGGACGGCTGCTTCATCTACAAGGTGAA
GTTCATCGGCGTGAACTTCCCCTCCGACGGCCCCGTAATGCAGAAGAAGACCATG
GGCTGGGAGGCCTCCACCGAGCGCCTGTACCCCCGCGACGGCGTGCTGAAGGGC
GAGATCCACAAGGCCCTGAAGCTGAAGGACGGCGGCCACTACCTGGTGGAGTTC
AAGTCCATCTACATGGCCAAGAAGCCCGTGCAGCTGCCCGGCTACTACTACGTGG
ACTCCAAGCTGGACATCACCTCCCACAACGAGGACTACACCATCGTGGAGCAGT
ACGAGCGCACCGAGGGCCGCCACCACCTGTTCCTGTAGCGGCCGCACTCCTCAG
GTGCAGGCTGCCTATCAGAAGGTGGTGGCTGGTGTGGCCAATGCCCTGGCTCACA
AATACCACTGAGATCTTTTTCCCTCTGCCAAAAATTATGGGGACATCATGAAGCC
CCTTGAGCATCTGAcTTCTGGCTAATAAAGGAAATTTATTTTCATTGCAATAGTGT
GTTGGAATTTTTTGTGTCTCTCACTCGGAAGGACATATGGGAGGGCAAATC
SEQ ID NO: 32-hRPE65 Promoter
GTTAACTATATTTATTGAAGTTTAATATTGTGTTTGTGATACAGAAGTATTTGCTT
TAATTCTAAATAAAAATTTTATGCTTTTATTGCTGGTTTAAGAAGATTTGGATTAT
CCTTGTACTTTGAGGAGAAGTTTCTTATTTGAAATATTTTGGAAACAGGTCTTTTA
ATGTGGAAAGATAGATATTAATCTCCTCTTCTATTACTCTCCAAGATCCAACAAA
AGTGATTATACCCCCCAAAATATGATGGTAGTATCTTATACTACCATCATTTTATA
GGCATAGGGCTCTTAGCTGCAAATAATGGAACTAACTCTAATAAAGCAGAACGC
AAATATTGTAAATATTAGAGAGCTAACAATCTCTGGGATGGCTAAAGGATGGAG
CTTGGAGGCTACCCAGCCAGTAACAATATTCCGGGCTCCACTGTTGAATGGAGAC
ACTACAACTGCCTTGGATGGGCAGAGATATTATGGATGCTAAGCCCCAGGTGCTA
CCATTAGGACTTCTACCACTGTCCCTAACGGGTGGAGCCCATCACATGCCTATGC
CCTCACTGTAAGGAAATGAAGCTACTGTTGTATATCTTGGGAAGCACTTGGATTA
ATTGTTATACAGTTTTGTTGAAGAAGACCCCTAGGGTAAGTAGCCATAACTGCAC
ACTAAATTTAAAATTGTTAATGAGTTTCTCAAAAAAAATGTTAAGGTTGTTAGCT
GGTATAGTATATATCTTGCCTGTTTTCCAAGGACTTCTTTGGGCAGTACCTTGTCT
GTGCTGGCAAGCAACTGAGACTTAATGAAAGAGTATTGGAGATATGAATGAATT
GATGCTGTATACTCTCAGAGTGCCAAACATATACCAATGGACAAGAAGGTGAGG
CAGAGAGCAGACAGGCATTAGTGACAAGCAAAGATATGCAGAATTTCATTCTCA
GCAAATCAAAAGTCCTCAACCTGGTTGGAAGAATATTGGCACTGAATGGTATCA
ATAAGGTTGCTAGAGAGGGTTAGAGGTGCACAATGTGCTTCCATAACATTTTATA
CTTCTCCAATCTTAGCACTAATCAAACATGGTTGAATACTTTGTTTACTATAACTC
TTACAGAGTTATAAGATCTGTGAAGACAGGGACAGGGACAATACCCATCTCTGT
CTGGTTCATAGGTGGTATGTAATAGATATTTTTAAAAATAAGTGAGTTAATGAAT
GAGGGTGAGAATGAAGGCACAGAGGTATTAGGGGGAGGTGGGCCCCAGAGAAT
GGTGCCAAGGTCCAGTGGGGTGACTGGGATCAGCTCAGGCCTGACGCTGGCCAC
TCCCACCTAGCTCCTTTCTTTCTAATCTGTTCTCATTCTCCTTGGGAAGGATTGAG
GTCTCTGGAAAACAGCCAAACAACTGTTATGGGAACAGCAAGCCCAAATAAAGC
CAAGCATCAGGGGGATCTGAGAGCTGAAAGCAACTTCTGTTCCCCCTCCCTCAGC
TGAAGGGGTGGGGAAGGGCTCCCAAAGCCATAACTCCTTTTAAGGGATTTAGAA
GGCATAAAAAGGCCCCTGGCTGAGAACTTCCTTCTTCATTCTGCAGTTGG
SEQ ID NO: 33- CFH Amino Acid Sequence
MRLLAKIICLMLWAICVAEDCNELPPRRNTEILTGSWSDQTYPEGTQAIYKCRPGYRS
LG
NVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQ
AVRFVCNSGYKIEGDEEMHCSDDGFWSKEKPKCVEISCKSPDVINGSPISQKIIYKENE
RFQYKCNMGYEYSERGDAVCTESGWRPLPSCEEKSCDNPYIPNGDYSPLRIKHRTGD
EITYQCRNGFYPATRGNTAKCTSTGWIPAPRCTLKPCDYPDIKHGGLYHENMRRPYF
PVAVGKYYSYYCDEHFETPSGSYWDHIHCTQDGWSPAVPCLRKCYFPYLENGYNQN
YGRKFVQGKSIDVACHPGYALPKAQTTVTCMENGWSPTPRCIRVKTCSKSSIDIENGF
ISESQYTYALKEKAKYQCKLGYVTADGETSGSITCGKDGWSAQPTCIKSCDIPVFMN
ARTKNDFTWFKLNDTLDYECHDGYESNTGSTTGSIVCGYNGWSDLPICYERECELPK
IDVHLVPDRKKDQYKVGEVLKFSCKPGFTIVGPNSVQCYHFGLSPDLPICKEQVQSC
GPPPELLNGNVKEKTKEEYGHSEVVEYYCNPRFLMKGPNKIQCVDGEWTTLPVCIVE
ESTCGDIPELEHGWAQLSSPPYYYGDSVEFNCSESFTMIGHRSITCIHGVWTQLPQCV
AIDKLKKCKSSNLIILEEHLKNKKEFDHNSNIRYRCRGKEGWIHTVCINGRWDPEVNC
SMAQIQLCPPPPQIPNSHNMTTTLNYRDGEKVSVLCQENYLIQEGEEITCKDGRWQSI
PLCVEKIPCSQPPQIEHGTINSSRSSQESYAHGTKLSYTCEGGFRISEENETTCYMGKW
SSPPQCEGLPCKSPPEISHGVVAHMSDSYQYGEEVTYKCFEGFGIDGPAIAKCLGEKW
SHPPSCIKTDCLSLPSFENAIPMGEKKDVYKAGEQVTYTCATYYKMDGASNVTCINS
RWTGRPTCRDTSCVNPPTVQNAYIVSRQMSKYPSGERVRYQCRSPYEMFGDEEVMC
LNGNWTEPPQCKDSTGKCGPPPPIDNGDITSFPLSVYAPASSVEYQCQNLYQLEGNK
RITCRNGQWSEPPKCLHPCVISREIMENYNIALRWTAKQKLYSRTGESVEFVCKRGY
RLSSRSHTLRTTCWDGKLEYPTCAK
SEQ ID NO: 34- FHL1 Amino Acid Sequence
MRLLAKIICLMLWAICVAEDCNELPPRRNTEILTGSWSDQTYPEGTQATYKCRPGYRS
LG
NVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQ
AVRFVCNSGYKIEGDEEMHCSDDGFWSKEKPKCVEISCKSPDVINGSPISQKIIYKENE
RFQYKCNMGYEYSERGDAVCTESGWRPLPSCEEKSCDNPYIPNGDYSPLRIKHRTGD
EITYQCRNGFYPATRGNTAKCTSTGWIPAPRCTLKPCDYPDIKHGGLYHENMRRPYF
PVAVGKYYSYYCDEHFETPSGSYWDHIHCTQDGWSPAVPCLRKCYFPYLENGYNQN
YGRKFVQGKSIDVACHPGYALPKAQTTVTCMENGWSPTPRCIRVSFTL
SEQ ID NO: 35- CFI Amino Acid Sequence
MKLLHVFLLFLCFHLRFCKVTYTSQEDLVEKKCLAKKYTHLSCDKVFCQPWQRCIE
GTCVCKLPYQCPKNGTAVCATNRRSFPTYCQQKSLECLHPGTKFLNNGTCTAEGKFS
VSLKHGNTDSEGIVEVKLVDQDKTMFICKSSWSMREANVACLDLGFQQGADTQRRF
KLSDLSINSTECLHVHCRGLETSLAECTFTKRRTMGYQDFADVVCYTQKADSPMDD
FFQCVNGKYISQMKACDGINDCGDQSDELCCKACQGKGFHCKSGVCIPSQYQCNGE
VDCITGEDEVGCAGFASVTQEETEILTADMDAERRRIKSLLPKLSCGVKNRMHIRRK
RIVGGKRAQLGDLPWQVAIKDASGITCGGIYIGGCWILTAAHCLRASKTHRYQIWTT
VVDWIHPDLKRIVIEYVDRIIFHENYNAGTYQNDIALIEMKKDGNKKDCELPRSIPAC
VPWSPYLFQPNDTCIVSGWGREKDNERVFSLQWGEVKLISNCSKFYGNRFYEKEME
CAGTYDGSIDACKGDSGGPLVCMDANNVTYVWGVVSWGENCGKPEFPGVYTKVA
NYFDWISYHVGRPFISQYNV
SEQ ID NO: 36- MECP Promoter Sequence
GGCCGAAATGGACAGGAAATCTCGCCAATTGACGGCATCGCCGCTGAGACTCCC
CCCTCCCCCGTCCTCCCCGTCCCAGCCCGGCCATCACAGCCAATGACGGGCGGGC
TCGCAGCGGCGCCGAGGGCGGGGCGCGGGCGCGCAGGTGCAGCAGCGCGCGGG
CCGGCCAAGAGGGCGGGGCGCGACGTCGGCCGTGCGGGGTCCCGGCGTCGGCGG
CGCGCGC

1. An adeno-associated viral (AAV) vector encoding a Complement Factor H (CFH) or human Factor H Like 1 (FHL1) protein or biologically active fragment thereof, wherein the vector comprises a nucleotide sequence that is at least 80% identical to the nucleotide sequence of SEQ ID NO: 1-3 or 5, or a fragment thereof.

2. The AAV vector of claim 1, wherein the nucleotide sequence is at least 90% identical to the nucleotide sequence of SEQ ID NO: 1-3 or 5, or codon-optimized variant and/or a fragment thereof.

3. The AAV vector of claim 1, wherein the nucleotide sequence is at least 95% identical to the nucleotide sequence of SEQ ID NO: 1-3 or 5, or codon-optimized variant and/or a fragment thereof.

4. The AAV vector of claim 1, wherein the nucleotide sequence is the sequence of SEQ ID NO: 1-3 or 5, or codon-optimized variant and/or a fragment thereof.

5. The AAV vector of any one of claims 1-4, wherein the vector encodes a CFH protein or biologically active fragment thereof comprising at least four CCP domains.

6. The AAV vector of any one of claims 1-4, wherein the vector encodes a CFH protein or biologically active fragment thereof comprising at least five CCP domains.

7. The AAV vector of any one of claims 1-4, wherein the vector encodes a CFH protein or biologically active fragment thereof comprising at least six CCP domains.

8. The AAV vector of any one of claims 1-4, wherein the vector encodes a CFH protein or biologically active fragment thereof comprising at least seven CCP domains.

9. The AAV vector of any one of claims 1-4, wherein the vector encodes a CFH protein or biologically active fragment thereof comprising at least three CCP domains.

10. The AAV vector of any one of claims 1-4, wherein the vector encodes a CFH protein or biologically active fragment thereof comprising the H402 polymorphism.

11. The AAV vector of any one of claims 1-4, wherein the vector encodes a CFH protein or biologically active fragment thereof comprising the V62 polymorphism.

12. The AAV vector of any one of claims 1-11, wherein the CFH protein or biologically active fragment thereof comprises the amino acid sequence of SEQ ID NO: 4.

13. The AAV vector of claim 12, wherein the amino acid sequence of SEQ ID NO: 4 is the C-terminal sequence of the CFH protein.

14. The AAV vector of any one of claims 1-13, wherein the CFH protein or biologically active fragment thereof is capable of diffusing across the Bruch's membrane.

15. The AAV vector of any one of claims 1-14, wherein the CFH protein or biologically active fragment thereof is capable of binding C3b.

16. The AAV vector of any one of claims 1-15, wherein the CFH protein or biologically active fragment thereof is capable of facilitating the breakdown of C3b.

17. The AAV vector of any one of claims 1-16, wherein the vector comprises a promoter that is less than 1000 nucleotides in length.

18. The AAV vector of any one of claims 1-16, wherein the vector comprises a promoter that is less than 500 nucleotides in length.

19. The AAV vector of any one of claims 1-16, wherein the vector comprises a promoter that is less than 400 nucleotides in length.

20. The AAV vector of any one of claims 1-16, wherein the promoter comprises a promoter having the nucleotide sequence of SEQ ID NO: 6, or a fragment thereof.

21. The AAV vector of any one of claims 1-16, wherein the promoter comprises a promoter having the nucleotide sequence of SEQ ID NO: 8, or a fragment thereof.

22. The AAV vector of any one of claims 1-16, wherein the promoter comprises a promoter having the nucleotide sequence of SEQ ID NO: 12, or a fragment thereof.

23. The AAV vector of any one of claims 1-16, wherein the promoter comprises a promoter having the nucleotide sequence of SEQ ID NO: 14, or a fragment thereof.

24. The AAV vector of any one of claims 1-16, wherein the promoter comprises a promoter having the nucleotide sequence of SEQ ID NO: 16, or a fragment thereof.

25. The AAV vector of any one of claims 1-16, wherein the promoter comprises a promoter having the nucleotide sequence of SEQ ID NO: 18, or a fragment thereof.

26. The AAV vector of any one of claims 1-16, wherein the promoter comprises a promoter having the nucleotide sequence of SEQ ID NO: 20, or a fragment thereof.

27. The AAV vector of any one of claims 1-16, wherein the promoter comprises a promoter having the nucleotide sequence of SEQ ID NO: 31, or a fragment thereof.

28. The AAV vector of any one of claims 1-16, wherein the promoter comprises a promoter having the nucleotide sequence of SEQ ID NO: 32, or a fragment thereof.

29. The AAV vector of any one of claims 1-28, wherein the promoter comprises an additional viral intron.

30. The AAV vector of claim 29, wherein the additional viral intron comprises the nucleotide sequence of SEQ ID NO: 10, or a fragment thereof.

31. The AAV vector of any one of claims 1-30, wherein the vector is an AAV2 vector.

32. The AAV vector of any one of claims 1-31, wherein the vector comprises a CMV promoter.

33. The AAV vector of any one of claims 1-32, wherein the vector comprises a Kozak sequence.

34. The AAV vector of any one of claims 1-30, wherein the vector comprises one or more ITR sequence flanking the vector portion encoding CFH.

35. The AAV vector of any one of claims 1-34, wherein the vector comprises a polyadenylation sequence.

36. The AAV vector of any one of claims 1-34, wherein the vector comprises a selective marker.

37. The AAV vector of claim 36, wherein the selective marker is an antibiotic-resistance gene.

38. The AAV vector of claim 37, wherein the antibiotic-resistance gene is an ampicillin-resistance gene.

39. A composition comprising the AAV vector of any one of claims 1-38 and a pharmaceutically acceptable carrier.

40. A method of treating a subject having a disorder associated with undesired activity of the alternative complement pathway, comprising the step of administering to the subject any of the vectors of any one of claims 1-38 or the composition of claim 39.

41. A method of treating a subject having age-related macular degeneration (AMD), comprising the step of administering to the subject any of the vectors of any one of claims 1-38 or the composition of claim 39.

42. The method of claim 40 or 41, wherein the vector or composition is administered intravitreally.

43. The method of any of claims 40-42, wherein the subject is not administered a protease or a polynucleotide encoding a protease.

44. The method of any of claims 40-43, wherein the subject is not administered a furin protease or a polynucleotide encoding a furin protease.

45. The method of any one of claims 40-43, wherein the subject is a human.

46. The method of claim 45, wherein the human is at least 40 years of age.

47. The method of claim 45, wherein the human is at least 50 years of age.

48. The method of claim 45, wherein the human is at least 65 years of age.

49. The method of any one of claims 40-48, wherein the vector or composition is administered locally.

50. The method of any one of claims 40-48, wherein the vector or composition is administered systemically.

51. The method of any one of claims 40-48, wherein the vector or composition comprises a promoter that is associated with strong expression in the liver.

52. The method of claim 51, wherein the promoter comprises a nucleotide sequence that is at least 90%, 95% or 100% identical to the nucleotide sequence of any one of SEQ ID NOs: 16, 18, or 20.

53. The method of any one of claims 40-48, wherein the vector or composition comprises a promoter that is associated with strong expression in the eye.

54. The method of claim 53, wherein the promoter comprises a nucleotide sequence that is at least 90%, 95%, or 100% identical to the nucleotide sequence of any one of SEQ ID NOs: 6 or 32.

55. The method of any one of claims 40-54, wherein the subject has a loss-of-function mutation in the subject's CFI gene.

56. The method of any one of claims 40-55, wherein the subject has one or more CFI mutations selected from the group consisting of: G119R, L131R, V152M, G162D, R187Y, R187T, T2031, A240G, A258T, G287R, A300T, R317W, R339Q, V412M, and P553S.

57. The method of any one of claims 40-56, wherein the subject has a loss-of-function mutation in the subject's CFH gene.

58. The method of any one of claims 40-57, wherein the subject has one or more CFH mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C.

59. The method of any one of claims 40-58, wherein the subject has atypical hemolytic uremic syndrome (aHUS).

60. The method of any one of claims 40-59, wherein the subject is suffering from a renal disease or complication.

61. The vector of any one of claims 1-38 or the composition of claim 39, wherein the vector or composition is capable of inducing at least 20%, 50%, 100%, 150%, 200%, 250%, 300%, 400%, 500%, 700%, 900%, 1000%, 1100%, 1500%, or 2000% higher expression of CFH or FHL in a target cell (e.g., an RPE or liver cell) as compared to the endogenous expression of CFH or FHL in the target cell.

62. The vector of any one of claims 1-38 or the composition of claim 39, wherein the expression of the vector or composition in a target cell (e.g., an RPE or liver cell) results in at least 20%, 50%, 100%, 150%, 200%, 250%, 300%, 400%, 500%, 700%, 900%, 1000%, 1100%, 1500%, or 2000% higher levels of CFH or FHL activity in the target cell as compared to endogenous levels of CFH or FHL activity in the target cell.

63. The vector or composition of any one of claim 1-38, 61 or 62 or the composition of claim 39, wherein the vector or composition induces CFH expression in a target cell of the eye.

64. The vector or composition of claim 63, wherein the vector or composition induces CFH expression in a target cell of the retina or macula.

66. The vector or composition of claim 63 or 64, wherein the target cell of the retina is selected from the group of layers consisting of: inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE).

67. The vector or composition of claim 64, wherein the target cell is in the choroid plexus.

68. The vector or composition of claim 64, wherein the target cell is in the macula.

69. The vector or composition of any one of claim 1-38 or 61-68 wherein the vector or composition induces CFH expression in a cell of the GCL and/or RPE.

70. The method of any one of claims 40-60, wherein the vector or composition is administered to the retina at a dose in the range of 1×10¹⁰ vg/eye to 1×10¹³ vg/eye.

71. The method of claim 70, wherein the vector or composition is administered to the retina at a dose of about 1.4×10¹² vg/eye.

72. The AAV vector of any one of claim 1-16 or 29-38, wherein the promoter comprises a promoter having the nucleotide sequence of SEQ ID NO: 36, or a fragment thereof.
